(19) United States
(12) Patent Application Publication
(10) Pub. No.: US 2020/0055871 A1
(43) Pub. Date: Feb. 20, 2020

(54) ACTIVATORS OF THE RETINOIC ACID INDUCIBLE GENE "RIG-I" PATHWAY AND METHODS OF USE THEREOF

(71) Applicant: Kineta Immuno-Oncology LLC, Seattle, WA (US)

(72) Inventors: Daniel R. Goldberg, Seattle, WA (US); Peter Probst, Seattle, WA (US); Kristin M. Bedard, Bellevue, WA (US)

(21) Appl. No.: 16/536,626

(22) Filed: Aug. 9, 2019

Related U.S. Application Data
(60) Provisional application No. 62/764,944, filed on Aug. 17, 2018.

(51) Int. Cl. C07D 513/04; A61P 35/00

(52) U.S. Cl. CPC C07D 513/04; A61P 35/00

(57) ABSTRACT

The present invention is directed to compounds of Formula (I), which are activators of the RIG-I pathway.

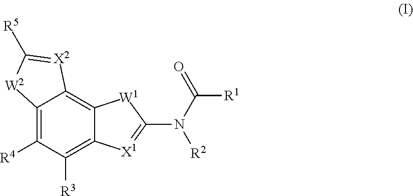

### FIELD OF THE INVENTION

The present invention is directed to compounds and derivatives thereof which are activators of the RIG-I pathway. The present disclosure also relates to the synthesis and to uses of such compounds.

### BACKGROUND OF THE INVENTION

The innate immune system is the first line response against various insults or danger signals including foreign pathogens (e.g., viruses, bacteria and parasites) and cellular damage or abnormalities which may lead to cancer. RIG-I, RIG-I-like receptors (RLRs), Toll-like receptors (TLRs), and the cytosolic DNA receptor, stimulator of interferon genes (STING), are a diverse group of molecules known as pattern-recognition receptors (PRRs). PRRs play a central role in stimulating innate immunity to microbial infections through their ability to recognize pathogen-associated molecular patterns (PAMPs) and signal a cytokine response to control infection. Different PRRs are localized to different cellular compartments, recognize different PAMPs, and signal through different molecular pathways. The common downstream effect is activation of a gene expression program to promote an innate immune response against the invading pathogen. PRRs also play an important role in coordinating the activation and development of the adaptive immune response (Nat Immunol. 2015 April; 16(4):343-353. PMCID: PMC4507498). This includes dendritic cell (DC) recruitment, activation, and antigen presentation to CD8+ T cells. Activation of the transcription factor interferon regulatory factor 3 (IRF3), through RIG-I signaling, is critical for driving DC activation and an antimicrobial response (Immunity. 2014 Nov. 20; 41(5):830-842. PMCID: PMC4384884).

RIG-I recognizes and is activated by viral RNA PAMPs and by endogenous ligands known as damage-associated molecular patterns (DAMPs) that are released during programmed cell death, stress, or tissue injury. Signaling through activated RIG-I, and the resulting transcription factor IRF-3, leads to the induction of an innate immune response that includes the production of cytokines and chemokines; DC recruitment, activation, and antigen uptake; and the presentation of antigens to CD8+ T cells. RIG-I activation is also associated with immunogenic cell death (ICD), a form of programmed cell death in which an immune response is elicited to antigens derived from dying cells (Nat Rev Immunol. 2017 Feb. 17; 17(2):97-111. PMID: 27748397). ICD is also important to overcome immune tolerance mediated by the tumor microenvironment and to elicit an effective immune response against cancer (Oncoimmunology. 2015 April; 4(4):e1008866. PMCID: PMC4485780).

RIG-I is a ubiquitous cytoplasmic protein, and RIG-I RNA is found in all tumor tissues (Vaccine. 2017 Apr. 4; 35(15):1964-1971. PMID: 28279563). Most cancer cells have similar or higher levels of RIG-I protein compared to the level present in normal cells from the same respective tissue and most tumors show moderate to strong cytoplasmic staining for RIG-I by immunohistology (FIG. 2). Interferons and the inflammatory cytokines IL-1β and TNF-α enhance RIG-I expression, whereas the immunosuppressive cytokines IL-10 and TGF-α, abundant in the immune evasive tumor microenvironment, do not control cellular RIG-I levels. Effective immune responses against viruses and tumors share many essential features, and therapeutic benefits of nucleic acid RIG-I ligands (that mimic viral RNA PAMPs) have been demonstrated in several preclinical models of cancer. RIG-I agonists, by inducing ICD and eliciting tumor-targeting T cell populations, may be an effective treatment for cancer, both as a monotherapy or in combination with other cancer immunotherapies. Thus, the use of small-molecule agonists that activate the RIG-I pathway and induce tumor immunity could significantly improve cancer therapies. Accordingly, there is a need for small molecule RIG-I agonists for the treatment of cancer and other diseases. The present invention addresses this and other needs.

### SUMMARY OF THE DISCLOSURE

The present disclosure provides a compound of Formula (I):

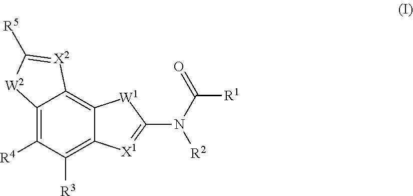

or a pharmaceutically acceptable salt thereof, wherein constituent members are defined herein.

The present disclosure further provides a pharmaceutical composition comprising a compound described herein, or a pharmaceutically acceptable salt thereof, and at least one pharmaceutically acceptable carrier.

The present disclosure further provides a method of activating interferon regulatory factor 3 (IRF3) in a eukaryotic cell, said method comprising contacting a compound described herein, or a pharmaceutically acceptable salt thereof, with IRF3 in said eukaryotic cell.

The present disclosure further provides a method of agonizing retinoic acid-inducible gene-1 pathway (RIG-I) in a eukaryotic cell, said method comprising contacting a compound described herein, or a pharmaceutically acceptable salt thereof, with RIG-I in said eukaryotic cell.

The present disclosure further provides a method of inducing the expression of cytokines that are associated with the RIG-1 pathway in a eukaryotic cell, said method comprising contacting a compound described herein, or a pharmaceutically acceptable salt thereof, with RIG-I in said eukaryotic cell.

The present disclosure further provides a method of inducing immunogenic cell death in a tumor cell of a subject, said method comprising administering to the subject a therapeutically effective amount of a compound described herein, or a pharmaceutically acceptable salt thereof.

The present disclosure further provides a method for treating a cell-proliferation disorder (e.g., cancer) in a subject, said method comprising administering to the subject a therapeutically effective amount of a compound described herein, or a pharmaceutically acceptable salt thereof.

The present disclosure further provides use of a compound described herein, or a pharmaceutically acceptable salt thereof, in therapy.

The present disclosure further provide a compound described herein, or a pharmaceutically acceptable salt thereof, for use in the preparation of a medicament for use in therapy.

The present disclosure further provides a compound described herein, or a pharmaceutically acceptable salt thereof, for use in therapy, such as treating a cell proliferation disorder, for example, cancer.

The present disclosure further provides a compound described herein, or a pharmaceutically acceptable salt thereof, for use in the preparation of a medicament for use in therapy, such as treating a cell proliferation disorder, for example, cancer.

### BRIEF DESCRIPTION OF THE DRAWINGS

FIG. 1 shows compound-induced immunogenic cell death in murine colon carcinoma cells. FIG. 1A shows apoptosis of murine colon carcinoma cells expressed as percentage of Annexin V⁺. FIG. 1B shows calreticulin translocation to cell surface, quantified by mean fluorescent intensity (MFI) of calreticulin⁺ live cells (CRT⁺ LDV⁻).

FIG. 2 shows anti-RIG-I immunohistology results using a representative panel of human cancer tissues (See, The Human Pathology Atlas https://www.proteinatlas.org/humanpathology).

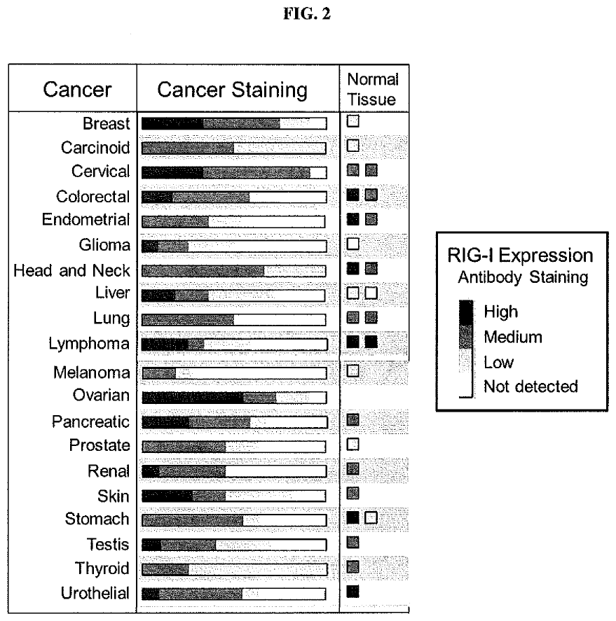

### DETAILED DESCRIPTION OF THE DISCLOSURE

### Compounds

The present invention provides compounds that are activators of the RIG-I pathway. In some embodiments, the present disclosure provides a compound of Formula (I):

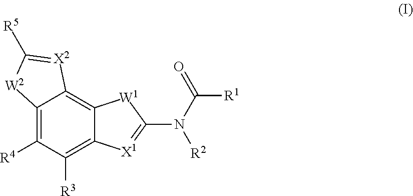

or a pharmaceutically acceptable salt thereof, wherein:

W¹ and W² are each independently selected from O, S, or NH;

X¹ and X² are each independently selected from N or CR^{X};

R^{X} is H or C₁₋₆alkyl;

R¹ is a group having Formula (i), (ii), or (iii):

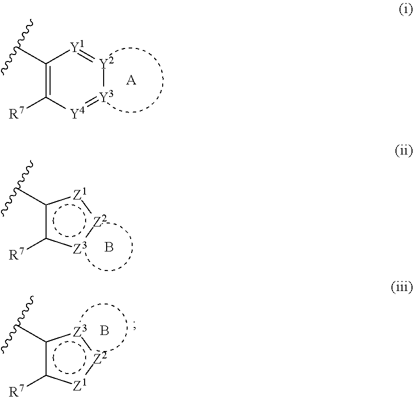

Y¹ is N or CR^{Y1};

Y² is N or CR^{Y2};

Y³ is N or CR^{Y3};

Y⁴ is N or CR^{Y4};

wherein not more than three of Y¹, Y², Y³, and Y⁴ are simultaneously N;

Z¹ is CR^{Z1} or a heteroatom selected from N, O, S or NR^{Z1;}

Z² is CR^{Z2}, or a heteroatom selected from N, O, S or NR^{Z2;};

Z³ is, CR^{Z3} or a heteroatom selected from N, O, S or NR^{Z3;};

wherein the 5-membered ring containing Z¹, Z², and is heteroaromatic and wherein at least one of Z¹, Z² and Z³ is a heteroatom. Ring A is optionally present and represents a fused phenyl group, a fused 5-10 membered heteroaryl group, a fused C₅₋₇cycloalkyl group, or a fused 5-10 membered heterocycloalkyl group, each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein the C₁₋₆ alkyl, C₂₋₆ alkenyl, and C₂₋₆ alkynyl is optionally substituted with 1, 2, or 3 substituents independently selected from Cy¹, Cy¹-C₁₋₄alkyl, halo, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

wherein if Ring A is present, then Y² is CR^{Y2} and Y³ is CR^{Y3} wherein the R^{Y2} and R^{Y3} together with the carbon atoms to which they are attached form Ring A;

Ring B is optionally present and represents a fused phenyl group, a fused 5-10 membered heteroaryl group, a fused C₅₋₇ cycloalkyl group, or a fused 5-10 membered heterocycloalkyl group, each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{e1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein the C₁₋₆ alkyl, C₂₋₆ alkenyl, and C₂₋₆ alkynyl is optionally substituted with 1, 2, or 3 substituents independently selected from Cy¹, Cy¹-C₁₋₄ alkyl, halo, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

wherein if Ring B is present, then Z² is CR^{Z2} and Z³ is CR^{Z3} wherein the R^{Z2} and R^{Z3} together with the carbon atoms to which they are attached form Ring B;

R^{Y1}, R^{Y2}, R^{Y3}, R^{Y4}, R^{Z1}, R^{Z2}, and R^{Z3} are each independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{Y1}, R^{Y2}, R^{Y3}, R^{Y4}, R^{Z1}, R^{Z2}, and R^{Z3} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R² is H or C₁₋₄ alkyl;

R³ is H, halo, C₁₋₄ alkyl, C₁₋₄ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}C(S)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};

R⁴ is H, halo, C₁₋₄ alkyl, C₁₋₄ haloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{c4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}C(S)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{4b}, and S(O)₂NR^{c4}R^{d4};

R⁵ is H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}C(S)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R⁵ are each optionally substituted with 1, 2, 3, 4, or 5 substituents independently selected from Cy⁵, Cy⁵-C₁₋₄ alkyl, halo, C₁₋₆ alkyl, C₂₋₆ alkenyl, C₂₋₆ alkynyl, C₁₋₆ haloalkyl, CN, NO₂, OR^{5a}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{e5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}C(S)NR^{c5}R^{d5}, NR^{c5}S(O)R^{b5}, NR^{e5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};

R⁷ is a group having the formula: —(C₁₋₂ alkyl)ₐ-(L¹)_{b}-(C₂₋₆alkyl)_{c}-(L²)_{d}-Q;

L¹ is —O—, —S—, —NR⁸—, —CO—, —C(O)O—, —CONR⁸—, —SO—, —SO₂—, —SONR⁸—, —S(O)₂NR⁸—, or —NR⁸CONR⁹—;

L² is —O—, —S—, —NR¹⁰—, —CO—, —C(O)O—, —CONR¹⁰—, —SO—, —SO₂—, —SONR¹⁰—, —S(O)₂NR¹⁰—, or —NR¹⁰CONR¹¹—;

R⁸, R⁹, R¹⁰, and R¹¹ are each independently selected from H and C₁₋₄ alkyl;

a is 0 or 1;

b is 0 or 1;

c is 0 or 1;

d is 0 or 1;

wherein the sum of a and c is 1 or 2;

Q is H, halo, C₁₋₆ alkyl, C₂₋₆ alkenyl, C₂₋₆ alkynyl, C₁₋₆ haloalkyl, C₆₋₁₀ aryl, 5-10 membered heteroaryl, C₃₋₁₀ cycloalkyl, 5-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{a}, NR^{c}C(O)NR^{c}R^{d}, NR^{c}(S)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}; wherein the C₁₋₆ alkyl, C₂₋₆ alkenyl, C₂₋₆ alkynyl, C₆₋₁₀ aryl, 5-10 membered heteroaryl, C₃₋₁₀ cycloalkyl, 5-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of Q are each optionally substituted by 1, 2, 3, 4 or 5 substituents selected from halo, C₁₋₆ alkyl, C₂₋₆ alkenyl, C₂₋₆ alkynyl, C₁₋₆ haloalkyl, phenyl, C₃₋₇ cycloalkyl, 5-10 membered heteroaryl, 4-10 membered heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{a}, NR^{c}C(O)NR^{c}R^{d}, NR^{c}C(S)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};

each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀aryl-C ₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(′NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

each Cy⁵ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}C(S)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};

each R^{a}, R^{b}, R^{c}, R^{d}, R^{a1}, R^{b1}, R^{c1}, R^{d1}, R^{a5}, R^{b5}, R^{c5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a5}, R^{b5}, R^{c5}, and R^{d5} is optionally substituted with 1, 2, 3, 4, or 5 substituents independently selected from Cy⁶, Cy⁶-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

each R^{a3}, R^{b3}, R^{c3}, R^{d3}, R^{a4}, R^{b4}, R^{c4}and R^{d4} is independently selected from H and C₁₋₆ alkyl;

or R^{c} and R^{d} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})N^{c6}R^{d6}, S(O)R^{b6}, S(O)NR^{c6}R^{d6}, S(O)₂R^{b6}, NR^{c6}S(O)₂R^{b6}, NR^{e6}S(O)₂NR^{c6}R^{d6}, and S(O)₂NR^{c6}R^{d6};

or R^{c1} and R^{d1} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

or R^{c5} and R^{d5} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{c6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

each Cy⁶ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

each R^{a6}, R^{b6}, R^{c6}, and R^{d6} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

or R^{c6} and R^{d6} together with the N atom to which they are attached form a 3-7 membered heterocycloalkyl group optionally substituted with 1, 2, or 3 substituents independently selected from OH, CN, amino, halo, C₁₋₆ alkyl, C₁₋₆ alkoxy, C₁₋₆haloalkyl, and C₁₋₆ haloalkoxy; and

each R^{e}, R^{e1}, R^{e3}, R^{e4}, R^{e5}, and R^{e6} is independently selected from H, C₁₋₄ alkyl, and CN;

wherein any aforementioned heteroaryl or heterocycloalkyl group comprises 1, 2, 3, or 4 ring-forming heteroatoms independently selected from O, N, and S;

wherein one or more ring-forming C or N atoms of any aforementioned heterocycloalkyl group is optionally substituted by an oxo (═O) group; and

wherein one or more ring-forming S atoms of any aforementioned heterocycloalkyl group is optionally substituted by one or two oxo (═O) groups.

In the moieties of Formula (ii) and (iii),herein, the ring containing Z¹, Z² and Z³ is a heteroaromatic ring. As one skilled in the art understands, for the ring to be heteroaromatic, this ring needs to contain a ring heteroatom, i.e., a ring atom other than carbon. Thus, at least one of Z¹, Z² and Z³is other than a carbon ring atom. Thus, in the formula, with respect to the ring containing Z¹, Z² and Z³. in moieties of formula (ii) or (iii), when ring A or B are absent, then at least one of Z¹, Z² and Z³ is a heteroatom, However, when ring A is present, at least one of Z² and Z³ is N or Z¹ is a heteroatom. Moreover, when ring B is present, at least one of Z² and Z³ is N or Z¹ is a heteroatom.

In an embodiment, when X² is N, W² is S, W¹ is O or S and X¹ is N, then Q is other than H. In another embodiment, when X² is N, W² is S, W¹ is O or S and X¹ is N, then Q may optionally be any one of the following substituents: halo, C₁₋₆ alkyl, C₂₋₆ alkenyl, C₂₋₆ alkynyl, C₁₋₆ haloalkyl, C₆₋₁₀ aryl, which aryl group is unsubstituted or substituted by halo, NR^{c}R^{d}, S(O)₂NR^{c}R^{d}, SR^{a}, alkoxy, aryloxy, arylalkoxy, hydroxy, CN, NO₂, OCF₃, C(O)R^{b}, C(O)NR^{c}R^{d}, C(O)OR^{a}, S(O)R^{b}, S(O)NR^{c}R^{d}, S(O)₂R^{b}, and S(O)₂NR^{c}R^{d}; alkylcarbonyl, 5-10 membered heteroaryl selected from quinoline, isoquinoline, benzodioxanyl, furanyl, thiophene, tetrazolo, thiazole, isothiazole, imidazolo, thiadiazole, thiadiazole S-oxide, thiadiazole-S,S-dioxide, pyrazolo, oxazole, isoxazole, pyridinyl, and pyrimidinyl; heterocycloalkyl selected from morpholinyl, piperidinyl, or dioxanyl, or any combination thereof. In an embodiment, In some embodiments, when X² is N, W² is S, W¹ is O or S and X¹ is N, then Q may optionally not be any one of the following substituents: halo, C₁₋₆ alkyl, C₂₋₆ alkenyl, C₂₋₆ alkynyl, C₁₋₆ haloalkyl, C₆₋₁₀ aryl, which aryl group is unsubstituted or substituted by halo, NR^{c}R^{d}, S(O)₂NR^{c}R^{d}, SR^{a}, alkoxy, aryloxy, arylalkoxy, hydroxy, CN, NO₂, OCF₃, C(O)R^{b}, C(O)NR^{c}R^{d}, C(O)OR^{a}, S(O)R^{b}, S(O)NR^{c}R^{d}, S(O)₂R^{b}, and S(O)₂NR^{c}R^{d}; alkylcarbonyl, 5-10 membered heteroaryl selected from quinoline, isoquinoline, benzodioxanyl,furanyl, thiophene, tetrazolo, thiazole, isothiazole. imidazolo, thiadiazole, thiadiazole S-oxide, thiadiazole-S,S-dioxide, pyrazolo, oxazole, isoxazole, pyridinyl, and pyrimidinyl; heterocycloalkyl selected from morpholinyl, piperidinyl, or dioxanyl, or any combination thereof. In an embodiment, In some embodiments, Q may not be any of the substituents listed in this paragraph when X² is N, W² is S, W¹ is O or S and X¹ is N. In some embodiments, the compound of Formula (I) is other than: N-{3,10-dithia-5,12-diazatricyclo[7.3.0.0²,⁶]dodeca-1,4,6,8,11-pentaen-4-yl}-3-[2-(morpholin-4-yl)ethoxy]naphthalene-2-carboxamide, or a pharmaceutically acceptable salt thereof.

In another embodiment, provided herein is a compound of Formula (I), or a pharmaceutically acceptable salt thereof, wherein:

W¹ and W² are each independently selected from O, S, or NH;

X¹ and X² are each independently selected from N or CR^{X};

R^{X} is H or C₁₋₆ alkyl;

R¹ is a group having Formula (i):

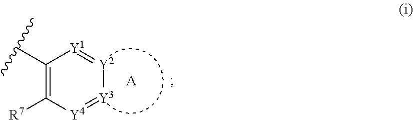

Y¹ is N or CR^{Y1};

Y² is N or CR^{Y2};

Y³ is N or CR^{Y3};

Y⁴ is N or CR^{Y4};

wherein not more than three of Y¹, Y², Y³, and Y⁴ are simultaneously N;

Ring A is a fused phenyl group, a fused 5-10 membered heteroaryl group, a fused C₅₋₇ cycloalkyl group, or a fused 5-10 membered heterocycloalkyl group, each optionally substituted with 1, 2, 3, 4, or 5 substituents independently selected from halo, C₁₋₆ alkyl, C₁₋₆ haloalkyl, CN, NO₂, OR^{a1}, SR^{a1}, C(O)R^{b1}, and NR^{c1}R^{d1};

wherein if Ring A is present, then Y² is CR^{Y2} and Y³ is CR^{Y3} wherein the R^{Y2} and R^{Y3} together with the carbon atoms to which they are attached form Ring A;

R^{Y1}, R^{Y2}, R^{Y3}, and R^{Y4} are each independently selected from H, halo, C₁₋₆ alkyl, C₁₋₆ haloalkyl, C₆₋₁₀ aryl, C₃₋₇ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, S(O)R^{b1}, S(O)NR^{c1}R^{d1}, S(O)₂R^{b1}, and S(O)₂NR^{c1}R^{d1}, wherein said C₁₋₆ alkyl, C₁₋₆ haloalkyl, C₆₋₁₀aryl, C₃₋₇ cycloalkyl, 5-10 membered heteroaryl, and 4-10 membered heterocycloalkyl are each optionally substituted with 1, 2, 3, 4, or 5 substituents independently selected from halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, and OC(O)R^{b1};

R² is H;

R³ is H, halo, C₁₋₄ alkyl, C₁₋₄ haloalkyl, CN, NO₂, OR^{a3}, SR^{a3}, C(O)R^{b3}, C(O)NR^{c3}R^{d3}, C(O)OR^{a3}, NR^{c3}R^{d3}, S(O)NR^{c3}R^{d3}, S(O)₂R^{b3}, or S(O)₂NR^{c3}R^{d3};

R⁴ is H, halo, C₁₋₄ alkyl, C₁₋₄ haloalkyl, CN, NO₂, OR^{a4}, SR^{a4}, C(O)R^{b4}, C(O)NR^{c4}R^{d4}, C(O)OR^{a4}, NR^{c4}R^{d4}, S(O)NR^{c4}R^{d4}, S(O)₂R^{b4}, or S(O)₂NR^{c4}R^{d4};

R⁵ is R⁵ is H, halo, C₁₋₆ alkyl, C₁₋₆ haloalkyl, C₆₋₁₀aryl, C₃₋₇ cycloalkyl, 5-10 membered heteroaryl, 4-10 membered heterocycloalkyl, CN, NO₂, OR^{a5}, SR^{a5}, C(O)R^{b5}, C(O)NR^{c5}R^{d5}, C(O)OR^{a5}, NR^{c5}R^{d5}, S(O)₂R^{b5}, and S(O)₂NR^{c5}R^{d5}; wherein said C₁₋₆ alkyl, C₁₋₆ haloalkyl, C₆₋₁₀ aryl, C₃₋₇ cycloalkyl, 5-10 membered heteroaryl, and 4-10 membered heterocycloalkyl of R⁵ are each optionally substituted with 1, 2, 3, 4, or 5 substituents independently selected from C₁₋₆ alkyl, CN, NO₂, OR^{a5}, SR^{a5}, C(O)R^{b5}, C(O)NR^{c5}R^{d5}, C(O)OR^{a5}, NR^{c5}R^{d5}, S(O)₂R^{b5}, and S(O)₂NR^{c5}R^{d5};

R⁷ is a group having the formula: L¹-(C₂₋₆ alkyl)-Q;

L¹ is —O—, —S—, —NR⁸—, —CO—, —C(O)O—, —CONR⁸—, or —NR⁸CONR⁹—;

Q is H, halo, C₁₋₆ alkyl, C₁₋₆ haloalkyl, C₆₋₁₀ aryl, 5-10 membered heteroaryl, C₃₋₁₀ cycloalkyl, 5-14 membered heterocycloalkyl, CN, NO₂, OR^{a}, SR^{a}, C(O)R^{b}, C(O)NR^{c}R^{d}, C(O)OR^{a}, OC(O)R^{b}, NR^{c}R^{d}, S(O)₂R^{b}, and S(O)₂NR^{c}R^{d}; wherein the C₁₋₆ alkyl, C₁₋₆ haloalkyl, C₆₋₁₀ aryl, 5-10 membered heteroaryl, C₃₋₁₀ cycloalkyl, and 5-14 membered heterocycloalkyl of Q are each optionally substituted by 1, 2, 3, 4 or 5 substituents selected from halo, C₁₋₆ alkyl, C₁₋₆ haloalkyl, phenyl, C₃₋₇ cycloalkyl, 5-10 membered heteroaryl, 4-10 membered heterocycloalkyl, CN, NO₂, OR^{a}, SR^{a}, C(O)R^{b}, NR^{c}R^{d}, S(O)₂R^{b}, and S(O)₂NR^{c}R^{d};

each Cy^{t} is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

each R^{a}, R^{b}, R^{c}, R^{d}, R^{a1}, R^{b1}, R^{c1}, R^{d1}, R^{a5}, R^{b5}, R^{c5}and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a5}, R^{b5}, R^{c5}, and R^{d5} is optionally substituted with 1, 2, 3, 4, or 5 substituents independently selected from Cy⁶, Cy⁶-C₁₋₄ alkyl, halo, C₁₋₄ alkyl, C₁₋₄haloalkyl, C₁₋₆ haloalkyl, C₂₋₆ alkenyl, C₂₋₆ alkynyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

each R^{a3}, R^{b3}, R^{c3}, R^{d3}, R^{a4}, R^{b4}, R^{c4}, and R^{d4} is independently selected from H and C₁₋₆ alkyl;

or R^{c} and R^{d} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{c6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

or R^{c1} and R^{d1} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{c6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

or R^{c5} and R^{d5} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

each Cy⁶ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄alkyl, C₃₋₇ cycloalkyl-C ₁₋₄ alkyl, 5-10 membered heteroaryl-C₁₋₄ alkyl, 4-10 membered heterocycloalkyl-C₁₋₄ alkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};

each R^{a6}, R^{b6}, R^{c6}, and R^{d6} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C ₁₋₆ haloalkoxy;

or R^{c6} and R^{d6} together with the N atom to which they are attached form a 3-7 membered heterocycloalkyl group optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy; and

each R^{e}, R^{e1}, R^{e3}, R^{e4}, R^{e5}, and R^{e6} is independently selected from H, C₁₋₄ alkyl, and CN,

wherein any aforementioned heteroaryl or heterocycloalkyl group comprises 1, 2, 3, or 4 ring-forming heteroatoms independently selected from O, N, and S;

wherein one or more ring-forming C or N atoms of any aforementioned heterocycloalkyl group is optionally substituted by an oxo (═O) group; and

wherein one or more ring-forming S atoms of any aforementioned heterocycloalkyl group is optionally substituted by one or two oxo (═O) groups.

In some embodiments, the compound is other than:

N-{3,10-dithia-5,12-diazatricyclo[7.3.0.0²,⁶]dodeca-1,4,6,8,11-pentaen-4-yl}-3-[2-(morpholin-4-yl)ethoxy]naphthalene-2-carboxamide, or a pharmaceutically acceptable salt thereof.

In some embodiments, W¹ is S.

In some embodiments, W¹ is NH.

In some embodiments, W¹ is O.

In some embodiments, W² is S.

In some embodiments, W² is O.

In some embodiments, W² is NH.

In some embodiments, W¹ and W² are each S.

In some embodiments, X¹ is N.

In some embodiments, X¹ is CH.

In some embodiments, X² is N.

In some embodiments, X² is CH.

In some embodiments, X¹ and X² are each N.

In some embodiments, X¹ and X² are each N and W¹ and W² are each S.

In some embodiments, R¹ is the group having Formula (i):

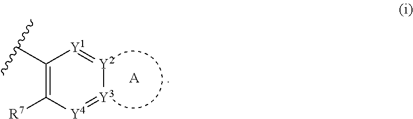

In some embodiments, R¹ is the group having Formula (i-a):

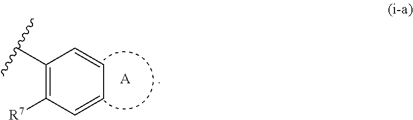

In some embodiments, R¹ is the group having Formula (i-b):

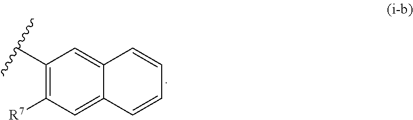

In some embodiments, R¹ is the group having Formula (i-c);

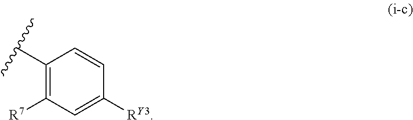

In some embodiments of Formula (i), Y¹ is CR^{Y1}.

In some embodiments of Formula (i), Y² is CR^{Y2}.

In some embodiments of Formula (i), Y³ is CR^{Y3}.

In some embodiments of Formula (i), Y⁴ is CR^{Y4}.

In some embodiments of Formula (i), R^{Y1} is selected from H, halo, C₁₋₆ alkyl, C₁₋₆ haloalkyl, C₆₋₁₀ aryl, C₃₋₇ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, S(O)R^{b1}, S(O)NR^{c1}R^{d1}, S(O)₂R^{b1}, and S(O)₂NR^{c}R^{d1}, wherein said C₁₋₆ alkyl, C₁₋₆ haloalkyl, C₆₋₁₀ aryl, C₃₋₇ cycloalkyl, 5-10 membered heteroaryl, and 4-10 membered heterocycloalkyl are each optionally substituted with 1, 2, 3, 4, or 5 substituents independently selected from halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, and OC(O)R^{b1}.

In some embodiments of Formula (i), R^{Y1}is selected from H, halo, C₁₋₆ alkyl, C₁₋₆ haloalkyl, CN, NO₂, OR^{a1}, SR^{a1}, C(O)R^{b1}, and NR^{c1}R^{d1}.

In some embodiments of Formula (i), R^{Y1} is H.

In some embodiments of Formula (i), R^{Y2} is selected from H, halo, C₁₋₆ alkyl, C₁₋₆ haloalkyl, C₆₋₁₀ aryl, C₃₋₇ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, S(O)R^{b1}, S(O)NR^{c1}R^{d1}, S(O)₂R^{b1}, and S(O)₂NR^{c1}R^{d1}, wherein said C₁₋₆ alkyl, C₁₋₆ haloalkyl, C₆₋₁₀ aryl, C₃₋₇cycloalkyl, 5-10 membered heteroaryl, and 4-10 membered heterocycloalkyl are each optionally substituted with 1, 2, 3, 4, or 5 substituents independently selected from halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, and OC(O)R^{b1}.

In some embodiments of Formula (i), R^{Y2} is selected from H and C₆₋₁₀ aryl, wherein said C₆₋₁₀ aryl is optionally substituted with 1, 2, 3, 4, or 5 substituents independently selected from halo, C₁₋₆ alkyl, C₁₋₆ haloalkyl, CN, NO₂, OR^{a1}, SR^{b1}, C(O)R^{b1}, C(O)NR^{c1}R^{d1}, C(O)OR^{a1}, and OC(O)R^{b1}.

In some embodiments of Formula (i), R^{Y2} is H or C₆₋₁₀ aryl.

In some embodiments of Formula (i), R^{Y2} is H.

In some embodiments of Formula (i), R^{Y3} is selected from H, halo, C₁₋₆ alkyl, C₁₋₆ haloalkyl, C₆₋₁₀ aryl, C₃₋₇ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, S(O)R^{b1}, S(O)NR^{c1}R^{d1}, S(O)₂R^{b1}, and S(O)₂NR^{c1}R^{d1}, wherein said C₁₋₆ alkyl, C₁₋₆ haloalkyl, C₆₋₁₀aryl, C₃₋₇cycloalkyl, 5-10 membered heteroaryl, and 4-10 membered heterocycloalkyl are each optionally substituted with 1, 2, 3, 4, or 5 substituents independently selected from halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, , and OC(O)R^{b1}.

In some embodiments of Formula (i), R^{Y3} is selected from H and C₆₋₁₀ aryl, wherein said C₆₋₁₀ aryl is optionally substituted with 1, 2, 3, 4, or 5 substituents independently selected from halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, and OC(O)R^{b1}.

In some embodiments of Formula (i), R^{Y3} is H or C₆₋₁₀ aryl

In some embodiments of Formula (i), R^{Y3} is H or phenyl.

In some embodiments of Formula (i), R^{Y3} is H.

In some embodiments of Formula (i), R^{Y4} is selected from H, halo, C₁₋₆ alkyl, C₁₋₆ haloalkyl, C₆₋₁₀ aryl, C₃₋₇ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, S(O)R^{b1}, S(O)NR^{c1}R^{d1}, S(O)₂R^{b1}, and S(O)₂NR^{c1}R^{d1}, wherein said C₁₋₆ alkyl, C₁₋₆ haloalkyl, C₆₋₁₀ aryl, C₃₋₇ cycloalkyl, 5-10 membered heteroaryl, and 4-10 membered heterocycloalkyl are each optionally substituted with 1, 2, 3, 4, or 5 substituents independently selected from halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, and OC(O)R^{b1}.

In some embodiments of Formula (i), R^{Y4} is selected from H, halo, C₁₋₆ alkyl, C₁₋₆ haloalkyl, CN, NO₂, OR^{a1}, SR^{a1}, C(O)R^{b1}, and NR^{c1}R^{d1}.

In some embodiments of Formula (i), R^{Y4} is H.

In some embodiments of Formula (i), Y² is CR^{Y2} and Y³ is CR^{Y3}, and wherein the R^{Y2} and R^{Y3} together with the carbon atoms to which they are attached form Ring A.

In some embodiments of Formula (i) or Formula (i-a), Ring A is a fused phenyl group, a fused 5-10 membered heteroaryl group, a fused C₅₋₇ cycloalkyl group, or a fused 5-10 membered heterocycloalkyl group, each optionally substituted with 1, 2, 3, 4, or 5 substituents independently selected from halo, C₁₋₆ alkyl, C₁₋₆ haloalkyl, CN, NO₂, OR^{a1}, SR^{a1}, C(O)R^{b1}, and NR^{c1}R^{d1}.

In some embodiments of Formula (i) or Formula (i-a), Ring A is a fused phenyl group, a fused 5-10 membered heteroaryl group, a fused C₅₋₇ cycloalkyl group, or a fused 5-10 membered heterocycloalkyl group.

In some embodiments of Formula (i) or Formula (i-a), Ring A is a fused phenyl group.

In some embodiments of Formula (i) or Formula (i-a), A is a fused phenyl group, fused 1,3-dioxolanyl group, fused thiophenyl group, or fused pyrrolyl group.

In some embodiments of Formula (i) or Formula (i-a), A is absent.

In some embodiments, R¹ is the group having Formula (ii):

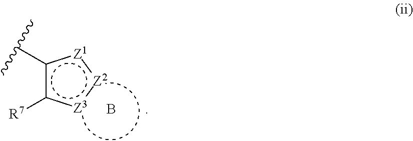

In some embodiments of Formula (ii), Z¹ is O, S, or NR^{Z1}.

In some embodiments of Formula (ii), Z² is N, CR^{Z2}, or NR^{Z2}.

In some embodiments of Formula (ii), Z³ is N, CR^{Z3}, or NR^{Z3}.

In some embodiments of Formula (ii), R^{Z1}, R^{Z2}, and R^{Z3} are each independently selected from H, halo, and C₁₋₆ alkyl.

In some embodiments, R¹ is the group having Formula (iii):

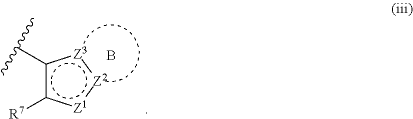

In some embodiments of Formula (iii), Z¹ is N, CR^{Z1}, or NR^{Z1}.

In some embodiments of Formula (iii), Z² is N, CR^{Z2},or NR^{Z2}.

In some embodiments of Formula (iii), Z³ is O, S, or NR^{Z3}.

In some embodiments of Formula (iii), R^{Z1}, R^{Z2}, and R^{Z3} are each independently selected from H, halo, and C₁₋₆ alkyl.

In some embodiments, a is 0.

In some embodiments, b is 1.

In some embodiments, c is 1.

In some embodiments, d is 0.

In some embodiments, R⁷ is a group having the formula: -L¹-(C₂₋₆ alkyl)-Q.

In some embodiments, R⁷ is a group having the formula:

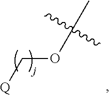

wherein j is 2, 3, 4, 5, or 6.

In some embodiments, R⁷ is a group having the formula:

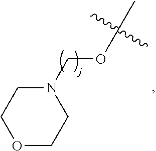

wherein j is 2, 3, 4, 5, or 6. In some embodiments, R¹ is also

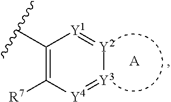

wherein Y¹, Y², Y³ and Y⁴ are all CH and Ring A is either absent or is a fused phenyl ring (that is phenyl or naphthyl). In some embodiments, R⁵ is also H, OR^{a5} or SR^{a5}. For example, in this embodiment, R⁵ is H C₁₋₆ alkyl, C₁₋₆ alkoxy, C₁₋₆-thioalkyl, or hydroxyl.

In some embodiments, L¹ is —O—, —S—, —NR⁸—, —CO—, —C(O)O—, —CONR⁸—, or —NR⁸CONR⁹—.

In some embodiments, L¹ is —O—, —S—, or —NR⁸—.

In some embodiments, L¹ is —O—.

In some embodiments, Q is selected from H, halo, C₁₋₆ alkyl, C₁₋₆ haloalkyl, C₆₋₁₀ aryl, 5-10 membered heteroaryl, C₃₋₁₀ cycloalkyl, 5-14 membered heterocycloalkyl, CN, NO₂, OR^{a}, SR^{a}, C(O)R^{b}, C(O)NR^{c}R^{d}, C(O)OR^{a}, OC(O)R^{b}, NR^{c}R^{d}, S(O)₂R^{b}, and S(O)₂NR^{c}R^{d}; wherein the C₁₋₆ alkyl, C₁₋₆ haloalkyl, C₆₋₁₀ aryl, 5-10 membered heteroaryl, C₃₋₁₀cycloalkyl, and 5-14 membered heterocycloalkyl of Q are each optionally substituted by 1, 2, 3, 4 or 5 substituents selected from halo, C₁₋₆ alkyl, C₁₋₆ haloalkyl, phenyl, C₃₋₇ cycloalkyl, 5-10 membered heteroaryl, 4-10 membered heterocycloalkyl, CN, NO₂, OR^{a}, SR^{a}, C(O)R^{b}, NR^{c}R^{d}, S(O)₂R^{b}, and S(O)₂NR^{c}R^{d}.

In some embodiments, Q is selected from halo, C₁₋₆ alkyl, C₁₋₆ haloalkyl, C₆₋₁₀ aryl, 5-10 membered heteroaryl, C₃₋₁₀ cycloalkyl, 5-14 membered heterocycloalkyl, CN, NO₂, OR^{a}, SR^{a}, C(O)R^{b}, C(O)NR^{c}R^{d}, C(O)OR^{a}, OC(O)R^{b}, NR^{c}R^{d}, S(O)₂R^{b}, and S(O)₂NR^{a}R^{d}; wherein the C₁₋₆ alkyl, C₁₋₆ haloalkyl, C₆₋₁₀ aryl, 5-10 membered heteroaryl, C₃₋₁₀ cycloalkyl, and 5-14 membered heterocycloalkyl of Q are each optionally substituted by 1, 2, 3, 4 or 5 substituents selected from halo, C₁₋₆ alkyl, C₁₋₆ haloalkyl, phenyl, C₃₋₇ cycloalkyl, 5-10 membered heteroaryl, 4-10 membered heterocycloalkyl, CN, NO₂, OR^{a}, SR^{a}, C(O)R^{b}, NR^{c}R^{d}, S(O)₂R^{b}, and S(O)₂NR^{c}R^{d}.

In some embodiments, Q is 5-14 membered heterocycloalkyl or NR^{c}R^{d}, wherein said 5-14 membered heterocycloalkyl is optionally substituted by 1, 2, 3, 4 or 5 substituents selected from halo, C₁₋₆ alkyl, C₁₋₆ haloalkyl, phenyl, C₃₋₇ cycloalkyl, 5-10 membered heteroaryl, 4-10 membered heterocycloalkyl, CN, NO₂, OR^{a}, SR^{a}, C(O)R^{b}, NR^{c}R^{d}, S(O)₂R^{b}, and S(O)₂NR^{c}R^{d}.

In some embodiments, Q is 5-14 membered heterocycloalkyl or NR^{c}R^{d}.

In some embodiments, Q is morpholinyl, piperidinyl, 2-oxa-6-azaspiro[3.3]heptanyl, 2-oxa-5-azabicyclo[2.2.1]heptanyl, or piperazinyl.

In some embodiments, Q is NR^{c}R^{d}.

In some embodiments, R^{c} is H or C₁₋₆ alkyl, wherein said C₁₋₆ alkyl is optionally substituted with OR^{a6}.

In some embodiments, R^{d} is H or C₁₋₆ alkyl, wherein said C₁₋₆ alkyl is optionally substituted with OR^{a6}.

In some embodiments, R² is H.

In some embodiments, R³ is H, halo, C₁₋₄ alkyl, C₁₋₄ haloalkyl, CN, NO₂, OR^{a3}, SR^{a3}, C(O)R^{b3}, C(O)NR^{c3}R^{d3}, C(O)OR^{a3}, NR^{c3}R^{d3}, S(O)NR^{c3}R^{d3}, S(O)₂R^{b3}, or S(O)₂NR^{c3}R^{d3}.

In some embodiments, R³ is H, halo, or C₁₋₄ alkyl.

In some embodiments, R³ is H.

In some embodiments, R⁴ is H, halo, C₁₋₄ alkyl, C₁₋₄ haloalkyl, CN, NO₂, OR^{a4}, SR^{a4}, C(O)R^{b4}, C(O)NR^{c4}R^{d4}, C(O)OR^{a4}, NR^{c4}R^{d4}, S(O)NR^{c4}R^{d4}, S(O)₂R^{b4}, or S(O)₂NR^{c4}R^{d4}.

In some embodiments, R⁴ is H, halo, or C₁₋₄ alkyl.

In some embodiments, R⁴ is H.

In some embodiments, R⁵ is selected from H, halo, C₁₋₆ alkyl, C₁₋₆ haloalkyl, C₆₋₁₀ aryl, C₃₋₇ cycloalkyl, 5-10 membered heteroaryl, 4-10 membered heterocycloalkyl, CN, NO₂, OR^{a5}, SR^{a5}, C(O)R^{b5}, C(O)NR^{c5}R^{d5}, C(O)OR^{a5}, NR^{c5}R^{d5}, S(O)₂R^{b5}, and S(O)2NR^{c5}R^{d5}; wherein said C₁₋₆ alkyl, C₁₋₆ haloalkyl, C₆₋₁₀ aryl, C₃₋₇ cycloalkyl, 5-10 membered heteroaryl, and 4-10 membered heterocycloalkyl of R⁵ are each optionally substituted with 1, 2, 3, 4, or 5 substituents independently selected from C₁₋₆ alkyl, CN, NO₂, OR^{a5}, SR^{a5}, C(O)R^{b5}, C(O)NR^{c5}R^{d5}, C(O)OR^{a5}, NR^{c5}R^{d5}, S(O)₂R^{b5}, and S(O)₂NR^{c5}R^{d5}.

In some embodiments, R⁵ is selected from H, halo, C₁₋₆ alkyl, C₁₋₆ haloalkyl, CN, NO₂, OR^{a5}, SR^{a5}, C(O)R^{b5}, C(O)NR^{c5}R^{d5}, C(O)OR^{a5}, NR^{c5}R^{d5}, S(O)₂R^{b5}, and S(O)₂NR^{c5}R^{d5}.

In some embodiments, R⁵ is OR^{a5} or SR^{a5}.

In some embodiments, R⁵ is H, OCH₃, or SCH₃,

In some embodiments, R⁵ is H.

In some embodiments, provided herein is a compound having Formula IIa:

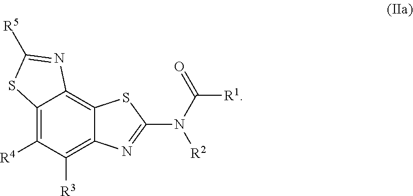

In some embodiments, provided herein is a compound having Formula IIb:

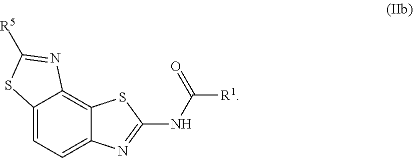

In some embodiments, provided herein is a compound having Formula IIC:

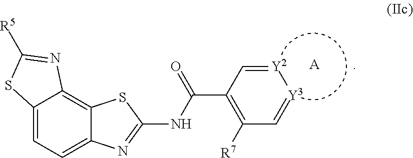

In some embodiments, provided herein is a compound having Formula IId:

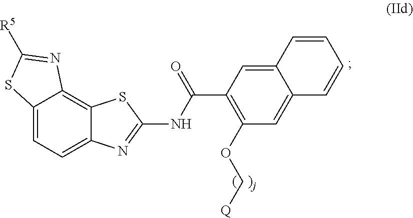

wherein j is 2, 3, 4, 5, or 6. In some embodiments, Q is also morpholinyl. In some of these embodiments, R⁵ is also H, OR^{a5} or SR^{a5}, for example, in this embodiment, R⁵ is H C₁₋₆ alkyl, C₁₋₆ alkoxy, C₁₋₆ thioalkyl, or hydroxyl.

In some embodiments, provided herein is a compound having Formula IIIa:

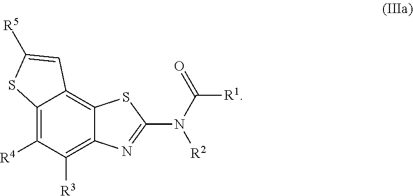

In some embodiments, provided herein is a compound having Formula IVa:

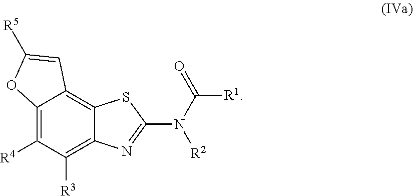

In some embodiments, provided herein is a compound having Formula Va:

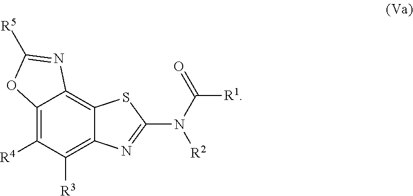

In some embodiments, provided herein is a compound having Formula VIa:

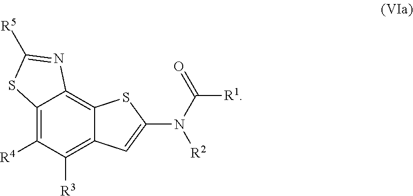

In some embodiments, provided herein is a compound having Formula VIb:

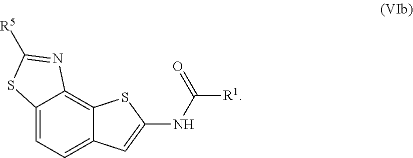

In some embodiments, provided herein is a compound having Formula VIc:

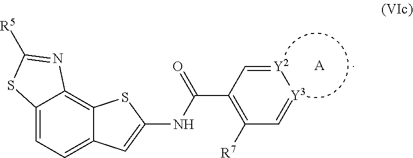

In some embodiments, provided herein is a compound having Formula VId:

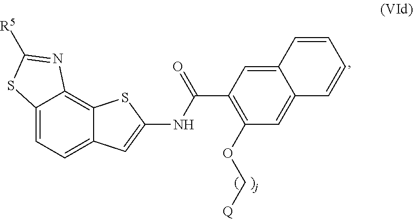

wherein j is 2, 3, 4, 5, or 6. In some embodiments, Q is also morpholinyl. In some of these embodiments, R⁵ is also H, OR^{a5} or SR^{a5}, for example, in this embodiment, R⁵ is H C₁₋₆ alkyl, C₁₋₆ alkoxy, C₁₋₆ thioalkyl, or hydroxyl.

In some embodiments, provided herein is a compound having Formula VIIa:

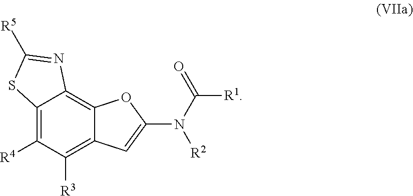

In some embodiments, provided herein is a compound having Formula VIIIa:

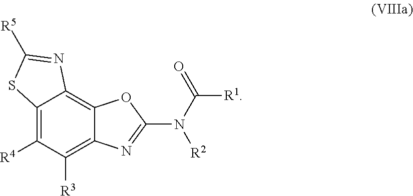

In some embodiments, provided herein is a compound having Formula IXa:

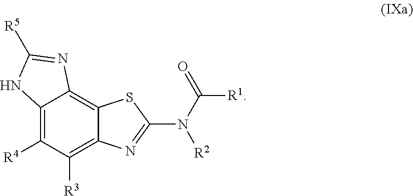

With respect to any formula(e) herein, X¹, X², W1, W², R1, R², R³, R⁴, R⁵, R⁷, R⁸, R⁹, R¹⁰, R¹¹, R^{x}, Y¹, Y², R^{Y3}, Y⁴, R⁷, Ring B, R^{Y1}, R^{Y2}, R^{Y4}, R^{Z1}, R^{Z2}, R^{Z3}, L¹. L², a, b, c, d, Q, Cy¹, Cy⁵, Cy⁶, R^{a}, R^{b}, R^{c}, R^{d}, R^{e}, R^{a1}, R^{b1}, R^{c1}, R^{d1}, R^{e1}. R^{a2}, R^{e2}, R^{a3}, R^{b3}, R^{c3}, R^{d3}, R^{e3}, R^{a4}, R^{b4}, R^{c4}, R^{d4}, R^{e4}, R^{a5}, R^{b5}, R^{c5}, R^{d5}, R^{e5}, R^{a6}, R^{b6}, R^{c6}, R^{d6} and R^{e6} are each as defined herein.

In some embodiments, the compound of Formula (I) is selected from:

N-{3,10-dithia-5,12-diazatricyclo[7.3.0.0²,⁶]dodeca-1,4,6,8,11-pentaen-4-yl}-3-[2-(piperidin-1-yl)ethoxy]naphthalene-2-carboxamide;

N-{3,10-dithia-5,12-diazatricyclo[7.3.0.0²,⁶]dodeca-1,4,6,8,11-pentaen-4-yl}-3-[3-(morpholin-4-yl)propoxy]naphthalene-2-carboxamide;

N-{3,10-dithia-5,12-diazatricyclo[7.3.0.0²,⁶]dodeca-1,4,6,8,11-pentaen-4-yl}-3-[2-(oxan-4-yl)ethoxy]naphthalene-2-carboxamide;

N-{3,10-dithia-5,12-diazatricyclo[7.3.0.0²,]dodeca-1,4,6,8,11-pentaen-4-yl}-3-[4-(morpholin-4-yl)butoxy]naphthalene-2-carboxamide;

N-{11-methoxy-3,10-dithia-5,12-diazatricyclo[7.3.0.0²,⁶]dodeca-1,4,6,8,11-pentaen-4-yl}-3-[2-(morpholin-4-yl)ethoxy]naphthalene-2-carboxamide;

N-{11-methoxy-3,10-dithia-5,12-diazatricyclo[7.3.0.0²,⁶]dodeca-1,4,6,8,11-pentaen-4-yl}-3-[2-(piperidin-1-yl)ethoxy]naphthalene-2-carboxamide;

N-{11-methoxy-3,10-dithia-5,12-diazatricyclo[7.3.0.0²,⁶]dodeca-1,4,6,8,11-pentaen-4-yl}-3-[3-(morpholin-4-yl)propoxy]naphthalene-2-carboxamide;

N-{11-methoxy-3,10-dithia-5,12-diazatricyclo[7.3.0.0²,⁶]dodeca-1,4,6,8,11-pentaen-4-yl}-3-[4-(morpholin-4-yl)butoxy]naphthalene-2-carboxamide;

N-{3,10-dithia-5,12-diazatricyclo[7.3.0.0²^{⁶},]dodeca-1,4,6,8,11-pentaen-4-yl}-3-(2-{2-oxa-5-azabicyclo[2.2.1]heptan-5-yl}ethoxy)naphthalene-2-carboxamide;

N-{3,10-dithia-5,12-diazatricyclo[7.3.0.0²,⁶]dodeca-1,4,6,8,11-pentaen-4-yl}-3-(2-{2-oxa-6-azaspiro[3.3]heptan-6-yl}ethoxy)naphthalene-2-carboxamide;

N-[11-(methylsulfanyl)-3,10-dithia-5,12-diazatricyclo[7.3.0.0²,⁶]dodeca-1,4,6,8,11-pentaen-4-yl]-3-[2-(morpholin-4-yl)ethoxy]naphthalene-2-carboxamide;

N-[11-(methylsulfanyl)-3,10-dithia-5,12-diazatricyclo[7.3.0.0²,⁶]dodeca-1,4,6,8,11-pentaen-4-yl]-3-[3-(morpholin-4-yl)propoxy]naphthalene-2-carboxamide;

N-[11-(methylsulfanyl)-3,10-dithia-5,12-diazatricyclo[7.3.0.0²,⁶]dodeca-1,4,6,8,11-pentaen-4-yl]-3-[4-(morpholin-4-yl)butoxy]naphthalene-2-carboxamide;

N-{3,10-dithia-5,12-diazatricyclo[7.3.0.0²,⁶]dodeca-1,4,6,8,11-pentaen-4-yl}-3-[4-(morpholin-4-yl)butoxy]-[1,1′-biphenyl]-4-carboxamide;

N-{3,10-dithia-5,12-diazatricyclo[7.3.0.0²,⁶]dodeca-1,4,6,8,11-pentaen-4-yl}-6-[4-(morpholin-4-yl)butoxy]naphthalene-2-carboxamide;

N-{3,10-dithia-5,12-diazatricyclo[7.3.0.0²,⁶]dodeca-1,4,6,8,11-pentaen-4-yl}-6-[2-(morpholin-4-yl)ethoxy]-2H-1,3-benzodioxole-5-carboxamide;

N-{3,10-dithia-5,12-diazatricyclo[7.3.0.0²,⁶]dodeca-1,4,6,8,11-pentaen-4-yl}-6-[4-(morpholin-4-yl)butoxy]-2H-1,3-benzodioxole-5-carboxamide;

N-{3,10-dithia-5,12-diazatricyclo[7.3.0.0²,⁶]dodeca-1,4,6,8,11-pentaen-4-yl}-6-[2-(morpholin-4-yl)ethoxy]-1-benzothiophene-5-carboxamide;

N-{3,10-dithia-5,12-diazatricyclo[7.3.0.0²,⁶]dodeca-1,4,6,8,11-pentaen-4-yl}-6-[4-(morpholin-4-yl)butoxy]-1-benzothiophene-5-carboxamide;

[N-(7-hydroxybenzo[1,2-d:3,4-d′]bis(thiazole)-2-yl)-3-(4-morpholinobutoxy)-2-naphthamide hydrochloride];

N-{3,10-Dithia-5,12-diazatricyclo[7.3.0.0^{2,6}]dodeca-1,4,6,8,11-pentaen-4-yl}-3-{2-[ethyl(2-hydroxyethyl)amino]ethoxy}naphthalene-2-carboxamide carboxamide;

N-{3,10-dithia-5,12-diazatricyclo[7.3.0.0²,⁶]dodeca-1,4,6,8,11-pentaen-4-yl}-3-{2-[(2-hydroxyethyl)amino]ethoxy}naphthalene-2-carboxamide;

N-{3,10-Dithia-5,12-diazatricyclo[7.3.0.0^{2,6}]dodeca-1,4,6,8,11-pentaen-4-yl}-3-[2-(piperazin-1-yl)ethoxy]naphthalene-2-carboxamide;

N-{3,10-dithia-5-azatricyclo[7.3.0.0²,⁶]dodeca-1,4,6,8,11-pentaen-4-yl}-3-[4-(morpholin-4-yl)butoxy]naphthalene-2-carboxamide;

3-[4-(morpholin-4-yl)butoxy]-N-{10-oxa-3-thia-5-azatricyclo[7.3.0.0²,⁶]dodeca-1,4,6,8,11-pentaen-4-yl}naphthalene-2-carboxamide;

3-[2-(morpholin-4-yl)ethoxy]-N-{10-oxa-3-thia-5-azatricyclo[7.3.0.0²,⁶]dodeca-1,4,6,8,11-pentaen-4-yl}naphthalene-2-carboxamide;

3-[2-(morpholin-4-yl)ethoxy]-N-{10-oxa-3-thia-5,12-diazatricyclo[7.3.0.0²,⁶]dodeca-1,4,6,8,11-pentaen-4-yl}naphthalene-2-carboxamide;

3-[4-(morpholin-4-yl)butoxy]-N-{10-oxa-3-thia-5,12-diazatricyclo [7.3.0.0²,⁶]dodeca-1,4,6,8,11-pentaen-4-yl}naphthalene-2-carboxamide;

N-{5,12-dithia-3-azatricyclo[7.3.0.0²,⁶]dodeca-1,3,6,8,10-pentaen-11-yl}-3-[4-(morpholin-4-yl)butoxy]naphthalene-2-carboxamide;

N-{5,12-dithia-3-azatricyclo[7.3.0.0²,⁶]dodeca-1,3,6,8,10-pentaen-11-yl}-3-[2-(morpholin-4-yl)ethoxy]naphthalene-2-carboxamide;

3-[4-(morpholin-4-yl)butoxy]-N-{12-oxa-5-thia-3-azatricyclo [7.3.0.0²,⁶]dodeca-1,3,6,8,10-pentaen-11-yl}naphthalene-2-carboxamide;

3-[2-(morpholin-4-yl)ethoxy]-N-{12-oxa-5-thia-3-azatricyclo [7.3.0.0²,⁶]dodeca-1,3,6,8,10-pentaen-11-yl}naphthalene-2-carboxamide;

3-[2-(morpholin-4-yl)ethoxy]-N-{3-oxa-10-thia-5,12-diazatricyclo [7.3.0.0²,⁶]dodeca-1,4,6,8,11-pentaen-4-yl}naphthalene-2-carboxamide;

3-[4-(morpholin-4-yl)butoxy]-N-{3-oxa-10-thia-5,12-diazatricyclo [7.3.0.0²,⁶]dodeca-1,4,6,8,11-pentaen-4-yl}naphthalene-2-carboxamide;

N-{5,12-dithia-3-azatricyclo[7.3.0.0²,⁶]dodeca-1,3,6,8,10-pentaen-11-yl}-3-[2-(morpholin-4-yl)ethoxy]-[1,1′-biphenyl]-4-carboxamide;

N-{5,12-dithia-3-azatricyclo[7.3.0.0²,⁶]dodeca-1,3,6,8,10-pentaen-11-yl}-3-[4-(morpholin-4-yl)butoxy]-[1,1′-biphenyl]-4-carboxamide;

N-{5,12-dithia-3-azatricyclo[7.3.0.0²,⁶]dodeca-1,3,6,8,10-pentaen-11-yl}-6-[2-(morpholin-4-yl)ethoxy]-2H-1,3-benzodioxole-5-carboxamide;

N-{5,12-dithia-3-azatricyclo[7.3.0.0²,⁶]dodeca-1,3,6,8,10-pentaen-11-yl}-6-[2-(morpholin-4-yl)ethoxy]-2H-1,3-benzodioxole-5-carboxamide;

N-{5,12-dithia-3-azatricyclo[7.3.0.0²,⁶]dodeca-1,3,6,8,10-pentaen-11-yl}-6-[2-(morpholin-4-yl)ethoxy]-1-benzothiophene-5-carboxamide;

N-{5,12-dithia-3-azatricyclo[7.3.0.0²,⁶]dodeca-1,3,6,8,10-pentaen-11-yl}-6-[4-(morpholin-4-yl)butoxy]-1-benzothiophene-5-carboxamide;

N-{4-methoxy-5,12-dithia-3-azatricyclo[7.3.0.0²,⁶]dodeca-1,3,6,8,10-pentaen-11-yl}-6-[2-(morpholin-4-yl)ethoxy]-1-benzothiophene-5-carboxamide;

N-{4-methoxy-5,12-dithia-3-azatricyclo[7.3.0.0²,⁶]dodeca-1,3,6,8,10-pentaen-11-yl}-6-[4-(morpholin-4-yl)butoxy]-1-benzothiophene-5-carboxamide;

N-{3,10-dithia-5,12-diazatricyclo[7.3.0.0²,⁶]dodeca-1,4,6,8,11-pentaen-4-yl}-1-methyl-5-[2-(morpholin-4-yl)ethoxy]-1H-indole-6-carboxamide;

N-{5-thia-3,10,12-triazatricyclo[7.3.0.0²,⁶]dodeca-1,3,6,8,11-pentaen-11-yl}naphthalene-2-carboxamide;

6-[2-(morpholin-4-yl)ethoxy]-N-{4-oxo-5,12-dithia-3-azatricyclo[7.3.0.0²,⁶]dodeca-1,6,8,10-tetraen-11-yl}-1-benzothiophene-5-carboxamide; and

3-[2-(morpholin-4-yl)ethoxy]-N-{3-thia-5,10,12-triazatricyclo[7.3.0.0²,⁶]dodeca-1,4,6,8,11-pentaen-4-yl}naphthalene-2-carboxamide;
or a pharmaceutically acceptable salt thereof.

In some embodiments, provided herein is a compound selected from:

3,5-dimethoxy-N-{11-methyl-3,10-dithia-5,12-diazatricyclo[7.3.0.0²,⁶]dodeca-1,4,6,8,11-pentaen-4-yl}benzamide;

4-(diethyl sulfamoyl)-N-{11-methyl-3,10-dithia-5,12-diazatricyclo[7.3.0.0²,⁶]dodeca-1,4,6,8,11-pentaen-4-yl}benzamide;

N-{3,10-dithia-5,12-diazatricyclo[7.3.0.0²,⁶]dodeca-1,4,6,8,11-pentaen-4-yl}-2H-1,3-benzodioxole-5-carboxamide;

N-{11-methyl-3,10-dithia-5,12-diazatricyclo[7.3.0.0²,⁶]dodeca-1,4,6,8,11-pentaen-4-yl}-4-(pentyloxy)benzamide;

4-(dimethylamino)-N-{11-methyl-3,10-dithia-5,12-diazatricyclo[7.3.0.0²,⁶]dodeca-1,4,6,8,11-pentaen-4-yl}benzamide;

4-chloro-N-{3,10-dithia-5,12-diazatricyclo[7.3.0.0²,⁶]dodeca-1,4,6,8,11-pentaen-4-yl}-3-(trifluoromethyl)benzamide;

N-{3,10-dithia-5,12-diazatricyclo[7.3.0.0²,⁶]dodeca-1,4,6,8,11-pentaen-4-yl}-3-(trifluoromethyl)benzamide;

N-{3,10-dithia-5,12-diazatricyclo [7.3.0.0²,⁶]dodeca-1,4,6,8,11-pentaen-4-yl}-3-nitrobenzamide;

N-(3-bromophenyl)-11-methyl-3,10-dithia-5,12-diazatricyclo[7.3.0.0²,⁶]dodeca-1,4,6,8,11-pentaene-4-carboxamide;

N-{3,10-dithia-5,12-diazatricyclo[7.3.0.0²,⁶]dodeca-1,4,6,8,11-pentaen-4-yl}-1-benzothiophene-2-carboxamide;

N-{3,10-dithia-5,12-diazatricyclo[7.3.0.0²,⁶]dodeca-1,4,6,8,11-pentaen-4-yl}-2,1,3-benzothiadiazole-5-carboxamide;

N-{3,10-dithia-5,12-diazatricyclo[7.3.0.0²,⁶]dodeca-1,4,6,8,11-pentaen-4-yl}-5,6,7,8-tetrahydronaphthalene-2-carboxamide;

N-{3,10-dithia-5,12-diazatricyclo[7.3.0.0²,⁶]dodeca-1,4,6,8,11-pentaen-4-yl}-1-benzothiophene-5-carboxamide;

N-{3,10-dithia-5,12-diazatricyclo[7.3.0.0²,⁶]dodeca-1,4,6,8,11-pentaen-4-yl}-1-benzofuran-5-carboxamide;

N-{3,10-dithia-5,12-diazatricyclo[7.3.0.0²,⁶]dodeca-1,4,6,8,11-pentaen-4-yl}-3-methoxynaphthalene-2-carboxamide;

N-{3,10-dithia-5,12-diazatricyclo[7.3.0.0²,⁶]dodeca-1,4,6,8,11-pentaen-4-yl}-1-methyl-1H-indole-2-carboxamide;

N-{11-ethyl-3,10-dithia-5,12-diazatricyclo[7.3.0.0²,⁶]dodeca-1,4,6,8,11-pentaen-4-yl}naphthalene-2-carboxamide;

N-[11-(methylsulfanyl)-3,10-dithia-5,12-diazatricyclo[7.3.0.0²,⁶]dodeca-1,4,6,8,11-pentaen-4-yl]naphthalene-2-carboxamide;

N-{3,10-dithia-5,12-diazatricyclo[7.3.0.0²,⁶]dodeca-1,4,6,8,11-pentaen-4-yl}-1-methyl-1H-indole-6-carboxamide;

N-{3,10-dithia-5,12-diazatricyclo[7.3.0.0²,⁶]dodeca-1,4,6,8,11-pentaen-4-yl}-[1,1′-biphenyl]-4-carboxamide;

N-{11-methoxy-3,10-dithia-5,12-diazatricyclo[7.3.0.0²,⁶]dodeca-1,4,6,8,11-pentaen-4-yl}naphthalene-2-carboxamide;

N-{11-methyl-3,10-dithia-5,12-diazatricyclo[7.3.0.0²,⁶]dodeca-1,4,6,8,11-pentaen-4-yl}naphthalene-2-carboxamide;

N-{5,12-dithia-3-azatricyclo[7.3.0.0²,⁶]dodeca-1,3,6,8,10-pentaen-11-yl}-1-methyl-1H-indole-2-carboxamide; and

N-{5,12-dithia-3-azatricyclo[7.3.0.0²,⁶]dodeca-1,3,6,8,10-pentaen-11-yl}-1-methyl-1H-indole-2-carboxamide;

or a pharmaceutically acceptable salt thereof.

In some embodiments, provided herein is the following compound:

N-{3,10-dithia-5,12-diazatricyclo[7.3.0.0²,⁶]dodeca-1,4,6,8,11-pentaen-4-yl}-3-[2-(morpholin-4-yl)ethoxy]naphthalene-2-carboxamide;

or a pharmaceutically acceptable salt thereof.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “substituted” means that an atom or group of atoms formally replaces hydrogen as a “substituent” attached to another group. The hydrogen atom is formally removed and replaced by a substituent. A single divalent substituent, e.g., oxo, can replace two hydrogen atoms. The term “optionally substituted” means unsubstituted or substituted. The substituents are independently selected, and substitution may be at any chemically accessible position. It is to be understood that substitution at a given atom is limited by valency. Throughout the definitions, the term “Cᵢ-Cⱼ” or “Cᵢ₋ⱼ” indicates a range which includes the endpoints, wherein i and j are integers and indicate the number of carbons. Examples include C₁-C₄, C₁-C₆, and the like.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1, 2, 3, 4-tetrahydro-naphthalene is an example of a 10-membered cycloalkyl group.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, the term “alkyl,” employed alone or in combination with other terms, refers to a saturated hydrocarbon group that may be linear, or branched, having i to j carbon atoms. In some embodiments, the alkyl group contains from 1 to 10, 1 to 6, 1 to 4, or from 1 to 3 carbon atoms. Examples of alkyl moieties include, but are not limited to, chemical groups such as methyl, ethyl, n-propyl, isopropyl, n-butyl, s-butyl, and t-butyl.

As used herein, “alkenyl,” employed alone or in combination with other terms, refers to an alkyl group having one or more carbon-carbon double bonds. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some embodiments, the alkynyl moiety contains 2 to 6 or 2 to 4 carbon atoms.

As used herein, the term “alkoxy,” employed alone or in combination with other terms, refers to a group of formula —O-alkyl. Example alkoxy groups include methoxy, ethoxy, and propoxy (e.g., n-propoxy and isopropoxy). In some embodiments, the alkyl group has 1 to 3 carbon atoms or 1 to 4 carbon atoms.

As used herein, “haloalkoxy,” employed alone or in combination with other terms, refers to a group of formula —O-(haloalkyl). In some embodiments, the alkyl group has 1 to 6 or 1 to 4 carbon atoms. An example haloalkoxy group is —OCF₃.

As used herein, “amino,” employed alone or in combination with other terms, refers to NH₂.

As used herein, the term “alkylamino”, employed alone or in combination with other terms, refers to a group of formula —NH(alkyl). In some embodiments, the alkyl group has 1 to 6 or 1 to 4 carbon atoms.

As used herein, the term “dialkylamino”, employed alone or in combination with other terms, refers to a group of formula —N(alkyl)₂. In some embodiments, each alkyl group independently has 1 to 6 or 1 to 4 carbon atoms.

As used herein, the term “thio”, employed alone or in combination with other terms, refers to a group of formula —SH.

As used herein, the term “alkylthio”, employed alone or in combination with other terms, refers to a group of formula —S-alkyl. In some embodiments, the alkyl group has 1 to 6 or 1 to 4 carbon atoms.

As used herein, the term “halo”, employed alone or in combination with other terms, refers to a halogen atom selected from F, Cl, I or Br. In some embodiments, “halo” refers to a halogen atom selected from F, Cl, or Br. In some embodiments, the halo group is F.

As used herein, the term “haloalkyl,” employed alone or in combination with other terms, refers to an alkyl group having from one halogen atom to 2s+1 halogen atoms which may be the same or different, where “s” is the number of carbon atoms in the alkyl group. In some embodiments, the haloalkyl group is fluoromethyl, difluoromethyl, or trifluoromethyl. In some embodiments, the haloalkyl group is trifluoromethyl. In some embodiments, the haloalkyl group has 1 to 6 or 1 to 4 carbon atoms.

As used herein the term “aryl”, employed alone or in combination with other terms, has the broadest meaning generally understood in the art, and can include an aromatic ring or aromatic ring system. An aryl group can be monocyclic, bicyclic or polycyclic, and may optionally include one to three additional ring structures; such as, for example, a cycloalkyl, a cycloalkenyl, a heterocycloalkyl, a heterocycloalkenyl, or a heteroaryl. The term “aryl” includes, without limitation, phenyl (benzenyl), naphthyl, tolyl, xylyl, anthracenyl, phenanthryl, azulenyl, biphenyl, naphthalenyl, 1-methylnaphthalenyl, acenaphthenyl, acenaphthylenyl, anthracenyl, fluorenyl, phenalenyl, phenanthrenyl, benzo[a]anthracenyl, benzo[c]phenanthrenyl, chrysenyl, fluoranthenyl, pyrenyl, tetracenyl (naphthacenyl), triphenylenyl, anthanthrenyl, benzopyrenyl, benzo[a]pyrenyl, benzo[e]fluoranthenyl, benzo[ghi]perylenyl, benzo[j]fluoranthenyl, benzo[k]fluoranthenyl, corannulenyl, coronenyl, dicoronylenyl, helicenyl, heptacenyl, hexacenyl, ovalenyl, pentacenyl, picenyl, perylenyl, and tetraphenylenyl. In some embodiments, aryl is C₆₋₁₀ aryl. In some embodiments, the aryl group is a naphthalene ring or phenyl ring. In some embodiments, the aryl group is phenyl. In other embodiments, the aryl group is a naphthyl.

As used herein, the term “arylalkyl,” employed alone or in combination with other terms, refers to a group of formula aryl-alkyl-. In some embodiments, the alkyl portion has 1 to 4, 1 to 3, 1 to 2, or 1 carbon atom(s). In some embodiments, the alkyl portion is methylene. In some embodiments, the aryl portion is phenyl. In some embodiments, the aryl group is a monocyclic or bicyclic group. In some embodiments, the arylalkyl group is benzyl.

As used herein, the term “heteroaryl,” employed alone or in combination with other terms, refers to a monocyclic or polycyclic (e.g., having 2, 3 or 4 fused rings) aromatic moiety, having one or more heteroatom ring members selected from nitrogen, sulfur and oxygen. In some embodiments, the heteroaryl group is a 5- to 10-membered heteroaryl ring, which is monocyclic or bicyclic and which has 1, 2, 3, or 4 heteroatom ring members independently selected from nitrogen, sulfur and oxygen. In some embodiments, the heteroaryl group is a 5- to 6-membered heteroaryl ring, which is monocyclic and which has 1, 2, 3, or 4 heteroatom ring members independently selected from nitrogen, sulfur and oxygen. When the heteroaryl group contains more than one heteroatom ring member, the heteroatoms may be the same or different. The nitrogen atoms in the ring(s) of the heteroaryl group can be oxidized to form N-oxides. Example heteroaryl groups include, but are not limited to, pyridine, pyrimidine, pyrazine, pyridazine, pyrrole, pyrazole, azolyl, oxazole, thiazole, imidazole, furan, thiophene, quinoline, isoquinoline, indole, benzothiophene, benzofuran, benzisoxazole, imidazo[1,2-b]thiazole, purine, and the like.

A 5-membered heteroaryl”, employed alone or in combination with other terms, is a heteroaryl group having five ring-forming atoms comprising carbon and one or more (e.g., 1, 2, or 3) ring atoms independently selected from N, 0, and S. Example five-membered heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered heteroary”, employed alone or in combination with other terms, 1 is a heteroaryl group having six ring-forming atoms wherein one or more (e.g., 1, 2, or 3) ring atoms are independently selected from N, 0, and S. Example six-membered heteroaryls include pyridyl, pyrazinyl, pyrimidinyl, triazinyl and pyridazinyl.

As used herein, the term “heteroarylalkyl,” employed alone or in combination with other terms, refers to a group of formula heteroaryl-alkyl-. In some embodiments, the alkyl portion has 1 to 4, 1 to 3, 1 to 2, or 1 carbon atom(s). In some embodiments, the alkyl portion is methylene. In some embodiments, the heteroaryl portion is a monocyclic or bicyclic group having 1, 2, 3, or 4 heteroatoms independently selected from nitrogen, sulfur and oxygen. In some embodiments, the heteroaryl portion has 5 to 10 carbon atoms. In some embodiments, the heteroaryl portion is a 5-10 membered heteroaryl ring.

As used herein, the term “cycloalkyl,” employed alone or in combination with other terms, refers to a non-aromatic cyclic hydrocarbon including cyclized alkyl and alkenyl groups. Cycloalkyl groups can include mono- or polycyclic (e.g., having 2, 3, or 4 fused, bridged, or spiro rings) ring systems. Also included in the definition of cycloalkyl are moieties that have one or more aromatic rings (e.g., aryl or heteroaryl rings) fused (i.e., having a bond in common with) to the cycloalkyl ring, for example, benzo derivatives of cyclopentane, cyclohexene, cyclohexane, and the like, or pyrido derivatives of cyclopentane or cyclohexane. Ring-forming carbon atoms of a cycloalkyl group can be optionally substituted by oxo. Cycloalkyl groups also include cycloalkylidenes. The term “cycloalkyl” also includes bridgehead cycloalkyl groups (e.g., non-aromatic cyclic hydrocarbon moieties containing at least one bridgehead carbon, such as admantan-1-yl) and spirocycloalkyl groups (e.g., non-aromatic hydrocarbon moieties containing at least two rings fused at a single carbon atom, such as spiro[2.5]octane and the like). In some embodiments, the cycloalkyl group has 3 to 10 ring members, or 3 to 7 ring members. In some embodiments, the cycloalkyl group is monocyclic or bicyclic. In some embodiments, the cycloalkyl group is monocyclic. In some embodiments, the cycloalkyl group is a C₃₋₇ monocyclic cycloalkyl group. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, tetrahydronaphthalenyl, octahydronaphthalenyl, indanyl, and the like. In some embodiments, the cycloalkyl group is cyclopropyl, cyclobutyl, cyclopentyl, or cyclohexyl.

As used herein, the term “cycloalkylalkyl,” employed alone or in combination with other terms, refers to a group of formula cycloalkyl-alkyl-. In some embodiments, the alkyl portion has 1 to 4, 1 to 3, 1 to 2, or 1 carbon atom(s). In some embodiments, the alkyl portion is methylene. In some embodiments, the cycloalkyl portion has 3 to 10 ring members or 3 to 7 ring members. In some embodiments, the cycloalkyl group is monocyclic or bicyclic. In some embodiments, the cycloalkyl portion is monocyclic. In some embodiments, the cycloalkyl portion is a C₃₋₇ monocyclic cycloalkyl group. In some embodiments, the cycloalkylalkyl group is cyclopentylmethyl.

As used herein, the term “heterocycloalkyl,” employed alone or in combination with other terms, refers to a non-aromatic ring or ring system, which may optionally contain one or more alkynylene or alkynylene groups as part of the ring structure, which has at least one heteroatom ring member independently selected from nitrogen, sulfur, oxygen, and phosphorus. Heterocycloalkyl groups can include mono- or polycyclic (e.g., having 2, 3 or 4 fused, bridged, or spiro rings) ring systems. In some embodiments, the heterocycloalkyl group is a monocyclic or bicyclic group having 1, 2, 3, or 4 heteroatoms independently selected from nitrogen, sulfur and oxygen. Also included in the definition of heterocycloalkyl are moieties that have one or more aromatic rings (e.g., aryl or heteroaryl rings) fused (i.e., having a bond in common with) to the non-aromatic heterocycloalkyl ring, for example, 1,2,3,4-tetrahydro-quinoline and the like. Heterocycloalkyl groups can also include bridgehead heterocycloalkyl groups (e.g., a heterocycloalkyl moiety containing at least one bridgehead atom, such as azaadmantan-1-yl and the like) and spiroheterocycloalkyl groups (e.g., a heterocycloalkyl moiety containing at least two rings fused at a single atom, such as [1,4-dioxa-8-aza-spiro[4.5]decan-N-yl] and the like). In some embodiments, the heterocycloalkyl group has 3 to 10 ring-forming atoms, 4 to 10 ring-forming atoms, or about 3 to 8 ring forming atoms. In some embodiments, the heterocycloalkyl group has 2 to 20 carbon atoms, 2 to 15 carbon atoms, 2 to 10 carbon atoms, or about 2 to 8 carbon atoms. In some embodiments, the heterocycloalkyl group has 1 to 5 heteroatoms, 1 to 4 heteroatoms, 1 to 3 heteroatoms, or 1 to 2 heteroatoms. The carbon atoms or heteroatoms in the ring(s) of the heterocycloalkyl group can be oxidized to form a carbonyl, an N-oxide, or a sulfonyl group (or other oxidized linkage) or a nitrogen atom can be quaternized. In some embodiments, the heterocycloalkyl group is a morpholine ring, pyrrolidine ring, piperazine ring, piperidine ring, tetrahydropyran ring, tetrahyropyridine, azetidine ring, or tetrahydrofuran ring.

As used herein, the term “heterocycloalkylalkyl,” employed alone or in combination with other terms, refers to a group of formula heterocycloalkyl-alkyl-. In some embodiments, the alkyl portion has 1 to 4, 1 to 3, 1 to 2, or 1 carbon atom(s). In some embodiments, the alkyl portion is methylene. In some embodiments, the heterocycloalkyl portion has 3 to 10 ring members, 4 to 10 ring members, or 3 to 7 ring members. In some embodiments, the heterocycloalkyl group is monocyclic or bicyclic. In some embodiments, the heterocycloalkyl portion is monocyclic. In some embodiments, the heterocycloalkyl portion is a 4-7 membered monocyclic heterocycloalkyl group.

The compounds described herein can be asymmetric (e.g., having one or more stereocenters). All stereoisomers, such as enantiomers and diastereoisomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention may be isolated as a mixture of isomers or as separated isomeric forms.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as α-camphorsulfonic acid. Other resolving agents suitable for fractional crystallization methods include stereoisomerically pure forms of α-methylbenzylamine (e.g., S and R forms, or diastereoisomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention can also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole.

Compounds of the invention can also include all isotopes of atoms occurring in the intermediates or final compounds. Isotopes include those atoms having the same atomic number but different mass numbers. For example, isotopes of hydrogen include tritium and deuterium. In some embodiments, the compounds of the invention each contain at least one deuterium.

The term “compound,” as used herein, is meant to include all stereoisomers, geometric isomers, tautomers, and isotopes of the structures depicted. Compounds herein identified by name or structure as one particular tautomeric form are intended to include other tautomeric forms unless otherwise specified. Compounds herein identified by name or structure without specifying the particular configuration of a stereocenter are meant to encompass all the possible configurations at the stereocenter. For example, if a particular stereocenter in a compound of the invention could be R or S, but the name or structure of the compound does not designate which it is, then the stereocenter can be either R or S.

All compounds, and pharmaceutically acceptable salts thereof, can be found together with other substances such as water and solvents (e.g., hydrates and solvates) or can be isolated.

In some embodiments, the compounds of the invention, or salts thereof, are substantially isolated. By “substantially isolated” is meant that the compound is at least partially or substantially separated from the environment in which it was formed or detected. Partial separation can include, for example, a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “RT,” as used herein, are understood in the art, and refer generally to a temperature, e.g., a reaction temperature, that is about the temperature of the room in which the reaction is carried out, for example, a temperature from about 20° C. to about 30° C.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (CH₃CN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19, and in Stahl et al., Handbook of Pharmaceutical Salts: Properties, Selection, and Use, (Wiley, 2002).

The below list is a key to abbreviations that may be used throughout.

### Abbreviations

Abbreviation Definition
AcOH Acetic acid
ALK5 Activin Receptor-Like Kinase Receptor 5
BTLA B and T lymphocyte attenuator
(Boc)₂O Di-tert-butyl dicaronate
CAS Chemical Abstract Service registry number
CCR Chemokine receptor type
CTLA4 Cytotoxic T lymphocyte associated protein 4
DIAD Diisopropyl azodicarboxylate
DCM Dichloromethane
DIPEA N,N-diisopropylethylamine
DMF Dimethyl formamide
DMSO Dimethyl sulfoxide
DPPA Diphenylphosphoryl azide
EtOAc Ethyl acetate
FBS Fetal bovine serum
Fe Iron
H Hour(s)
HA hemagglutination assay
HATU 1-[Bis(dimethylamino)methylene]-1H-1,2,3-triazolo[4,5-b]pyridinium 3-oxid hexafluorophosphate
Hex Hexanes
KIR Killer cell immunoglobulin-like receptor
LAG3 Lymphocyte activation gene 3
Min Minute(s)
mL Milliliter(s)
HPLC High-performance liquid chromatography
ICD Immunogenic Cell Death
IFN Interferon
IRF3 Interferon regulatory transcription factor (IRF) family 3
ISG IFN-stimulated genes
IPA Isopropyl alcohol
LC/MS Liquid chromatography/mass spectrometry
LiOH Lithium hydroxide
MeOH Methanol
MS Mass spectrometry
MTBE Methyl test-butyl ether
NaH Sodium hydride
NMP N-Methyl-2-pyrrolidone
PDL Programmed death ligand
PDGFR-2 Plasminogen-related growth factor receptor 2
PMA Phorbol 12-myristate 13-acetate
RLR RIG-I-like receptor
RPMI Roswell park memorial institute medium
RT Room Temperature
t-BuOH Tert-Butanol
TBTU O-(benzotriazol-1-yl)-N,N,N′,N′-tetramethyluronium tetrafluoroborate
TEA Triethylamine
TFA trifluoroacetic acid
THF Tetrahydrofuran
TIM3 T cell immunoglobulin and mucin domain 3
TLR Toll-like receptor
U Units
uM Micromolar
VISTA V-domain Ig suppressor of T cell activation

### Synthesis

Procedures for making compounds described herein are provided below with reference to Scheme 1. Optimum reaction conditions and reaction times may vary depending on the particular reactants used. Unless otherwise specified, solvents, temperatures, pressures and other reaction conditions are readily selected by one of ordinary skill in the art. Specific procedures are provided in the Examples section. Compounds are named using the “structure to name” function included in MarvinSketch 5.9.0.

Typically, reaction progress may be monitored by thin layer chromatography (TLC) or HPLC-MS if desired. Intermediates and products may be purified by chromatography on silica gel, recrystallization, HPLC and/or reverse phase HPLC. In the reactions described below, it may be necessary to protect reactive functional groups (such as hydroxy, amino, thio, or carboxy groups) to avoid their unwanted participation in the reactions. The incorporation of such groups, and the methods required to introduce and remove them are known to those skilled in the art (for example, see Greene, Wuts, Protective Groups in Organic Synthesis. 2nd Ed. (1999)). One or more deprotection steps in the synthetic schemes may be required to ultimately afford compounds of Formula I. The protecting groups depicted in the schemes are used as examples, and may be replaced by other compatible alternative groups. Starting materials used in the following schemes can be purchased or prepared by methods described in the chemical literature, or by adaptations thereof, using methods known by those skilled in the art. The order in which the steps are performed can vary depending on the protecting or functional groups introduced and the reagents and reaction conditions used, but would be apparent to those skilled in the art.

Compounds of the invention, such as benzobisthiazole compounds, can be prepared as shown in Scheme 1. 2-Chloro-5-nitroaniline (A) can be heated with formic acid to provide the formamide intermediate (B). Cyclization with sodium sulfide in a solvent (e.g., ethanol) under heating provides 5-nitrobenzo[d]thiazole (C). Nitro-reduction with iron in an acidic solvent (e.g., acetic acid) under heating provides benzo[d]thiazol-5-amine (D). Treatment with ammonium thiocyanate in the presence of Br₂ provides benzo[1,2-d:3,4-d′]bis(thiazole)-2-amine (E).

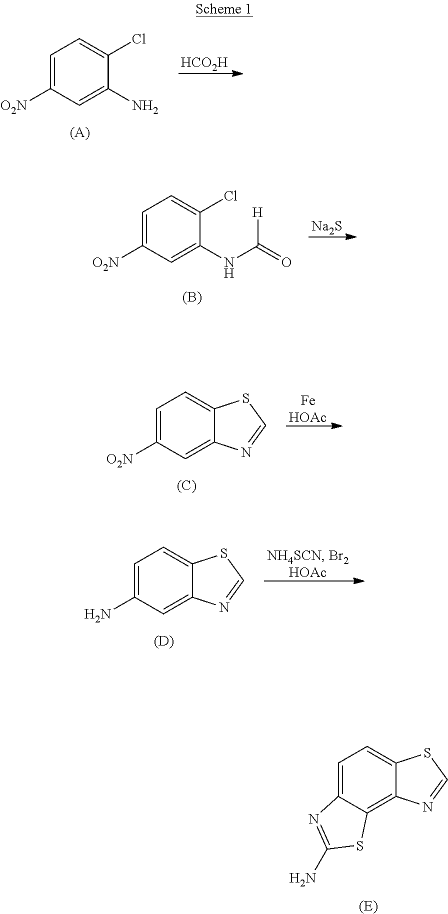

Alternative cores to the benzobisthiazole core can generally be prepared as described in Scheme 2. An aromatic substituted aldehlyde (F) and methyl 2-mercaptoacetate are heated in a solvent (e.g., DMF) to provide the 7-nitrobenzo[b]thiophene-2-carboxylate (G). The nitro group of compound (G) is then reduced under appropriate reducing conditions (e.g. Fe in acetic acid) to provide methyl 7-aminobenzo[b]thiophene-2-carboxylate (H). Reaction of compound (H) with benzoyl isothiocyanate in a solvent (e.g., acetonitrile) provides methyl 7-(3-benzoylthioureido) benzo[b]thiophene-2-carboxylate (I). Hydrolysis of the thiouredido and carboxylate groups of compound (I) with a base (e.g., sodium hydroxide) in a solvent (e.g., methanol) provides the thiourea (J). Cyclization of the benzo[1,2-d]thiazole is accomplished by treatment with bromine to provide carboxylic acid (K). Curtius rearrangement of intermediate (K) by treatment with DPPA in the presence of tert-butanol provides the carbamate (L). Deprotection of the carbamate (L) with acid (e.g., HCl) provides the benzobisthiazole compound (M).

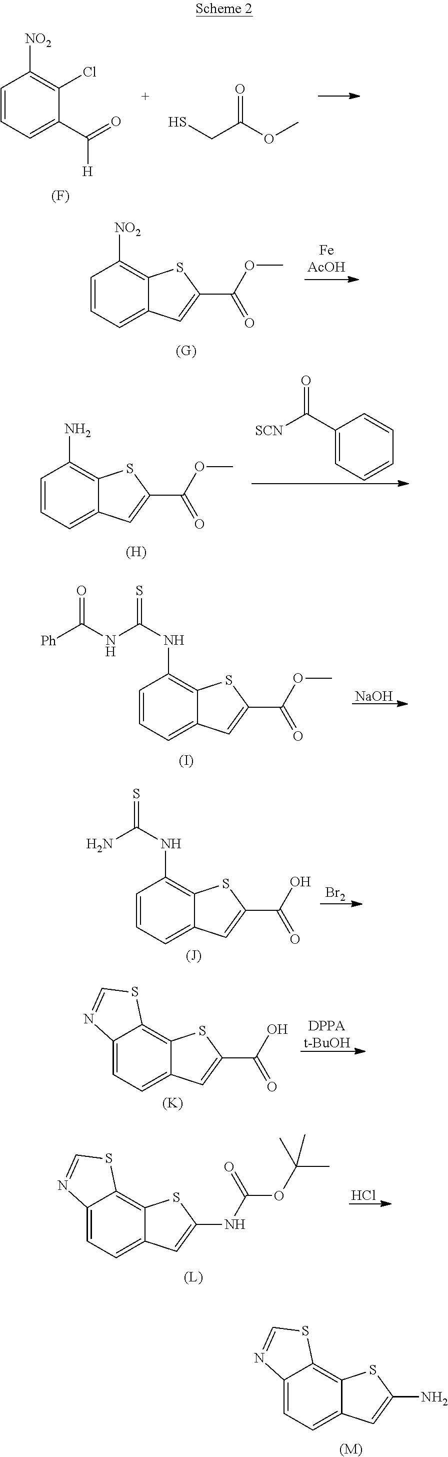

Substituted aromatic carboxylic acids can be produced as shown in Scheme 3. An appropriately substituted hydroxy substituted carboxylic acid (N) can be treated with an amino halide (X═Cl or Br; j is 2, 3, 4, 5, or 6) in a solvent (e.g., DMF) in the presence of a base (e.g., Cs₂CO₃) to provide the ether product (O).

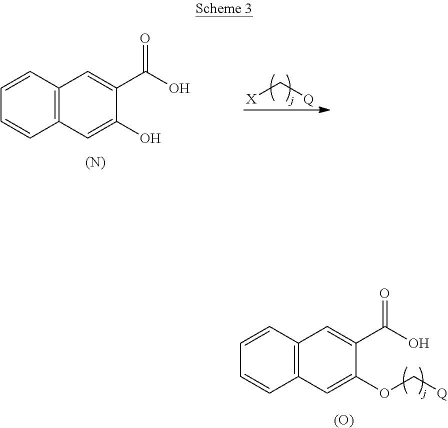

Amides can be produced from an amine intermediate and a carboxylic acid intermediate, as shown in Scheme 4. Amine (P) can be coupled with a carboxylic acid (O) using standard peptide coupling reagents (e.g. HATU, DIPEA) in a solvent (e.g., DMF) to provide amide (Q).

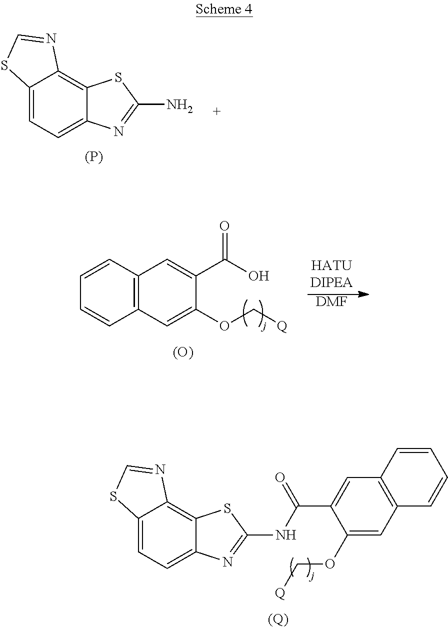

### Methods

The present disclosure provides methods of agonizing the retinoic acid-inducible gene-I pathway by contacting RIG-I with a compound of the invention, or a pharmaceutically acceptable salt thereof. In some embodiments, the invention provides methods for inducing the expression of cytokines or chemokines associated with the RIG-1 pathway. Cytokines or chemokinates that are associated with the RIG-I pathway can include, for example, interferon sensitive response element (ISRE), proinflammatory cytokines, RANTES, and CXCL10.

The present disclosure further provides methods for activating interferon regulatory factor 3 (IRF3) by contacting IRF3 with a compound of the invention, or a pharmaceutically acceptable salt thereof. The activation of IRF3 can result in the expression of IRF3-dependent genes. In some embodiments, the expression of IRF3-dependent genes is induced by a factor of about 1 to about 40-fold. In some embodiments, the expression of IRF3-dependent genes is induced by a factor in the range of about 10 to about 20-fold, about 20 to about 40-fold, or greater than about 40-fold. In some embodiments, the expression of CXCL-10 (IP-10) is induced, resulting in an increase in concentration of CXCL-10. In some embodiments, the expression of CXCL-10 is induced to a concentration of CXCL-10 that is greater than about 1,600 pg/mL. In some embodiments, the expression of CXCL-10 (IP-10) is induced to a concentration of CXCL-10 that is about 400 pg/mL to about 800 pg/mL, to about 800 pg/mL to about 1,600 pg/mL, or greater than about 1,600 pg/mL. In some embodiments, the induction of expression of IRF3 occurs within about 24 h following administration of a compound described herein or a pharmaceutically acceptable salt thereof. In some embodiments, the compounds described herein induce the expression of CXCL10 in cancer cells. In some embodiments, the cancer cells are colon carcinoma cells. In some embodiments, the compounds described herein stimulate the release of DAMPs.

In some embodiments, the contacting can be administering to a patient a compound provided herein, or a pharmaceutically acceptable salt thereof. In certain embodiments, the compounds of the present disclosure, or pharmaceutically acceptable salts thereof, are useful for therapeutic administration to enhance, stimulate and/or increase immunity in cancer. For example, a method of treating a disease or disorder can include administering to a patient in need thereof a therapeutically effective amount of a compound provided herein, or a pharmaceutically acceptable salt thereof. The compounds of the present disclosure can be used alone, in combination with other agents or therapies or as an adjuvant or neoadjuvant for the treatment of diseases or disorders, including cancers. For the uses described herein, any of the compounds of the disclosure, including any of the embodiments thereof, may be used.

Diseases and disorders that a treatable using compounds of the present disclosure include, but are not limited to, cell-proliferation disorders and immune-related diseases. In some embodiments, the cell-proliferation disorder is cancer, benign papillomatosis, a gestational trophoblastic disease, or a benign neoplastic disease (e.g., skin papilloma [warts] and genital papilloma). In some embodiments, the cell-proliferation disorder is a cancer.

Examples of cancers that are treatable using compounds of the present disclosure include, but are not limited to, brain cancer, cancer of the spine, cancer of the head, cancer of the neck, leukemia, blood cancers, cancer of the reproductive system, gastrointestinal cancer, liver cancer, bile duct cancer, kidney cancer, bladder cancer, bone cancer, lung cancer, malignant mesothelioma, sarcomas, lymphomas, glandular cancer, thyroid cancer, heart cancer, malignant neuroendocrine (carcinoid) tumors, midline tract cancers, and metastasized cancers.

In specific embodiments, cancers of the brain and spine include anaplastic astrocytomas, glioblastomas, astrocytomas, and estheosioneuroblastomas (also known as olfactory blastomas). In particular embodiments, the brain cancer includes astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma), oligodendroglial tumor (e.g., oligodendroglioma, and anaplastic oligodendroglioma), oligoastrocytic tumor (e.g., oligoastrocytoma, and anaplastic oligoastrocytoma), ependymoma (e.g., myxopapillary ependymoma, and anaplastic ependymoma); medulloblastoma, primitive neuroectodermal tumor, schwannoma, meningioma, atypical meningioma, anaplastic meningioma, pituitary adenoma, brain stem glioma, cerebellar astrocytoma, cerebral astorcytoma/malignant glioma, visual pathway and hypothalmic glioma, and primary central nervous system lymphoma. In specific instances of these embodiments, the brain cancer is selected from the group consisting of glioma, glioblastoma multiforme, paraganglioma, and suprantentorial primordial neuroectodermal tumors (sPNET).

In specific embodiments, cancers of the head and neck include nasopharyngeal cancers, nasal cavity and paranasal sinus cancers, hypopharyngeal cancers, oral cavity cancers (e.g., squamous cell carcinomas, lymphomas, and sarcomas), lip cancers, oropharyngeal cancers, salivary gland tumors, cancers of the larynx (e.g., laryngeal squamous cell carcinomas, rhabdomyosarcomas), and cancers of the eye or ocular cancers (e.g., intraocular melanoma and retinoblastoma).

In specific embodiments, leukemia and cancers of the blood include myeloproliferative neoplasms, myelodysplastic syndromes, myelodysplastic/myeloproliferative neoplasms, acute myeloid leukemia (AML), myelodysplastic syndrome (MDS), chronic myelogenous leukemia (CML), myeloproliferative neoplasm (MPN), post-MPN AML, post-MDS AML, del(5q)-associated high risk MDS or AML, blastphase chronic myelogenous leukemia, angioimmunoblastic lymphoma, acute lymphoblastic leukemia, Langerans cell histiocytosis, hairy cell leukemia, and plasma cell neoplasms including plasmacytomas and multiple myelomas. Leukemias referenced herein may be acute or chronic

In specific embodiments, skin cancers include melanoma, squamous cell cancers, and basal cell cancers.

In specific embodiments, reproductive system cancers include breast cancers, cervical cancers, vaginal cancers, ovarian cancers, prostate cancers, penile cancers, and testicular cancers. In specific instances of these embodiments, breast cancer includes ductal carcinomas and phyllodes tumors. In specific instances of these embodiments, the breast cancer may be male breast cancer or female breast cancer. In specific instances of these embodiments, cervical cancer includes squamous cell carcinomas and adenocarcinomas. In specific instances of these embodiments, the cancer is an ovarian cancer selected from the group consisting of epithelial cancers.

In specific embodiments, gastrointestinal cancers include esophageal cancers, gastric cancers (also known as stomach cancers), gastrointestinal carcinoid tumors, pancreatic cancers, gallbladder cancers, colorectal cancers, and anal cancer, and can include esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gallbladder adenocarcinomas, colorectal adenocarcinomas, and anal squamous cell carcinomas.

In specific embodiments, the liver cancer is hepatocellular carcinoma.

In particular embodiments, the cancer is bile duct cancer (also known as cholangiocarcinoma) including intrahepatic cholangiocarcinoma and extrahepatic cholangiocarcinoma.

In specific embodiments, kidney and bladder cancers include renal cell cancer, Wilms tumors, and transitional cell cancers. In particular embodiments, the cancer is a bladder cancer, including urethelial carcinoma (a transitional cell carcinoma), squamous cell carcinomas, and adenocarcinomas.

In specific embodiments, bone cancers include osteosarcoma, malignant fibrous histiocytoma of bone, Ewing sarcoma, and chordoma (cancer of the bone along the spine).

In specific embodiments, lung cancers include non-small cell lung cancer, small cell lung cancers, bronchial tumors, and pleuropulmonary blastomas.

In specific embodiments, the cancer is selected from malignant mesothelioma, consisting of epithelial mesothelioma and sarcomatoids.

In specific embodiments, sarcomas include central chondrosarcoma, central and periosteal chondroma, fibrosarcoma, clear cell sarcoma of tendon sheaths, and Kaposi's sarcoma.

In specific embodiments, lymphoma cancers include Hodgkin lymphoma (e.g., Reed-Sternberg cells), non-Hodgkin lymphoma (e.g., diffuse large B-cell lymphoma, follicular lymphoma, mycosis fungoides, Sezary syndrome, primary central nervous system lymphoma), cutaneous T-cell lymphomas, primary central nervous system lymphomas.

In specific embodiments, glandular cancers include adrenocortical cancer (also known as adrenocortical carcinoma or adrenal cortical carcinoma), pheochromocytomas, paragangliomas, pituitary tumors, thymoma, and thymic carcinomas.

In specific embodiments, thyroid cancers include medullary thyroid carcinomas, papillary thyroid carcinomas, and follicular thyroid carcinomas.

In specific embodiments, the cancer is selected from germ cell tumors, include malignant extracranial germ cell tumors and malignant extragonadal germ cell tumors. In specific instances of these embodiments, the malignant extragonadal germ cell tumors include nonseminomas and seminomas.

In specific embodiments, heart tumor cancers include malignant teratoma, lymphoma, rhabdomyosacroma, angiosarcoma, chondrosarcoma, infantile fibrosarcoma, and synovial sarcoma.

In certain other embodiments, the methods include, but are not limited to, administering a compound described herein to a subject in order to induce immunogenic cell death of cancer cells (e.g., tumor cells). In other embodiments, the methods include but are not limited to administering the compound to induce T cell responses including memory T cell responses specific to cancer antigens.

In further aspects, the invention provides methods for inducing an innate immune response in a subject, comprising administering a therapeutically effective amount of a compound described herein or a pharmaceutically acceptable salt thereof. In certain embodiments, the subject is a human.

The present disclosure also includes the following embodiments:

a compound of Formula I, or a pharmaceutically acceptable salt thereof, as defined in any of the embodiments described herein, for use as a medicament;

a compound of Formula I, or a pharmaceutically acceptable salt thereof, as defined in any of the embodiments described herein, for use in the treatment of the hereinabove-mentioned indications; and

a compound of Formula I, or a pharmaceutically acceptable salt thereof, as defined in any of the embodiments described herein, for use in the treatment of a cell proliferation disorder, such as cancer;

the use of a compound of Formula I, or a pharmaceutically acceptable salt thereof, as defined in any of the embodiments described herein, for the manufacture of a medicament for treating a disease or condition for which an activator of the RIG-I pathway is indicated;

a compound of Formula I, or a pharmaceutically acceptable salt thereof, as defined in any of the embodiments described herein, for use in the treatment of a disease or condition for which an activator of the RIG-I pathway is indicated; and

a pharmaceutical composition for the treatment of a disease or condition for which an activator of the RIG-I pathway is indicated, comprising a compound of Formula I, or a pharmaceutically acceptable salt thereof, as defined in any of the embodiments described herein

As used herein, the term “contacting” refers to the bringing together of the indicated moieties in an in vitro system or an in vivo system such that they are in sufficient physical proximity to interact.

The terms “individual” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

As used herein, the term “prophylactic” refers to preventing the disease, i.e. causing the clinical symptoms or signs of the disease not to develop in a subject, such as a mammal that may be exposed to or predisposed to the disease but does not yet experience or display symptoms/signs of the disease.

### Combination Therapy

The compounds of the present disclosure can be administered with one or more additional therapeutic agents. In certain embodiments, the one or more therapeutic agents include an immune stimulator, including but not limited to a stimulator of T cells or dendritic cells. The one or more therapeutic agents can be selected from, inter alia, the group consisting of adjuvants, CTLA-4 and PD-I pathway antagonists and other immunomodulatory agents, lipids, liposomes, peptides, anti-cancer and chemotherapeutic agents.

The CLTA-4 and PD-I pathways are important negative regulators of immune response. Activated T-cells up-regulate CTLA-4, which binds on antigen-presenting cells and inhibits T-cell stimulation, IL-2 gene expression, and T-cell proliferation; these anti-tumor effects have been observed in mouse models of colon carcinoma, metastatic prostate cancer, and metastatic melanoma. PD-I binds to active T-cells and suppresses T-cell activation; PD-I antagonists have demonstrated anti-tumor effects as well. CTLA-4 and PD-I pathway antagonists that may be used in combination with the compounds described herein, or the pharmaceutically acceptable salts thereof, include ipilimumab, tremelimumab, nivolumab, pembrolizumab, CT-011, AMP-224, and MDX1106.

“PD-1 antagonist” or “PD-1 pathway antagonist” refers to any chemical compound or biological molecule that blocks binding of PD-L1 expressed on a cancer cell to PD-I expressed on an immune cell (T-cell, B-cell, or NKT-cell), blocks binding of PD-L2 expressed on a cancer cell to the immune-cell expressed PD-L. Synonyms for PD-L include PD-I: PDCDI, PD1, CD279, and SLEB2 for PD-1; PDCD1L1, PDLI, B7H1, B7-4, CD274, and B7-H for PD-L1; and PDCD1L2, PDL2, B7-DC, Btdc, and CD273 for PD-L2.

Additionally, the use of cytotoxic agents may be used in combination with the compounds described herein, or pharmaceutically acceptable salts thereof, include, but are not limited to, arsenic trioxide (Trisenox®), asparaginase (also known as L-asparaginase, and Erwinia L-asparaginase, Elspar® and Kidrolase®).

Chemotherapeutic agents that may be used in combination with the compounds described herein, or pharmaceutically acceptable salts thereof, include abiraterone acetate, altretamine, anhydrovinblastine, auristatin, bexarotene, bicalutamide, BMS 184476, 2,3,4,5,6-pentafluoro-N-(3-fluoro-4-methoxyphenyl) benzene sulfonamide, bleomycin, N,N-dimethyl-L-valyl-L-valyl-N-methyl-L-valyl-Lprolyl-1-Lproline-t-butylamide, cachectin, cemadotin, chlorambucil, cyclophosphamide, 3′,4′-didehydro-4′-deoxy-8¹-norvin-caleukoblastine, docetaxol, doxetaxel, cyclophosphamide, carboplatin, carmustine, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyurea andtaxanes, ifosfamide, liarozole, lonidamine, lomustine (CCNU), MDV3100, mechlorethamine (nitrogen mustard), melphalan, mivobulin isethionate, rhizoxin, sertenef, streptozocin, mitomycin, methotrexate, taxanes, nilutamide, nivolumab, onapristone, paclitaxel, pembrolizumab, prednimustine, procarbazine, RPR109881, stramustine phosphate, tamoxifen, tasonermin, taxol, tretinoin, vinblastine, vincristine, vindesine sulfate, and vinflunine.

Examples of vascular endothelial growth factor (VEGF) receptor inhibitors that may be used with the compounds described herein include, but are not limited to, bevacizumab (AVASTIN by Genentech/Roche), axitinib, Brivanib Alaninate (BMS-582664), motesanib (SO 230), and sorafenib (NEXAVAR). Such inhibitors may be provided as a pharmaceutically acceptable salt, where appropriate.

Examples of topoisomerase II inhibitors that may be used with the compounds described herein include, but are not limited to, etoposide (also known as VP-16 and Etoposide phosphate, TOPOSAR, VEPESID, and ETOPOPFiOS), and teniposide (VUMON). Such inhibitors may be provided as a pharmaceutically acceptable salt, where appropriate.

Examples of alkylating agents that may be used with the compounds described herein include, but are not limited to, 5-azacytidine (VIDAZA), decitabine (DECOGEN), temozolomide (TEMODAR and TEMODAL), dactinomycin (COSMEGEN), melphalan (ALKERAN), altretamine (FiEXALEN), carmustine (BCNU), bendamustine (TREANDA), busulfan (Busuefex® and Myleran®), carboplatin (Paraplatin®), lomustine (CeeNU®), cisplatin (Platinol® and Platinol®-AQ), chlorambucil (Leukeran®), cyclophosphamide (Cytoxan® and Neosar®), dacarbazine (DTICDome), altretamine (Flexalen®), ifosfamide (Ifex®), procarbazine (Matulane®), mechlorethamine (Mustargen®), streptozocin (Zanosar®), thiotepa (Thioplex®). Such alkylating agents may be provided as a pharmaceutically acceptable salt, where appropriate.

Examples of anti-tumor antibiotics that may be used with the compounds described herein include, but are not limited to, doxorubicin (Adriamycin® and Rubex®), bleomycin (Lenoxane®), daunorubicin (Cerubidine®), daunorubicin liposomal (DaunoXome®), mitoxantrone (Novantrone®), epirubicin (Ellence™), idarubicin (Idamycin®, Idamycin PFS®), and mitomycin C (Mutamycin®). Such anti-tumor antibiotics may be provided as a pharmaceutically acceptable salt, where appropriate.

Examples of anti-metabolites that may be used with the compounds described herein include, but are not limited to, claribine (Leustatin®), 5-fluorouracil (Adrucil®, 6-thioguanine (Purinethol®), pemetrexed (Alimta®), cytarabine (Cytosar-U®), cytarabine liposomal (DepoCyt®), decitabine (Dacogen®), hydroxyurea and (Flydrea®, Droxia™ and Mylocel™) fludarabine (Fludara®), floxuridine (FUDR®), cladribine Leustatin™), methotrexate (Rheumatrex® and Trexall™), and pentostatin (Nipent®). Such anti-metabolites may be provided as a pharmaceutically acceptable salt, where appropriate.

Examples of retinoids that may be used with the compounds described herein include, but are not limited to, alitretinoin (Panretin®), tretinoin (Vesanoid®), Isotretinoin (Accutane®), and bexarotene (Targretin®). Such compounds may be provided as a pharmaceutically acceptable salt, where appropriate.

Immuno-oncology therapy agents (e.g., a checkpoint inhibitor) may also be used in combination with the compounds described herein. Representative immuno-oncology therapy agents include, for example, those targeting the adenosine A2A receptor, Activin Receptor-Like Kinase Receptor 5 (ALK5), BRAF, B7-H3, B7-H4, B and T lymphocyte attenuator (BTLA), cytotoxic T lymphocyte associated protein 4 (CTLA4), CSF1, CXCR2, CXCR4, chemokine receptor type 2 (CCR2), chemokine receptor type 5 (CCR5), indoleamine 2,3-dioxygenase (IDO), killer cell immunoglobulin-like receptor (KIR), lymphocyte activation gene 3 (LAG3), PDE5, plasminogen-related growth factor receptor 2 (PRGFR-2), T cell immunoglobulin and mucin domain 3 (TIM3), or V-domain Ig suppressor of T cell activation (VISTA).

Antigens and adjuvants that may be used in combination with the compounds described herein include B7 costimulatory molecule, interleukin-2, interferon-y, GM-CSF, CTLA-4 antagonists, OX-40/0X-40 ligand, CD40/CD40 ligand, sargramostim, levamisol, vaccinia virus, Bacille Calmette-Guerin (BCG), liposomes, alum, Freund's complete or incomplete adjuvant, detoxified endotoxins, mineral oils, surface active substances such as lipolecithin, pluronic polyols, polyanions, peptides, and oil or hydrocarbon emulsions. Adjuvants, such as aluminum hydroxide or aluminum phosphate, can be added to increase the ability of the vaccine to trigger, enhance, or prolong an immune response. Additional materials, such as cytokines, chemokines, and bacterial nucleic acid sequences, like CpG, a toll-like receptor (TLR) 9 agonist as well as additional agonists for TLR 2, TLR 4, TLR 5, TLR 7, TLR 8, TLR9, including lipoprotein, LPS, monophosphoryllipid A, lipoteichoic acid, imiquimod, resiquimod, and in addition retinoic acid-inducible gene I (RIG-I) agonists such as poly TC, used separately or in combination with the described compositions are also potential adjuvants. Such antigens and anjuvants may be provided as a pharmaceutically acceptable salt, where appropriate.

### Administration, Pharmaceutical Formulations, Dosage Forms

The compounds of the invention can be administered to patients (e.g., animals and humans) in need of such treatment in appropriate dosages that will provide prophylactic and/or therapeutic efficacy. The dose required for use in the treatment or prevention of any particular disease or disorder will typically vary from patient to patient depending on, for example, particular compound or composition selected, the route of administration, the nature of the condition being treated, the age and condition of the patient, concurrent medication or special diets then being followed by the patient, and other factors. The appropriate dosage can be determined by the treating physician.

A compound of this invention can be administered orally, subcutaneously, topically, parenterally, intratumorally or by inhalation spray or rectally in dosage unit formulations containing conventional non-toxic pharmaceutically acceptable carriers, adjuvants and vehicles. Parenteral administration can involve subcutaneous injections, intravenous or intramuscular injections or infusion techniques.

Treatment duration can be as long as deemed necessary by a treating physician. The compositions can be administered one to four or more times per day. A treatment period can terminate when a desired result, for example a particular therapeutic effect, is achieved. Or a treatment period can be continued indefinitely.

Pharmaceutical compositions that include the compounds of the invention are also provided. For example, the present invention provides a pharmaceutical composition comprising a compound of the invention, or a pharmaceutically acceptable salt thereof, and at least one pharmaceutically acceptable carrier.

In some embodiments, the pharmaceutical compositions can be prepared as solid dosage forms for oral administration (e.g., capsules, tablets, pills, dragees, powders, granules and the like). A tablet can be prepared by compression or molding. Compressed tablets can include one or more binders, lubricants, glidants, inert diluents, preservatives, disintegrants, or dispersing agents. Tablets and other solid dosage forms, such as capsules, pills and granules, can include coatings, such as enteric coatings.

Compositions for inhalation or insufflation include solutions and suspensions in pharmaceutically acceptable aqueous or organic solvents, or mixtures thereof, and powders. Liquid dosage forms for oral administration can include, for example, pharmaceutically acceptable emulsions, microemulsions, solutions, suspensions, syrups and elixirs. Suspensions can include one or more suspending agents

Dosage forms for transdermal administration of a subject composition include powders, sprays, ointments, pastes, creams, lotions, gels, solutions, patches and inhalants.

Compositions and compounds of the present invention can be administered by aerosol which can be administered, for example, by a sonic nebulizer.

Pharmaceutical compositions of this invention suitable for parenteral administration include a compound of the invention together with one or more pharmaceutically acceptable sterile isotonic aqueous or non-aqueous solutions, dispersions, suspensions or emulsions. Alternatively, the composition can be in the form of a sterile powder which can be reconstituted into a sterile injectable solutions or dispersion just prior to use.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

¹H NMR Spectra were acquired on one or more of three instruments: (1) Agilent UnityInova 400 MHz spectrometer equipped with a 5 mm Automation Triple Broadband (ATB) probe (the ATB probe was simultaneously tuned to ¹H, ¹⁹F and ¹³C); (2) Agilent UnityInova 400 MHz spectrometer; or (3) Varian Mercury Plus 400 MHz spectrometer. Several NMR probes were used with the 400 MHz NMR spectrometer, including both 3 mm and 5 mm ¹H, ¹⁹F and ¹³C probes and a 3 mm X¹H¹⁹F NMR probe (usually X is tuned to ¹³C). For typical ¹H NMR spectra, the pulse angle was 45 degrees, 8 scans were summed and the spectral width was 16 ppm (−2 ppm to 14 ppm). Typically, a total of about 32768 complex points were collected during the 5.1 second acquisition time, and the recycle delay was set to 1 second. Spectra were collected at 25° C. ¹H NMR Spectra were typically processed with 0.3 Hz line broadening and zero-filling to about 131072 points prior to Fourier transformation. Chemical shifts were expressed in ppm relative to tetramethylsilane. The following abbreviations are used herein: br=broad signal, s=singlet, d=doublet, dd=double doublet, ddd=double double doublet, dt=double triplet, t=triplet, td=triple doublet, tt=triple triplet q=quartet, m=multiplet.

Liquid chromatography-mass spectrometry (LC/MS) experiments to determine retention times and associated mass ions were performed using one or more of the following Methods A, B, and C:An API 150EX mass spectrometer linked to a Shimadzu LC-10AT LC system with a diode array detector was used. The spectrometer had an electrospray source operating in positive and negative ion mode. LC was carried out using an Agilent ZORBAX XDB 50×2.1 mm C18 column and a 0.5 mL/minute flow rate. Solvent A: 95% water, 5% acetonitrile containing 0.01% formic acid; Solvent B: acetonitrile. The gradient was shown as below. 0-0.5 min: 2% solvent (B); 0.5-2.5 min: 2% solvent B to 95% solvent (B); 2.5-4.0 min: 95% solvent (B); 4.0-4.2 min: 95% solvent (B) to 2% solvent B; 4.2-6.0 min: 2% solvent (B).Compounds which required column chromatography were purified manually or fully automatically using either a Biotage SP1™ Flash Purification system with Touch Logic Control™ or a Combiflash Companion® with pre-packed silica gel Isolute® SPE cartridge, Biotage SNAP cartridge or Redisep® Rf cartridge respectively.

### Preparation of Benzobisthiazole Intermediates

The following amines shown in Table I were used in preparing the compounds of the invention. They are either commercially available or can be prepared by known synthetic procedures. CAS registry numbers are provided for each.

TABLE 1
Commercial benzobisthiazoles.

| Int. | | | |
|---|---|---|---|
| No. | Structure | Name | CAS No. |
| 1 | 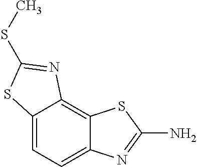 | 11-(methylsulfanyl)-3,10- dithia-5,12-diazatricyclo [7.3.0.0^{2,6}]dodeca-1(9), 2(6),4,7,11-pentaen-4-amine | 1421494-73-6 |
| 2 | 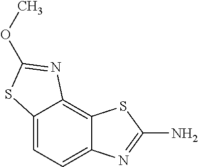 | 11-methoxy-3,10-dithia-5,12- diazatricyclo[7.3.0.0^{2,6}]dodeca- 1(9),2(6),4,7,11-pentaen-4- amine | 1421494-32-7 |
| 3 | 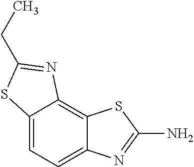 | 11-ethyl-3,10-dithia-5,12- diazatricyclo[7.3.0.0^{2,6}]dodeca- 1(9),2(6),4,7,11-pentaen-4- amine | 1421458-03-8 |
| 4 | 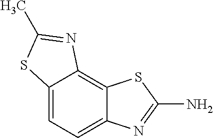 | 2-amino-7-methyl-(7CI,8CI)- Benzo[1,2-d:3,4-d′]bisthiazole | 10023-31-1 |

### Intermediate 5: 8H-Imidazo[4,5-g]benzothiazol-2-amine

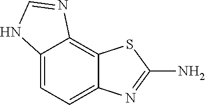 [SMILES: Nc1nc2ccc3[nH]cnc3c2s1]

Step 1: To a solution of 2,4-dinitroaniline (500 mg, 2.7 mmol) in EtOH (5 mL) was added Pd/C (25 mg) and hydrazine hydrate (860 mg, 13.7 mmol) in turn at RT. The reaction mixture was stirred at room temperature for 1 h and filtered through Celite. The filtrate was treated with a saturated aq. solution of NaHCO₃ and extracted with ethyl acetate. The organic phase was dried over Na₂SO₄ and concentrated in vacuo to provide a residue, which was purified by silica gel column (Hex/EA from 20:1 to 2:1) to provide 4-nitrobenzene-1,2-diamine (300 mg, 72%) as a brown solid. LC/MS (ES⁺) calcd. for C₆H₇N₃O₂: 153.05; found: 154.1 [M+H]. ¹H NMR (400 MHz, DMSO-d₆): δ 7.43-7.38 (m, 2H), 6.53 (d, J=8.57 Hz, 1H), 6.03 (s, 2H), 5.05 (s, 2H).

Step 2: A solution of 4-nitrobenzene-1,2-diamine (3.0 g, 20 mmol) in triethyl orthoformate (40 mL) was heated at 100° C. for 12 h. The solution was removed in vacuo to provide a residue, which was purified by silica gel column (DCM/MeOH from 100:1 to 20:1) to afford 6-nitro-1H-benzo[d]imidazole (1.15 g, 36%) as a yellow solid. LC/MS (ES⁺) calcd. for C₇H₅N₃O₂: 163.04; found: 164.1 [M+H].

Step 3: A mixture of 6-nitro-1H-benzo[d]imidazole (915 mg, 5.6 mmol) and Pd/C (190 mg) in MeOH (29 mL) was reacted under a hydrogen balloon. The reaction mixture was stirred at room temperature for 12 h and filtered through Celite. The filtrate was concentrated in vacuo to provide a crude product. The crude product was stirred in MBTE (5 mL) and filtered to afford 8H-imidazo[4′,5′:3,4]benzo[1,2-d] thiazol-2-amine (640 mg, 86%) as a yellow solid. LC/MS (ES⁺) calcd. for C₇H₇N₃: 133.06; found: 134.2 [M+H]. ¹H NMR (400 MHz, DMSO-d₆): δ 11.80 (s, 1H), 7.83 (s, 1H), 7.27 (d, J=8.32 Hz, 1H), 6.62 (s, 1H), 6.49 (d, J=8.32 Hz, 1H), 4.85 (s, 2H).

Step 4: To a solution of 1H-benzo[d]imidazol-6-amine (130 mg, 1.0 mmol) in AcOH (5.2 mL) was added NH₄SCN (340 mg, 4 mmol) at 15° C. The resulting mixture was stirred at 15° C. for 30 min. Then Br₂ (318 mg, 2.0 mmol) was added at 15° C. under N₂, and the resulting mixture was stirred at 15° C. for another 1 h. The reaction mixture was filtered to provide a cake, which was purified by silica gel column (DCM/MeOH from 50:1 to 20:1) to afford the desired product (60 g, 33%) as a white foam. LC/MS (ES⁺) calcd. for C₈H₆N₄S: 190.03; found: 191.1 [M+H]. ¹H NMR (400 MHz, DMSO-d₆): δ 13.06-12.43 (br, 1H), 8.19 (s, 1H), 7.41 (d, J=8.53 Hz, 1H), 7.27 (d, J=8.54 Hz, 1H), 7.26-7.22 (br, 2H),

### Intermediate 5′: 5-thia-3,10,12-triazatricyclo[7.3.0.0{2,6}]dodeca-1,3,6,8,10-pentaen-11-amine

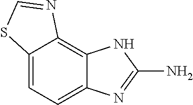 [SMILES: Nc1nc2ccc3scnc3c2[nH]1]

To a solution of 4,5-benzothiazolediamine (CAS No. 1154534-78-7, 5 g, 30 m mol) in 35 mL of aqueous methanol (50% v/v) was added cyanogen bromide (3.1 g, 30 m mol). The reaction was stirred for 24 h, then the solvent was removed in vacuo. The pH was adjusted to 8.5 with aqueous ammonia to precipitate the title compounds (85%) as a white solid. LC/MS (ES⁺) calcd for C₈H₆N4S: 190.2; found: 191.1 [M+H]. ¹H NMR (400 MHz, DMSO-d6): δ 9.15 (s, 3H), 7.66 (d, J=7.50 Hz, 4H), 7.39 (d, J=7.50 Hz, 4H), 6.99 (s, 6H).

### Intermediate 6: Benzo[1,2-d:3,4-d′]bis(thiazole)-2-amine (“3,10-dithia-5,12-diazatricyclo[7.3.0.0{2,6}]dodeca-1,4,6,8,11-pentaen-11-amine”)

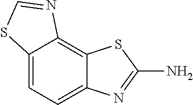 [SMILES: Nc1nc2ccc3scnc3c2s1]

Step 1: A solution of 2-chloro-5-nitroaniline (CAS No. 6283-25-6, 5 g, 0.029 mol) in formic acid (250 mL) was heated at 100-105° C. for 16-18 h. After the reaction was complete (greater than 99% as judged by HPLC), the mixture was cooled and then poured into cold water (800 mL) in a beaker with stirring. Stirring continued for 20-30 min. This afforded a yellow precipitate. The solid was isolated by filtration through a coarse sintered filter glass funnel. The cake was washed with cold water (200 mL) and air dried in a glass tray for 12 h. Subsequent drying at RT under vacuum (5-10 mm of Hg, vacuum oven) afforded N-(2-chloro-5-nitrophenyl)formamide as yellow solid (5.7 g, yield 96%, HPLC 98.2%). LC/MS (ES⁺) calcd for C₇H₅N₂O₃Cl: 200.6; found: 201.6 [M+H]. ¹H NMR (400 MHz, DMSO-d6): δ 10.32 (s, 1H), 9.09 (d, J=3.6 Hz, 1H), 8.44 (s, 1H), 7.96 (d, J=9 Hz, 1H), 7.81 (dd, J=39, 2.8 Hz, 1H) ¹H NMR (400 MHz, CDCl₃): δ 9.20 (s, 1H), 9.00 (d, J=2.0 Hz, 1H), 8.34 (dd, J=8.8, 2.0 Hz, 1H), 8.11 (d, J=8.8 Hz, 1H).

Step 2: To a suspension of N-(2-chloro-5-nitrophenyl)formamide (5.6 g, 0.02 mol) in EtOH (800 mL) was heated at 85-90° C. (gentle reflux). Na₂S-9H₂O (8 g, 0.03 mol, 1.2 eq.) was added in five installments over 40-60 min. After the addition, gentle refluxing was continued for 0.5-1 h. Progress of the reaction was monitored by HPLC (conversion >99%, product ˜75%). The resulting mixture was cooled down to RT and poured in ice-water (1.2 L) with stirring in a large bucket. Then the mixture was brought to a pH of about 1 using concentrated HCl with stirring for 40-60 min. The solid was isolated by filtration through a coarse sintered filter glass funnel. The cake was washed with cold water (200 mL) and air dried in a glass tray for 12 h. Subsequent drying under vacuum at RT (5-10 mm of Hg, vacuum oven) afforded 5-nitro-1,3-benzothiazole as yellow solid (4 g, yield 78%, HPLC 87%). LC/MS (ES⁺) calcd. for C₇H₄N₂O₂S: 180.0; found: 181.0 [M+H]. ¹H NMR (400 MHz, CDCl₃): δ 9.21 (s, 1H), 8.95 (d, J=2.0 Hz, 1H), 8.35 (dd, J=8.8, 2.0 Hz, 1H), 8.12 (d, J=8.8 Hz, 1H).

Step 3: To a suspension of stirring 5-nitrobenzo[d]thiazole (3. g, 0.02 mol) and iron powder (3.55 g, 0.06 mol) in ethanol (50 mL) was added AcOH (5 mL). The resulting mixture was heated to 80-85° C. and stirred for 3.5-4 h. Progress of the reaction was monitored by HPLC. The reaction mixture was diluted with additional 100 mL of EtOH, cooled to 55-60° C. and filtered through Celite using M-type sintered filter glass funnel. The cake was washed with hot ethanol (200 mL). The combined filtrate was concentrated to 5-10 mL and diluted with IPA (30 mL). The mixture was then adjusted to pH of about 9-10 using 30% aq. NaOH with stirring. The layer of IPA was decanted off and the extraction with IPA was repeated two more times (2×20 mL). Combined IPA fractions were concentrated using rotary evaporation under vacuum to obtain crude product.

The crude solid was treated with DCM/hexanes mixture at 55-60° C. for 1-2 h. After cooling to RT the slurry was filtered through a sintered filter glass funnel (M-type) to obtain the desired product. The solid was dried at 20-25° C./5-10 mmHg for 24 h to afford 1.8 g (57%) as a yellow solid (HPLC 97.6%). LC/MS (ES+) calcd. for C7H6N2S: 150.0; found: 151.1 [M+H]. ¹H NMR (400 MHz, DMSO-d6): δ 9.14 (s, 1H), 7.70 (d, J=8 Hz, 1H), 7.17 (s, 1H), 6.79 (d, J=8 Hz, 1H), 5.28 (brs, 2H).

Step 4: To a solution of benzo [d]thiazol-5-amine (1.4 g, 6.6 mmol) in AcOH (20 mL) was added NH₄SCN (2.1 g, 0.03 mol) at 18-20° C. The resulting mixture was stirred at 18-20° C. for 30 min. To this mixture was added Br₂ (0.7 mL, 0.01 mol) drop-wise from an addition funnel at 18-20° C. under N₂. This temperature was maintained at 18-20° C. during addition. The resulting mixture was stirred at 18-20° C. for another 1.5-2 h. Reaction progress was monitored by HPLC. The reaction mixture was then concentrated to minimum volume of AcOH (˜2 mL), diluted with ice-water (20 mL) and treated with 50% aq. NaOH to obtain pH of about 9-10 with stirring. The resulting solids were filtered through an M sintered filter glass funnel, washed with water (10-15 mL), and air dried for 12 h in a tray. This crude solid was treated with a DCM-MeOH mixture (1:1, 15 mL) at 55-60° C. for 1-1.5 h. The insoluble material was filtered through sintered filter glass funnel (M-type) and washed with a DCM-MeOH mixture (1:1, 10 mL). The combined mother liquor was concentrated and dried under vacuum at RT (5-10 mm of Hg, vacuum oven) to obtain the title produce as yellow solid (129 g, yield 92%, HPLC 93.7%). LC/MS (ESI) calcd. for C₈H₅N₃S₂: 207.0; found: 208.0[M+H]. ¹H NMR (400 MHz, DMSO-d6): δ:9.39 (s, 1H), 7.93 (d, J=8.4 Hz, 1H), 7.59 (s, 2H), 7.49 (d, J=8.4 Hz, 1H).

### Intermediate 7: Benzyl benzofuro[7,6-d]thiazol-7-yl-carbamate

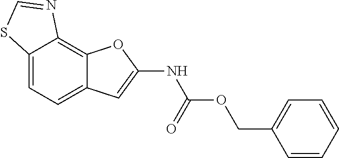 [SMILES: O=C(Nc1cc2ccc3scnc3c2o1)OCc1ccccc1]

Step 1: To a mixture of 3-bromo-2-fluoroaniline (19.0 g, 0.10 mol) in CH₃CN (300 mL) was added benzoyl isothiocyanate (17.1 g, 0.105 mol) at RT. The resulting mixture was stirred at RT for 30 min. The reaction mixture was filtered to afford N-((3-bromo-2-fluorophenyl) carbamothioyl)benzamide as a white solid (32 g, 91%). LC/MS (ES⁺) calcd for C₁₄H₁₀BrFN₂OS: 351.97; found: 353.0 [M+H]. ¹H NMR (400 MHz, CDCl₃): δ 12.75 (s, 1H), 9.17 (s, 1H), 8.37 (t, J=7.2 Hz, 1H), 7.92 (d, J=7.6 Hz, 2H), 7.68 (t, J=7.6 Hz, 1H), 7.56 (t, J=7.2 Hz, 1H), 7.47 (t, J=7.2 Hz, 1H), 7.10 (t, J=8.0 Hz, 1H).

Step 2: To a suspension of 3-bromo-2-fluoroaniline (18.0 g, 50.96 mmol) in MeOH (100 mL) was added NaOH (2 N, 127 mL) at RT, and the resulting mixture was refluxed for 1 h. The reaction mixture was concentrated and extracted with EtOAc. The combined organics were washed with brine, dried over Na₂SO₄, and concentrated to afford 1-(3-bromo-2-fluorophenyl)thiourea as a white solid (11.2 g, 97%). LC/MS (ES⁺) calcd for C₇H₆BrFN₂S: 247.94; found: 248.9[M+H]. ¹H NMR (400 MHz, DMSO-d6): δ 9.48 (s, 1H), 8.02 (br, 1H), 7.62 (t, J=7.2 Hz, 1H), 7.51-7.55 (m, 1H), 7.35 (br, 3H), 7.11-7.15 (m, 1H).

Step 3: To a suspension of 3-bromo-2-fluoroaniline (12.0 g, 48.17 mmol) in CHCl₃ (300 mL) was added a solution of Br₂ (7.7 g, 48.17 mmol) in CHCl₃ (10 mL) at 0° C. The resulting mixture was refluxed for 3 days. The reaction mixture was concentrated. The residue was diluted with saturated aqueous NaHCO₃ solution and extracted with ethyl acetate. The combined organics were washed with brine, dried over Na₂SO₄, and concentrated. The crude product was purified through column chromatography (hexane/EtOAc, 4/1 v/v) to afford 5-bromo-4-fluorobenzo[d]thiazol-2-amine as a light yellow solid (3.5 g, 29%). LC/MS (ES⁺) calcd for C₇H₄BrFN₂S: 245.93; found: 246.8 [M+H]. ¹H NMR (400 MHz, DMSO-d6): δ 7.92 (s, 2H), 7.47 (d, J=8.4 Hz, 1H), 7.22-7.26 (m, 1H).

Step 4: To a solution of 5-bromo-4-fluorobenzo[d]thiazol-2-amine (3.0 g, 12.14 mmol) in THF (20 mL) was added isoamyl nitrite (3.1 g, 26.71 mmol) at RT. The resulting mixture was refluxed for 3 h. The reaction mixture was concentrated and purified through column chromatography (hexane/EtOAc=20/1) to afford 5-bromo-4-fluorobenzo[d]thiazole as a light yellow solid (2.4 g, 85%). LC/MS (ES⁺) calcd for C₇H₃BrFNS: 232.91; found: 233.8 [M+H]. ¹H NMR (400 MHz, DMSO-d6): δ 9.49 (s, 1H), 8.01 (d, J=8.8 Hz, 1H), 7.76-7.80 (m, 1H).

Step 5: A mixture of 5-bromo-4-fluorobenzo[d]thiazole (2.3 g, 9.91 mmol), Zn(CN)₂ (931 mg, 7.93 mmol), Zn (162 mg, 2.48 mmol), Pd₂(dba)₃ (454 mg, 0.50 mmol), and dppf (439 mg, 0.79 mmol) in NMP (20 mL) was stirred at 110° C. for 5 h. The reaction mixture was diluted with water and extracted with EtOAc. The combined organics were washed with brine, dried over Na₂SO₄, and concentrated. The crude product was purified through column chromatography (hexane/EtOAc, 10/1) to afford 4-fluorobenzo[d]thiazole-5-carbonitrile as a light yellow solid (1.3 g, 74%). LC/MS (ES⁺) calcd for C₈H₃FN₂S: 178.00; found: 179.0 [M+H]. ¹HNMR (400 MHz, CDCl₃): δ 9.14 (s, 1H), 7.86 (d, J=8.4 Hz, 1H), 7.63-7.66 (m, 1H).

Step 6: To a solution of 4-fluorobenzo[d]thiazole-5-carbonitrile (1.0 g, 5.61 mmol) in pyridine (12 ml)-water (6 ml)-acetic acid (6 ml) was added sodium hypophosphite (2.41 g, 28.06 mmol) and Raney-Ni (85% in water) (3.2 g, 56.10 mmol) at RT, and the resulting mixture was heated at 50° C. for 2 h. After cooling to RT, the reaction mixture was diluted with water, and extracted with ethyl acetate. The combined organics were washed with 1 N hydrochloric acid and brine, dried over Na₂SO₄, and concentrated. The crude product was purified by column chromatography (hexane/ethyl acetate, 10/1 v/v) to afford 4-fluorobenzo[d]thiazole-5-carbaldehyde as a white solid (360 mg, 34%). LC/MS (ES⁺) calcd for C₈H₄FNCOS: 181.00; found: 182.0[M+H]. ¹H NMR (400 MHz, CDCl₃): δ 10.58 (s, 1H), 9.11 (s, 1H), 7.96 (dd, J=8.4 Hz, 5.6 Hz, 1H), 7.84 (d, J=8.8 Hz, 1H).

Step 7: To a solution of ethyl 2-hydroxyacetate (207 mg, 1.99 mmol) in DMF (4 mL) was added NaH (159 mg, 3.98 mmol, 60%) under N₂ at 0° C. The resulting mixture was stirred at 0° C. for 30 min followed by the addition of a solution of 4-fluorobenzo[d]thiazole-5-carbaldehyde (360 mg, 1.99 mmol) in DMF (4 mL). The resulting mixture was stirred at RT for 1 h. The reaction mixture was quenched with water. 2 N aqueous NaOH solution (4 mL) was added, and the resulting mixture was stirred for 1 hour. The reaction mixture was adjusted to pH 1-2 with 1 N hydrochloric acid and extracted with ethyl acetate. The precipitate formed was filtered and the cake was dried to afford benzofuro[7,6-d]thiazole-7-carboxylic acid as a light yellow solid (150 mg, 34%). LC/MS (ES⁺) calcd for C₁₀H₅NO₃S: 219.00; found: 220.0 [M+H]. ¹H NMR (400 MHz, DMSO-d6): δ 13.62 (s, 1H), 9.55 (s, 1H), 8.13 (d, J=8.4 Hz, 1E1), 7.88 (d, J=8.4 Hz, 1H), 7.86 (s, 1H).

Step 8: A solution of benzofuro[7,6-d]thiazole-7-carboxylic acid (150 mg, 0.68 mmol), DPPA (226 mg, 0.82 mmol), and DIPEA (106 mg, 0.82 mmol) in toluene (4 mL) was heated at 85° C. for 30 min. Phenylmethanol (110 mg, 1.02 mmol) was added, and the resulting mixture was stirred at 85° C. for 12 h. The reaction mixture was concentrated. The residue was diluted with ethyl acetate, washed with brine, and dried over Na₂SO₄, The organic layer was concentrated and purified by column chromatography (hexane/ethyl acetate, 5/1 v/v) to afford benzyl benzofuro[7,6-d]thiazol-7-ylcarbamate as a white solid (190 mg, 86%). LC/MS (ES⁺) calcd for C₁₇H₁₂N₂O₃S: 324.06; found: 325.1 [M+H]. ¹H NMR (400 MHz, CDCl₃): δ 9.01 (s, 1H), 7.76 (d, J=8.4 Hz, 1H), 7.72 (br, 1H), 7.58 (d, J=8.0 Hz, 1H), 7.38-7.45 (m, 5H), 6.73 (br, 1H), 5.29 (s, 2H).

### Intermediate 8: 3,10-dithia-5-azatricyclo[7.3.0.0{2,6}]dodeca-1,4,6,8,11-pentaen-11-amine

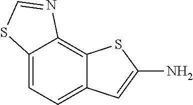 [SMILES: Nc1cc2ccc3scnc3c2s1]

Step 1: To a suspension of 2-chloro-3-nitrobenzaldehyde (CAS No. 58755-57-0, 9.4 g, 50.6 mmol) and K₂CO₃ (7.7 g, 55.7 mmol) in DMF (80 mL) was added dropwise methyl 2-mercaptoacetate (5.48 g, 51.6 mmol) at 0-5° C. The resulting mixture was stirred at RT for 12 h. The reaction mixture was diluted with water. The precipitates formed were filtered, washed with water, and dried to afford methyl 7-nitrobenzo[b]thiophene-2-carboxylate as a yellow solid (11.5 g, 95%). LC/MS (ES⁺): m/z calculated for C₁₀H₇NO₄S: 237.0; found: 238.4 [M+H]. ¹H NMR (400 MHz, CDCl₃): δ 8.53 (d, J=8.0 Hz, 1H), 8.23 (d, J=8.0 Hz, 1H), 8.19 (s, 1H), 7.61 (t, J=8.0 Hz, 1H), 3.99 (s, 3H).

Step 2: To a suspension of methyl 7-nitrobenzo[b]thiophene-2-carboxylate (12.0 g, 50.6 mmol) and Fe powder (14.2 g, 253 mmol) in MeOH (150 nil) was added aqueous NH₄Cl (18.9 g, 354 mmol). The resulting mixture was refluxed for 4 h. After the reaction mixture was filtered, the filtrate was concentrated and diluted with water. The precipitates formed were filtered, washed with water, and dried to afford methyl 7-aminobenzo [b]thiophene-2-carboxylate as a yellow solid (9.2 g, 87%). LC/MS (ES⁺): m/z calculated for C₁₀H₉NO₂S: 207.0; found: 208.0 [M+H]. ¹H NMR (400 MHz, CDCl₃): δ 8.05 (s, 1H), 7.38 (d, J=8.0 Hz, 1H), 7.26 (t, J=8.0 Hz, 1H), 6.78 (d, J=7.6 Hz, 1H), 3.95 (s, 3H), 3.93 (br, 2H).

Step 3: To a solution methyl 7-aminobenzo[b]thiophene-2-carboxylate (200 mg, 0.96 mmol) in MeCN (5 ml) was added dropwise benzoyl isothiocyanate (173 mg, 1.06 mmol). The resulting mixture was stirred at RT for 0.5 h. The precipitates formed were filtered, washed with MeCN, and dried to afford methyl 7-(3-benzoylthioureido) benzo[b]thiophene-2-carboxylate as a yellow solid (290 mg, 83%). LC/MS (ES⁺): mlz calculated for C₁₈H₁₄N₂O₃S₂: 370.0; found: 371.1 [M+H]. ¹H NMR (400 MHz, CDCl₃): δ 12.70 (br, 1H), 9.23 (br, 1H), 8.13 (s, 1H), 8.07 (d, J=7.2 Hz, 1H), 7.96 (d, J=7.6 Hz, 2H), 7.87 (d, J=8.0 Hz, 1H), 7.69 (t, J=7.4 Hz, 1H), 7.58 (t, J=7.6 Hz, 2H), 7.51 (t, J=7.8 Hz, 1H), 3.95 (s, 3H).

Step 4: To a suspension methyl 7-(3-benzoylthioureido) benzo[b]thiophene-2-carboxylate (290 mg, 0.78 mmol) in methanol (5 nil) was added NaOH (250 mg, 6.26 mmol). The resulting mixture was refluxed for 2 h. After methanol was removed, 2 M hydrochloric acid was added to the residue to adjust to pH 5-6. The precipitates formed were filtered, washed with water, and dried to afford 7-thioureidobenzo[b]thiophene-2-carboxylic acid as a pale yellow solid (150 mg, 76%). LC/MS (ES⁺): mlz calculated for C₁₀H₈N₂O₂S₂: 252.0; found: 253.0 [M+H]. ¹H NMR (400 MHz, DMSO-d6): δ 13.51 (br, 1H), 9.79 (s, 1H), 8.13 (s, 1H), 7.90 (d, J=6.8 Hz, 1H), 7.50-7.47 (m, 2H), 7.18 (br, 1H).

Step 5: To a suspension 7-thioureidobenzo[b]thiophene-2-carboxylic acid (3.5 g, 13.8 mmol) in AcOH (50 ml) was added dropwise a solution of Br₂ (2.2 g, 13.8 mmol) in AcOH (5 ml). The resulting mixture was stirred at RT for 12 h. The precipitates were filtered, washed with saturated NaHCO₃ solution, and dried to afford 2-aminothieno [3′,2′:5,6] benzo[1,2-d]thiazole-7-carboxylic acid as a pale yellow solid (3 g, 86%). LC/MS (ES⁺): mlz calculated for C₁₀H₆N₂O₂S₂: 250.0; found: 251.0 [M+H]. ¹H NMR (400 MHz, DMSO-d6): δ 8.15 (s, 1H), 7.82 (d, J=8.4 Hz, 1H), 7.71 (d, J=8.4 Hz, 1H).

Step 6: To a suspension of 2-aminothieno [3′,2′:5,6] benzo[1,2-d]thiazole-7-carboxylic acid (3 g, 12 mmol) in THF (50 ml) was added dropwise t-BuONO (2.7 g, 26.3 mmol). The resulting mixture was refluxed for 3 h. After THF was removed, the residue was diluted with water and extracted with DCM/MeOH (v/v 20:1). The combined organic layers were washed with water and brine, dried over Na₂SO₄, and concentrated. The crude product was purified through silica gel column chromatography (DCM/EtOAc=1/1) to afford thieno[3′,2′:5,6]benzo [1,2-d]thiazole-7-carboxylic acid as a yellow solid (2.2 g, 78%). LC/MS (ES⁺): m/z calculated for C₁₀H₅NO₂S₂: 235.0; found: 236.0 [M+H]. ¹H NMR (400 MHz, DMSO-d6): δ 13.64 (br, 1H), 9.58 (s, 1H), 8.31 (s, 1H), 8.24 (d, J=8.4 Hz, 1H), 8.09 (d, J=8.4 Hz, 1H).

Step 7: A solution of thieno[3′,2′:5,6]benzo[1,2-d]thiazole-7-carboxylic acid (200 mg, 0.85 mmol), diphenylphosphoryl azide (350 mg, 1.27 mmol), and triethylamine (130 mg, 1.27 mmol) in t-BuOH (10 ml) was heated at 70° C. for 12 h. The reaction mixture was concentrated and purified through silica gel column chromatography (n-Hex/EtOAc=8/1) to afford tert-butyl thieno[3′,2′:5,6]benzo[1,2-d]thiazol-7-ylcarbamate as a yellow solid (150 mg, 60%). LC/MS (ES⁺): m/z calculated for C₁₄H₁₄N₂O₂S₂: 306.05; found: 307.0 [M+H]. ¹H NMR (400 MHz, CDCl₃): δ 9.05 (s, 1H), 7.84 (d, J=8.4 Hz, 1H), 7.65 (d, J=8.4 Hz, 1H), 7.12 (br, 1H), 6.98 (s, 1H), 1.57 (s, 9H).

Step 8: A mixture of teat-butyl thieno[3′,2′:5,6]benzo[1,2-d]thiazol-7-ylcarbamate (200 mg, 0.65 mmol) in 4.0 M HCl/dioxane was stirred at RT for 3 h. After dioxane was removed, the residue was diluted with water, basified with sat. aqueous NaHCO₃ solution, and extracted with DCM. The combined organic layers were washed with water and brine, dried over Na₂SO₄, and concentrated. The crude product was purified through silica gel column chromatography (DCM/EtOAc=2/1) to afford thieno[3′,2′:5,6]benzo[1,2-d]thiazol-7-amine as a yellow solid (50 mg, 40%). LC/MS (ES⁺): m/z calculated for C₉H₆N₂S₂: 206.0; found: 207.0 [M+H]. ¹H NMR (400 MHz, CDCl₃): δ 9.02 (s, 1H), 7.78 (d, J=8.4 Hz, 1H), 7.52 (d, J=8.4 Hz, 1H) 6.46 (s, 1H), 4.13 (br, 2H).

### Intermediate 8′: 2-methoxythieno[3′,2′:5,6]benzo[1,2-d]thiazol-7-amine

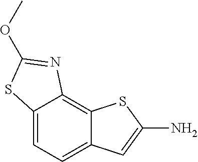

Step 1: To a solution of 2-aminothieno [3′,2′:5,6] benzo [1,2-d] thiazole-7-carboxylic acid (product from Intermediate 8, Step 5; 10.0 g, 40.0 mmol) in DMF (40 mL) was added K₂CO₃ (16.6 g, 120.0 mmol) and MeI (8.5 g, 60 mmol) at room temperature, and the resulting mixture was stirred for 2 h. The reaction was quenched with water (80 mL), and the precipitate was collected through filtration. The filter cake was dissolved in THF, dried over Na₂SO₄, and concentrated in vacuo to afford methyl 2-aminothieno [3′,2′:5,6] benzo [1,2-d] thiazole-7-carboxylate as a yellow solid (7.4 g, 67%). LC/MS (ES⁺) calcd for C₁₁H₈N₂O₂S₂: 264.3; found: 264.9 [M+H].

Step 2: To a suspension of methyl 2-aminothieno [3′,2′:5,6] benzo [1,2-d] thiazole-7-carboxylate (6.5 g, 24.6 mmol) and CuCl₂ (5.0 g, 36.9 mmol) in MeCN was added dropwise a solution of t-BuONO (3.8 g, 36.9 mmol) in MeCN (40 mL) at room temperature, and the resulting mixture was stirred for 2 h. The reaction mixture was quenched with water, extracted with DCM, dried over Na₂SO₄, and concentrated in vacuo to give a residue which was purified through silica gel flash column chromatography (n-Hexane/DCM=10/1˜100% DCM) to afford methyl 2-chlorothieno [3′,2′:5,6] benzo [1,2-d] thiazole-7-carboxylate as a yellow solid (5.4 g, 75%). LC/MS (ES⁺) calcd for C₁₁H₆ClNO₂S₂: 283.8; found: 283.8 [M+H]. ¹H NMR (400 MHz, DMSO-d₆) δ 8.39 (s, 1H), 8.18 (d, J=8.8 Hz, 1H), 8.11 (d, J=8.8 Hz, 1H), 3.93 (s, 3H).

Step 3: To a suspension of methyl 2-chlorothieno [3′,2′:5,6] benzo [1,2-d] thiazole-7-carboxylate (4.0 g, 14.1 mmol) in dry THF (85 mL) was added freshly prepared MeONa solution in MeOH (0.5 M, 85 mL, 42.3 mmol) at room temperature, and the resulting mixture was stirred for 7 h. Water (85 mL) was added to quench the reaction, and the resulting mixture was stirred at room temperature for 12 h. Another portion of water (80 mL) was added, and the resulting mixture was concentrated in vacuo to remove THF and MeOH. The aqueous phase was acidified with hydrochloric acid (1.0 N) to pH=5 at 0° C. and stirred for 1 h. The resulting suspension was filtered and rinsed with water. The filter cake was dried in vacuo to afford 2-methoxythieno [3′,2′:5,6] benzo [1,2-d] thiazole-7-carboxylic acid as a white solid (3.5 g, 90%). LC/MS (ES⁺) calcd for C₁₁H₇NO₃S₂: 265.3; found: 265.9 [M+H]. ¹H NMR (400 MHz, DMSO-d₆) δ 13.54 (br, 1H), 8.21 (s, 1H), 7.98 (d, J=8.4 Hz, 1H), 7.90 (d, J=8.4 Hz, 1H), 4.24 (s, 3H).

Step 4: To a suspension of 2-methoxythieno [3′,2′:5,6] benzo [1,2-d] thiazole-7-carboxylic acid (3.5 g, 13.2 mmol) in toluene (40 mL) was added TEA (2.0 g, 19.8 mmol) and DPPA (5.4 g, 19.8 mmol) at room temperature, and the resulting mixture was stirred at room temperature for 1 h. tert-Butanol (1.37 g, 18.5 mmol) was added, and the resulting mixture was stirred at 100° C. for 12 h. After cooling to room temperature, the reaction mixture was concentrated in vacuo, and the residue was purified through silica gel flash column chromatography (n-Hexane/DCM=5/1 to 100% DCM) to afford tert-butyl (2-methoxythieno [3′,2′:5,6] benzo [1,2-d] thiazol-7-yl) carbamate as a white solid (2.5 g, 57%), LC/MS (ES⁺) calcd for C₁₅H₁₆N₂O₃S₂: 336.4; found: 337.0 [M+H]. ¹H NMR (400 MHz, CDCl₃) δ 7.51 (d, J=8.40 Hz, 1H), 7.42 (d, J=8.40 Hz, 1H), 7.08 (br, 1H), 6.83 (s, 1H), 4.22 (s, 3H), 1.56 (s, 9H).

Step 5: 2-methoxythieno [3′,2′:5,6] benzo [1,2-d] thiazol-7-amine (500 mg, 1,5 mmol) was dissolved in TFA (18 mL) at 0° C., and the resulting mixture was stirred for 1 h. The reaction mixture was poured into a mixture of saturated aq. NaHCO₃ solution (100 mL) and EtOAc (100 mL) at 0° C. with vigorous stirring. The organic phase was washed with brine, dried over Na₂SO₄, and concentrated in vacuo to give a crude product which was triturated with n-hexane to afford the title compound as an off-white solid (290 mg, 83%). LC/MS (ES⁺) calcd for C₁₀H₈N₂OS₂: 236.3; found: 236.8 [M+H]. ¹H NMR (400 MHz, DMSO-d₆) δ 7.57 (d, J=8.40 Hz, 1H), 7.25 (d, J=8.40 Hz, 1H), 6.11 (s, 1H), 6.07 (br, 2H), 4.17 (s, 3H).

### Intermediate 9: 10-oxa-3-thia-5-azatricyclo[7.3.0.0{2,6}]dodeca-1,4,6,8,11-pentaen-11-amine

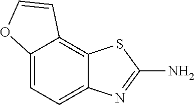 [SMILES: Nc1nc2ccc3occc3c2s1]

Step 1: To a mixture of 3-bromo-2-fluoroaniline (19.0 g, 0.10 mol) in CH₃CN (300 mL) was added benzoyl isothiocyanate (17.1 g, 0.105 mol) at RT. The mixture was stirred at RT for 30 min, and then filtered to afford N-((3-bromo-2-fluorophenyl) carbamothioyl) benzamide as a white solid (32 g, 91%). LC/MS (ES⁺) calcd for C₁₄H₁₀BrFN₂OS: 352.0; found: 353.0 [M+H]. ¹H NMR (400 MHz, CDCl₃): δ 12.75 (s, 1H), 9.17 (s, 1H), 8.37 (t, J=7.2 Hz, 1H), 7.92 (d, J=7.6 Hz, 2H), 7.68 (t, J=7.6 Hz, 1H), 7.56 (t, J=7.2 Hz, 1H), 7.47 (t, J=7.2 Hz, 1H), 7.10 (t, J=8.0 Hz, 1H).

Step 2: To a suspension of N-((3-bromo-2-fluorophenyl) carbamothioyl)benzamide (18.0 g, 50.96 mmol) in MeOH (100 mL) was added 2N aq. NaOH (127 mL) at ambient temperature. The mixture was stirred under reflux for 1 h. The reaction was diluted with EtOAc, washed with brine, dried over Na₂SO₄ and concentrated to afford 1-(3-bromo-2-fluorophenyl) thiourea as a white solid (11.2 g, 97%). LC/MS (ES⁺) calcd for C₇H₆BrFN₂S: 247.9; found: 248.9[M+H]. ¹H NMR (400 MHz, DMSO-d6): δ 9.48 (s, 1H), 8.02 (br, 1H), 7.62 (t, J=7.2 Hz, 1H), 7.55-7.51 (m, 1H), 7.35 (br, 1H), 7.16-7.10 (m, 1H).

Step 3: To a solution of Br₂ (7.7 g, 48.17 mmol) in CHCl₃ (10 mL) was added to a suspension of 1-(3-bromo-2-fluorophenyl)thiourea (12.0 g, 48.17 mmol) in CHCl₃ (300 mL) at 0° C. The mixture was stirred under reflux for 3 days. The reaction mixture was concentrated. The residue was partitioned into saturated aq. NaHCO₃ and extracted with ethyl acetate. The combined organics were washed with brine, dried over Na₂SO₄ and concentrated. The crude product was purified by column chromatography (hexane/EtOAc=4/1) to afford 5-bromo-4-fluorobenzo[d]thiazol-2-amine as a light yellow solid (3.5 g, 29%). LC/MS (ES⁺) calcd for C₇H₄BrFN₂S: 245.9; found: 246.8 [M+H]. ¹H NMR (400 MHz, DMSO-d6): δ 7.92 (s, 2H), 7.47 (d, J=8.4 Hz, 1H), 7.22-7.26 (m, 1H).

Step 4: To a solution of 5-bromo-4-fluorobenzo[d]thiazol-2-amine (3.0 g, 12.14 mmol) in THF (20 mL) was added isoamyl nitrite (3.1 g, 26.71 mmol) at RT. The mixture was stirred under reflux for 3 h. The reaction mixture was concentrated and purified by column chromatography (hexane/EtOAc=20/1) to afford 5-bromo-4-fluorobenzo [d]thiazole as a light yellow solid (2.4 g, 85%). LC/MS (ES⁺) calcd for C₇H₃BrFNS: 232.9; found: 233.8 [M+H]. ¹H NMR (400 MHz, DMSO-d6): δ 9.49 (s, 1H), 8.01 (d, J=8.8 Hz, 1H), 7.76-7.80 (m, 1H).

Step 5: To a mixture of 5-bromo-4-fluorobenzo[d]thiazole (2.3 g, 9.91 mmol), Zn(CN)₂ (931 mg, 7.93 mmol), Zn powder (162 mg, 2.48 mmol), Pd₂(dba)₃ (454 mg, 0.50 mmol) and dppf (439 mg, 0.79 mmol) in NMP (20 mL) was stirred at 110° C. for 5 hours. The reaction mixture was diluted with water and extracted with EtOAc. The combined organics were washed with brine, dried over Na₂SO₄ and concentrated. The crude product was purified by column chromatography (hexane/EtOAc=10/1) to afford 4-fluorobenzo[d]thiazole-5-carbonitrile as a light yellow solid (1.3 g, 74%). LC/MS (ES⁺) calcd for C₈H₃FN₂S: 178.0; found: 179.0[M+H]. ¹H NMR (400 MHz, CDCl₃): δ 9.14 (s, 1H), 7.86 (d, J=8.4 Hz, 1H), 7.63-7.66 (m, 1H).

Step 6: To a solution of 4-fluorobenzo[d]thiazole-5-carbonitrile (1.0 g, 5.61 mmol) in pyridine/H₂O/HOAc (2/1/1, 24 mL) was added sodium hypophosphite (2.41 g, 28.06 mmol) and Raney-Ni (85% in water) (3.2 g, 56.10 mmol) at RT. The mixture was heated at 50° C. for 2 h.

After cooling to RT, the reaction mixture was diluted with water and extracted with ethyl acetate. The combined organics were washed with 1 N HCl, brine, dried over Na₂SO₄ and concentrated. The crude product was purified by column chromatography (hexane/ethyl acetate=10/1) to afford 4-fluorobenzo[d]thiazole-5-carbaldehyde as a white solid (360 mg, 34%). LC/MS (ES⁺) calcd for C₈H₄FNCOS: 181.0; found: 182.0 [M+H]. ¹H NMR (400 MHz, CDCl₃): δ 10.58 (s, 1H), 9.11 (s, 1H), 7.96 (dd, J=8.4 Hz, 5.6 Hz, 1H), 7.84 (d, J=8.8 Hz, 1H).

Step 7: To a solution of ethyl 2-hydroxyacetate (207 mg, 1.99 mmol) in DMF (4 mL) was added NaH (159 mg, 3.98 mmol, 60%) at 0° C. under N₂. The mixture was stirred at 0° C. for 30 min. A solution of 4-fluorobenzo[d]thiazole-5-carbaldehyde (360 mg, 1.99 mmol) in DMF (4 mL) was added. The mixture was stirred at RT for 1 h. The reaction was quenched with water, and 2 N aq. NaOH (4 mL) was added and stirred for 1 h. The mixture was acidified with 1 N aq. HCl. The resulting suspension was filtered and the cake was dried to provide benzofuro [7,6-d]thiazole-7-carboxylic acid as a light yellow solid (150 mg, 34%). LC/MS (ES⁺) calcd for C₁₀H₅NO₃S: 219.0; found: 220.0 [M+H]. ¹H NMR (400 MHz, DMSO-d6): δ 13.62 (s, 1H), 9.55 (s, 1H), 8.13 (d, J=8.4 Hz, 1H), 7.88 (d, J=8.4 Hz, 1H), 7.86 (s, 1H).

Step 8: A solution of benzofuro[7,6-d]thiazole-7-carboxylic acid (150 mg, 0.68 mmol), DPPA (226 mg, 0.82 mmol) and DIPEA (106 mg, 0.82 mmol) in toluene (4 mL) was stirred at 85° C. for 30 min. BnOH (110 mg, 1.02 mmol) was added, and then the mixture was stirred at 85° C. for 12 h. The mixture was diluted with ethyl acetate, washed with brine, and dried over Na₂SO₄. The organic phase was concentrated and purified by column chromatography (hexane/ethyl acetate=5/1) to afford benzyl benzofuro[7,6-d]thiazol-7-ylcarbamate as a white solid (190 mg, 86%). LC/MS (ES⁺) calcd for C₁₇H₁₂N₂O₃S: 324.1; found: 325.1[M+H]. ¹H NMR (400 MHz, CDCl₃): δ 9.01 (s, 1H), 7.76 (d, J=8.4 Hz, 1H), 7.72 (br, 1H), 7.58 (d, J=8.0 Hz, 1H), 7.38-7.45 (m, 5H), 6.73 (br, 1H), 5.29 (s, 2H).

### Intermediate 10: 5,12-dithia-3-azatricyclo[7.3.0.0{2,6}]dodeca-1,3,6,8,10-pentaen-4-amine

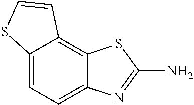 [SMILES: Nc1nc2ccc3sccc3c2s1]

To a solution of benzo[b]thiophen-5-amine (CAS No. 20532-28-9, 2.0 g, 13.4 mmol) in acetic acid (50 mL) was added NH₄SCN (3.0 g, 40.2 mmol) and the mixture was stirred at RT for 1 h. A solution of Br₂ (1 ml, 19.6 mmol) in acetic acid (10 mL) was added dropwise to the above-mentioned mixture at RT. The reaction mixture was stirred for 12 h at RT. The formed precipitates were filtered, washed with water and suspended in sat. aqueous NaHCO₃ again. The solid was collected by filtration and dried to afford the title compound as a green solid (2.4 g, 87%). LC/MS (ES⁺): m/z calculated for C₉H₆N₂S₂: 206.0; found: 207.3 [M+H]. ¹H NMR (400 MHz, DMSO-d6): δ 7.82 (t, J=6.6 Hz, 2H), 7.45 (br, 2H), 7.40 (t, J=7.2 Hz, 2H).

### Intermediate 11: 12-oxa-5-thia-3-azatricyclo[7.3.0.0{2,6}]dodeca-1,3,6,8,10-pentaen-4-amine

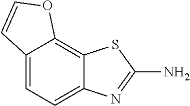 [SMILES: Nc1nc2ccc3ccoc3c2s1]

To a solution of benzofuran-5-amine (CAS No. 58546-89-7, 200 mg, 1.5 mmol) in acetic acid (8 mL) was added ammonium thiocyanate (456 mg, 6.0 mmol) at RT under nitrogen atmosphere. After stirring for 10 min, bromine (480 mg, 3.0 mmol) was added dropwise at about 10° C. The resulting mixture was slowly warmed to RT and stirred for 12 h. The precipitate was collected by filtration to afford the title compound as pale brown solid (200 mg, 70%). LC/MS (ES⁺) calcd for C₉H₆N₂OS: 190.0; found: 193.0 [M+3]. ¹H NMR (400 MHz, DMSO-d6): δ 8.01 (d, J=2.0 Hz, 1H), 7.46 (d, J=8.4 Hz, 1H), 7.36 (s, 2H), 7.32 (d, J=8.4 Hz, 1H), 6.99 (d, J=2.0 Hz, 1H).

### Intermediate 12: 3-oxa-10-thia-5,12-diazatricyclo[7.3.0.0{2,6}]dodeca-1,4,6,8,11-pentaen-11-amine

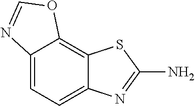 [SMILES: Nc1nc2ccc3ncoc3c2s1]

Step 1: A solution of 2-amino-4-nitrophenol (5.0 g, 32 mmol) in trimethoxymethane (60 mL) was stirred at reflux for 12 h. The reaction mixture was poured into ice water and extracted with EtOAc. The combined organic layers were washed with water and brine, dried over Na₂SO₄, and concentrated under reduced pressure. The concentrate was purified by column chromatography (n-Hex/EtOAc=8/1) to afford 5-nitrobenzo[d]oxazole as an orange solid (4.0 g, 75%). LC/MS (ES⁺): m/z calculated for C₇H₄N₂O₃: 164.0; found: 165.1 [M+H]. ¹H NMR (400 MHz, CDCl₃): δ 8.71 (d, J=1.6 Hz, 1H), 8.39-8.36 (dd, J=9.0 Hz, 1.8 Hz, 1H), 8.27 (s, 1H), 7.73 (d, J=8.8 Hz, 1H).

Step 2: A mixture of 5-nitrobenzo[d]oxazole (13.0 g, 79 mmol) and 10% Pd/C (1.3 g) in methanol (200 mL) was stirred at RT for 12 h, under hydrogen atmosphere. The reaction mixture was filtered, and the filtrate was concentrated under reduced pressure. The concentrate was purified by column chromatography (hexane/EtOAc=2/1) to afford benzo[d]oxazol-5-amine as a brown solid (7.0 g, 66%). LC/MS (ES⁺): m/z calculated for C₇H₆N₂O: 134.1; found: 135.1 [M+H]. ¹H NMR (400 MHz, CDCl₃): δ 7.99 (s, 1H), 7.35 (d, J=8.4 Hz, 1H), 7.05 (d, J=2.4 Hz, 1H), 6.75-6.72 (dd, J=8.6 Hz, 2.2 Hz, 1H), 3.72 (br, 2H).

Step 3: To a solution of benzo[d]oxazol-5-amine (7.0 g, 52 mmol) in acetic acid (120 mL) was added NH₄SCN (11.9 g, 156 mmol) and the mixture was stirred at RT for 1 h. A solution of Br₂ (2.9 ml, 57 mmol) in acetic acid (30 mL) was added to the mixture above by dropwise at RT. The resulting mixture was stirred at RT for 12 h. The resulting suspension was filtered, and the filtrate was concentrated. The concentrate was triturated with saturated aq. NaHCO₃ and extracted with EtOAc. The combined organic layers were washed with water, brine, dried over Na₂SO₄, and concentrated under reduced pressure. The concentrate was purified by column chromatography (hexane/EtOAc 2/1) to afford the title compound as a light yellow solid (150 mg, 1.5%). LC/MS (ES⁺): m/z calculated for C₈H₅N₃OS: 191.0; found: 192.0 [M+H]. ¹H NMR (400 MHz, DMSO-d6): δ 8.77 (s, 1H), 7.63 (d, J=8.8 Hz, 1H), 7.54 (br, 2H), 7.44 (d, J=8.4 Hz, 1H).

### Intermediate 13: 10-oxa-3-thia-5,12-diazatricyclo[7.3.0.0{2,6}]dodeca-1,4,6,8,11-pentaen-11-amine

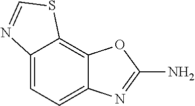 [SMILES: Nc1nc2ccc3ncsc3c2o1]

Step 1: To a stirred mixture of 7-nitrobenzo[d]oxazol-2-amine (CAS No. 95082-02-3, 1.2 g, 6.7 mmol) and DMAP (85 mg, 0.7 mmol) in DCM (15 mL) was added di-tert-butyl dicarbonate (1.75 g, 8 mmol), and the resulting mixture was stirred at RT for 2 h. After this time, the reaction mixture was partitioned between ethyl acetate and water. The organic layer was collected, washed with brine, dried over anhydrous sodium sulfate, and concentrated under reduced pressure to give a crude product which was purified through silica gel flash column chromatography (eluted with 20% ethyl acetate in hexane) to afford tert-butyl (7-nitrobenzo[d]oxazol-2-yl)carbamate as a yellow solid (1.1 g, 61%). LC/MS (ES⁺): m/z calculated for C₁₂H₁₃N₃O₅: 279.2; found: 280.3 [M+H]. ¹H NMR (400 MHz, CDCl₃): δ 1.60 (s, 9H), 7.43 (t, J=8.0 Hz, 1H), 7.95 (d, J=8.0 Hz, 1H), 8.04 (d, J=8.0 Hz, 1H), 8.48 (br, 1H).

Step 2: To a solution of tert-butyl (7-nitrobenzo[d]oxazol-2-yl)carbamate (1.1 g, 279 mmol) in methanol (30 mL) was added palladium on carbon (10%, 100 mg), and the resulting mixture was stirred at RT under hydrogen atmosphere (hydrogen balloon) for 12 h. TLC showed the reaction completed. Pd/C was removed through filtration and rinsed with methanol. The combined filtrate was concentrated under reduced pressure to afford tert-butyl (7-aminobenzo[d]oxazol-2-yl)carbamate as a yellow solid (560 mg, 56%). LC/MS (ES⁺) calcd for C₁₂H₁₅N₃O₃: 249.1; found: 250.1 [M+H]. ¹H NMR (400 MHz, DMSO-d6): δ 1.49 (s, 9H), 5.28 (s, 2H), 6.52 (d, J=8.0 Hz, 1H), 6.71 (d, J=8.0 Hz, 1H), 6.95 (t, J=8.0 Hz, 1H), 11.01 (s, 1H).

Step 3: To a mixture of tert-butyl (7-aminobenzo[d]oxazol-2-yl)carbamate (560 mg, 2.25 mmol) in acetonitrile (20 mL) was added benzoyl isothiocyanate (403 mg, 2.5 mmol), and the resulting mixture was stirred at RT for 2 h. The reaction mixture was then filtered. The filter cake was rinsed with acetonitrile, and the filtrate was dried and concentrated to afford tert-butyl (7-(3-benzoylthioureido)benzo[d] oxazol-2-yl)carbamate as a light yellow solid (820 mg, 88%). LC/MS (ES⁺) calcd for C₂₀H₂₀N₄O₄S: 412.1; found: 413.3 [M+H]. ¹H NMR (400 MHz, DMSO-d6): δ 1.49 (s, 9H), 7.32 (t, J=8.0 Hz, 1H), 7.48 (d, J=8.0 Hz, 1H), 7.55-7.61 (m, 3H), 7.67-7.71 (m, 1H), 8.01 (d, J=7.6 Hz, 2H), 11.35 (s, 1H), 11.88 (s, 1H), 12.57 (s, 1H).

Step 4: A mixture of tert-butyl (7-(3-benzoylthioureido)benzo[d]oxazol-2-yl)carbamate (820 mg, 2.0 mmol) and aqueous sodium hydroxide solution (2 M, 5 mL) in methanol (10 mL) was stirred at 80° C. for 1 h. TLC showed the reaction completed. The reaction mixture was partitioned between ethyl acetate and water. The combined organic layers were washed with brine, dried over anhydrous sodium sulfate, and concentrated under reduced pressure to give a residue which was purified through preparative TLC (eluted with 5% methanol in DCM) to afford tert-butyl (7-thioureidobenzo[d]oxazol-2-yl)carbamate as a light yellow solid (600 mg, 97%). LC/MS (ES⁺) calcd for C₁₃H₁₆N₄O₃S: 308.1; found: 309.1 [M+H].

Step 5: To a stirred mixture of tert-butyl (7-thioureidobenzo[d]oxazol-2-yl)carbamate (300 mg, 0.97 mmol) in chloroform (30 mL) and THF (0.5 mL) was added bromine (155 mg, 0.97 mmol) over 5 min, and the resulting mixture was stirred at RT for 10 min. TLC showed the reaction completed. The reaction mixture was quenched with methanol, and concentrated under reduced pressure to give a residue which was purified through preparative TLC (eluted with 5% methanol in DCM) to afford tert-butyl (7-aminothiazolo [4′,5′:3,4]benzo[1,2-d]oxazol-2-yl)carbamate] as a white solid (150 mg, 50%). LC/MS (ES⁺) calcd for C₁₃H₁₄N₄O₃S: 306.1; found: 307.1 [M+H]. ¹H NMR (400 MHz, DMSO-d6): δ 1.50 (s, 9H), 7.18 (d, J=8.0 Hz, 1H), 7.55 (d, J=8.4 Hz, 1H), 7.73 (s, 2H), 11.17 (s, 1H).

Step 6: A mixture of tert-butyl (7-aminothiazolo[4′,5′:3,4]benzo[1,2-d]oxazol-2-yl)-carbamate (80 mg, 0.26 mmol) and isopentyl nitrite (67 mg, 0.58 mmol) in anhydrous THF (4 mL) was stirred at 80° C. for 2 h. The reaction mixture was then partitioned between ethyl acetate and water. The organic layer was washed with brine, dried over anhydrous sodium sulfate, and concentrated under reduced pressure to give a residue which was purified through preparative TLC (eluted with 5% methanol in DCM) to afford tert-butyl thiazolo[4′,5′:3,4]benzo[1,2-d]oxazol-2-ylcarbamate as a light yellow solid (60 mg, 78%). LC/MS (ES⁺) calcd for C₁₃H₁₃N₃O₃S: 291.1; found: 292.1 [M+H]. ¹H NMR (400 MHz, DMSO-d6): δ 1.53 (s, 9H), 7.72 (d, J=8.8 Hz, 1H), 8.08 (d, J=8.4, 1H), 9.52 (s, 1H), 11.37 (s, 1H).

Step 7: A mixture of tert-butyl thiazolo[4′,5′:3,4]benzo[1,2-d]oxazol-2-ylcarbamate (70 mg, 0.24 mmol) and ammonium chloride (67 mg, 1.2 mmol) in ethanol (2 mL) and water (2 mL) was stirred at 90° C. for 4 h. The reaction mixture was cooled to RT and filtered and rinsed with ethanol. The combined filtrate was concentrated under reduced pressure to afford thiazolo[4′,5′:3,4]benzo[1,2-d]oxazol-2-amine as a yellow solid (30 mg, 65%). LC/MS (ES⁺) calcd for C₈H₅N₃OS: 191.0; found: 192.0 [M+H]. ¹H NMR (400 MHz, DMSO-d6): δ 7.42 (d, J=8.4 Hz, 1H), 7.49 (s, 2H), 7.87 (d, J=8.4 Hz, 1H), 9.41 (s, 1H).

### Preparation of Carboxylic Acid Intermediates

The following amines shown in Table 2 were used in preparing the compounds of the invention. They are either commercially available or can be prepared by known synthetic procedures. CAS registry numbers are provided for each.

TABLE 2
Commercial carboxylic acids

| Int. | | | |
|---|---|---|---|
| No. | Structure | CAS No. | Acid Name |
| 14 | 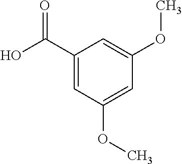 | 1132-21-4 | 3,5-dimethoxy-benzoic acid |
| 15 | 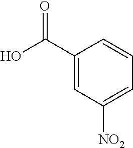 [SMILES: O=C(O)c1cccc([N+](=O)[O-])c1] | 121-92-6 | 3-nitro-benzoic acid |
| 16 | 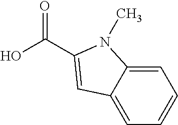 | 16136-58-6 | 1-methyl-1H-Indole-2-carboxylic acid |
| 17 | 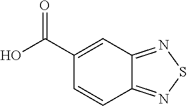 [SMILES: O=C(O)c1ccc2nsnc2c1] | 16405-98-4 | 1,3-benzothiadiazole-5-carboxylic acid |
| 18 | 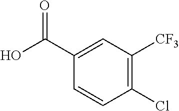 [SMILES: O=C(O)c1ccc(Cl)c(C(F)(F)F)c1] | 1737-36-6 | 4-chloro-3-(trifluoromethyl) benzoic acid |
| 19 | 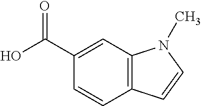 | 202745-73-1 | 1-methyl-1H-Indole-6-carboxylic acid |
| 20 | 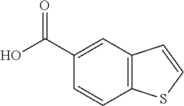 [SMILES: O=C(O)c1ccc2sccc2c1] | 2060-64-2 | benzo[b]thiophene-5-carboxylic acid |
| 21 | 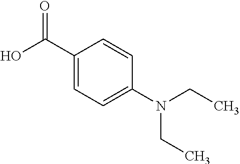 | 5429-28-7 | 4-(diethylamino)-benzoic acid |
| 22 | 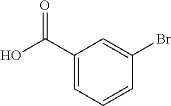 | 585-76-2 | 3-bromo-benzoic acid |
| 23 | 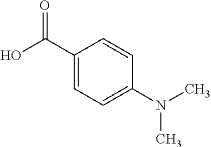 | 619-84-1 | 4-(dimethylamino)-benzoic acid |
| 24 | 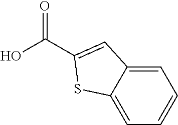 [SMILES: O=C(O)c1cc2ccccc2s1] | 6314-28-9 | benzo[b]thiophene-2-carboxylic acid |
| 25 | 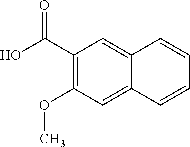 | 883-62-5 | 3-methoxy-2-naphthalenecarboxylic acid |
| 26 | 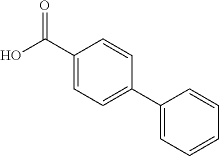 [SMILES: O=C(O)c1ccc(-c2ccccc2)cc1] | 92-92-2 | [1,1′-biphenyl]-4-carboxylic acid |
| 27 | 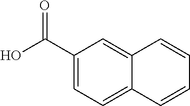 [SMILES: O=C(O)c1ccc2ccccc2c1] | 93-09-4 | 2-naphthalene carboxylic acid |
| 28 | 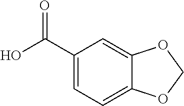 [SMILES: O=C(O)c1ccc2c(c1)OCO2] | 94-53-1 | 1,3-benzodioxole-5-carboxylic acid |
| 29 | 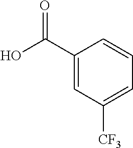 [SMILES: O=C(O)c1cccc(C(F)(F)F)c1] | 454-92-2 | 3-(trifluoromethyl)benzoic acid |
| 30 | 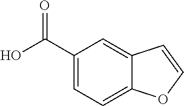 [SMILES: O=C(O)c1ccc2occc2c1] | 90721-27-0 | 1-benzofuran-5-carboxylic acid |
| 31 | 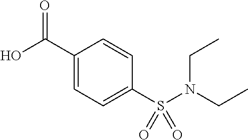 [SMILES: CCN(CC)S(=O)(=O)c1ccc(C(=O)O)cc1] | 1213-06-5 | 4-Diethylsulfamoylbenzoic acid |
| 32 | 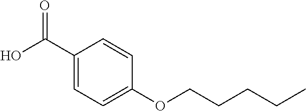 | 15872-41-0 | 4-Pentoxybenzoic acid |
| 33 | 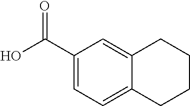 [SMILES: O=C(O)c1ccc2c(c1)CCCC2] | 1131-63-1 | 1,2,3,4-Tetrahydronaphthalene-6- carboxylic acid |

### Intermediate 34: 3-[3-(Morpholin-4-yl)ethoxy]naphthalene-2-carboxylic acid

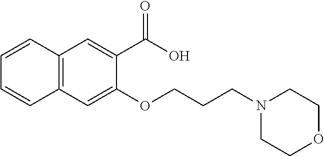 [SMILES: O=C(O)c1cc2ccccc2cc1OCCCN1CCOCC1]

Step 1: To a solution of methyl 3-hydroxy-2-naphthoate (CAS No. 92-70-6, 560 mg, 2.7 mmol), 3-morpholinopropan-1-ol (CAS No. 441-30-9, 800 mg, 5.5 mmol), and PPh₃ (1.44 g, 5.5 mmol) in THF (5.6 mL) at −5° C. was added dropwise DIAD (1.11 g, 5.5 mmol). The resulting mixture was stirred at RT for 12 h. After the solvent was removed, the residue was purified through column chromatography (eluent: DCM/MeOH from 100:1 to 40:1) to afford methyl 3-(3-morpholinopropoxy)-2-naphthoate (732 mg, 80%) as a colorless oil. LC/MS (ES⁺) calcd for C₁₉H₂₃NO₄: 329.5; found: 330.5 [M+H]. ¹H NMR (400 MHz, CDCl₃): δ 8.29 (s, 1H), 7.81 (d, J=8.13 Hz, 1H), 7.71 (d, J=8.22 Hz, 1H), 7.55-7.47 (m, 1H), 7.40-7.33 (m, 1H), 7.19 (s, 1H), 4.19 (t, J=6.13 Hz, 2H), 3.94 (s, 3H), 3.80-3.73 (m, 4H), 2.70 (t, J=7.6 Hz, 2H), 2.64-2.56 (m, 4H), 2.15-2.08 (m, 2H)

Step 2: A solution of methyl 3-(3-morpholinopropoxy)-2-naphthoate (400 mg, 1.2 mmol) and LiOH.H₂O (87 mg, 2.1 mmol) in methanol/water (2 mL/1.6 mL) was stirred at RT for 1 h. The reaction mixture was diluted with water and extracted with ethyl acetate. The aqueous phase was adjusted to pH 6-7 with diluted hydrochloric acid (1.0 N), and extracted with DCM/MeOH (3:1, 4×10 mL). The organic layer was dried over Na₂SO₄ and concentrated under vacuum to afford the 3-[3-(morpholin-4-yl)ethoxy]naphthalene-2-carboxylic acid (240 mg, 63%) as white foam. LC/MS (ES⁺) calcd for C₁₈H₂₁NO₄: 315.3; found: 316.3 [M+H]. ¹H NMR (400 MHz, CDCl₃): δ 8.53 (s, 1H), 7.82 (d, J=8.17 Hz, 1H), 7.71 (d, J=8.24 Hz, 1H), 7.54-7.50 (m, 1H), 7.41-7.37 (m, 1H), 7.23 (s, 1H), 4.35 (t, J=5.94 Hz, 2H), 3.91-3.82 (m, 4H), 2.88 (t, J=6.79 Hz, 2H), 2.81-2.73 (m, 4H), 2.27-2.20 (m, 2H).

### Intermediate 35: 3-[2-(Morpholin-4-yl)ethoxy]naphthalene-2-carboxylic acid

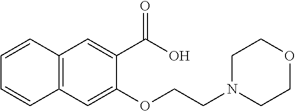 [SMILES: O=C(O)c1cc2ccccc2cc1OCCN1CCOCC1]

This compound can be prepared as described for Intermediate 34 by substituting 3-morpholinopropan-1-ol step 1, with 4-morpholineethanol (CAS No. 622-40-2). LC/MS (ES⁺) calcd for C₁₇H₁₉NO₄: 302.3; found: 303.3 [M+H]. ¹H NMR (400 MHz, CDCl₃): δ 8.43 (d, J=2.2 Hz, 1H), 7.94-7.87 (m, 1H), 7.78-7.72 (m, 1H), 7.56 (ddd, J=8.5, 6.6, 1.1 Hz, 1H), 7.53-7.44 (m, 2H), 4.36 (t, J=6.4 Hz, 2H), 3.69 (t, J=6.0 Hz, 4H), 2.70 (t, J=6.5 Hz, 2H), 2.59-2.44 (m, 4H).

### Intermediate 36: 3-[4-(Morpholin-4-yl)butoxy]naphthalene-2-carboxylic acid

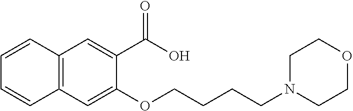 [SMILES: O=C(O)c1cc2ccccc2cc1OCCCCN1CCOCC1]

This compound can be prepared as described for Intermediate 34 by substituting 3-morpholinopropan-1-ol step 1, with 4-morpholinebutanol (CAS No. 5835-79-0). LC/MS (ES⁺) calcd for C₃₉H₂₃NO₄: 329.3; found: 330.4 [M+H]. ¹H NMR (400 MHz, CDCl₃): δ 8.47-8.42 (m, 1H), 7.94-7.87 (m, 1H), 7.75 (dt, J=7.9, 1.9 Hz, 1H), 7.56 (ddd, J=8.5, 6.8, 1.1 Hz, 1H), 7.53-7.42 (m, 2H), 4.05 (t, J=6.1 Hz, 2H), 3.78 (t, J=6.0 Hz, 4H), 2.56-2.43 (m, 6H), 1.77-1.68 (m, 2H), 1.63-1.53 (m, al), 3.78 (t, J=6.0 Hz, 4H), 2.56-2.43 (m, 6H), 1.77-1.68 (m, 2H), 1.63-1.53 (m, 2H).

### Intermediate 37: 3-[2-(Piperidin-1-yl)ethoxy]naphthalene-2-carboxylic acid

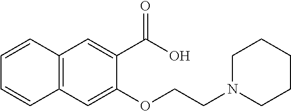 [SMILES: O=C(O)c1cc2ccccc2cc1OCCN1CCCCC1]

This compound be prepared as described above for Intermediate 34 by substituting 3-morpholinopropan-1-ol with 1-piperidineethanol (CAS No. 3040-44-6). LC/MS (ES⁺) calcd for C₁₈H₂₁NO3: 300.3; found: 300.4 [M+H]. ¹H NMR (400 MHz, CDCl₃): δ 8.43 (d, J=2.7 Hz, 1H), 7.94-7.87 (m, 1H), 7.75 (dt, J=8.0, 2.0 Hz, 1H), 7.56 (ddd, J=8.4, 6.7, 1.1 Hz, 1H), 7.53-7.44 (m, 2H), 4.40-4.33 (m, 2H), 2.99 (t, J=6.5 Hz, 2H), 2.54-2.48 (m, 4H), 1.54-1.38 (m, 6H).

### Intermediate 38: 3-[2-(oxan-4-yl)ethoxy]naphthalene-2-carboxylic acid

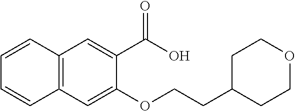 [SMILES: O=C(O)c1cc2ccccc2cc1OCCC1CCOCC1]

This compound can be prepared as described for Intermediate 34 above by substituting 3-morpholinopropan-1-ol with tetrahydro-211-pyran-4-ethanol (CAS No. 4677-18-3). LC/MS (ES⁺) calcd for C₁₈H₂₀O4: 300.3; found: 300.4 [M+H]. ¹H NMR (400 MHz, CDCl₃): δ 8.09-8.04 (m, 1H), 7.91 (dt, J=8.1, 1.7 Hz, 1H), 7.78-7.72 (m, 1H), 7.64 (dd, J=1.8, 0.6 Hz, 1H), 7.56 (ddd, J=8.0, 6.9, 1.3 Hz, 1H), 7.53-7.46 (m, 1H), 4.12 (t, J=6.6 Hz, 2H), 3.76 (ddd, J=12.0, 7.2, 5.0 Hz, 2H), 3.46 (ddd, J=11.9, 7.3, 5.0 Hz, 2H), 1.74 (q, J=6.5 Hz, 2H), 1.73-1.64 (m, 2H), 1.60 (dddd, J=13.4, 7.1, 6.3, 5.0 Hz, 2H), 1.53 (dt, J=12.3, 6.1 Hz, 1H).

### Intermediate 39: 3-(2-(4-(tert-butoxycarbonyl)piperazin-1-yl)ethoxy)-2-naphthoic acid

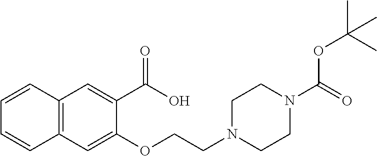 [SMILES: CC(C)(C)OC(=O)N1CCN(CCOc2cc3ccccc3cc2C(=O)O)CC1]

Step 1: To a solution of 2-(piperazin-1-yl)ethanol (1.0 g, 7.7 mmol) in DCM (10 mL) was added (Boc)₂O at RT. After stirring for 1 h, the reaction mixture was diluted with DCM (10 mL) and washed with water (10 mL). The organic phase was dried over Na₂SO₄ and concentrated in vacuo to give a crude residue which was purified by silica gel column chromatography (eluent: DCM/MeOH from 100:1 to 10:1) to afford 1-tert-butyl 4-(2-hydroxyethyl) piperazine-1-carboxylate (1.2 g, 70%) as a colorless oil. LC/MS (ES⁺) calcd for C₁₁H₂₂N₂O₃: 230.3; found: 231.2 [M+H]. ¹H NMR (400 MHz, CDCl₃): δ 3.63 (t, J=5.25 Hz, 2H), 3.53-3.36 (m, 4H), 2.57-2.54 (m, 3H), 2.47-2.44 (m, 4H), 1.46 (s, 9H)

Step 2: DIAD (1.1 g, 5.3 mmol) was added dropwise to a solution of methyl 3-hydroxy-2-naphthoate (530 mg, 2.6 mmol), tert-butyl-4-(2-hydroxyethyl) piperazine-1-carboxylate (1.2 g, 5.3 mmol), and PPh₃ (1.3 g, 5.3 mmol) in THF (5.5 mL) at −5° C. under N₂. The resulting mixture was stirred at RT for 3 h. After the solvent was removed, the residue was purified by silica gel column chromatography (eluent: DCM/MeOH from 100:1 to 40:1) to afford tert-butyl 4-(2-43-(methoxycarbonyl) naphthalen-2-yl)oxy)ethyl) piperazine-1-carboxylate (1.77 g) as a colorless oil. LC/MS (ES⁺) calcd for C₂₃H₃₀N₂O₅: 414.6; found: 415.6 [M+H].

Step 3: A solution of methyl tert-butyl 4-(2-((3-(methoxycarbonyl) naphthalen-2-yl) oxy)ethyl) piperazine-1-carboxylate (1.77 g, 2.6 mmol) and LiOH.H₂O (300 mg, 7.2 mmol) in methanol/water (10 mL/8 mL) was stirred at RT for 1 h. The reaction mixture was diluted with water and extracted with ethyl acetate. The aqueous phase was acidified with hydrochloric acid (1N) to pH 6-7 and extracted with DCM/MeOH (3:1, 4×15 mL). The organic layer was dried over Na₂SO₄ and concentrated in vacuo to afford 3-(2-(4-(tert-butoxycarbonyl)piperazin-1-yl) ethoxy)-2-naphthoic acid (558 mg, 53% yield over two steps) as white foam. LC/MS (ES⁺) calcd for C₂₂H₂₈N₂O₅: 400.6; found: 401.6. ¹H NMR (400 MHz, CDCl₃): δ 8.61 (s, 1H), 7.87 (d, J=8.05 Hz, 1H), 7.73 (d, J=8.23 Hz, 1H), 7.55 (t, J=7.55 Hz, 1H), 7.43 (t, J=7.65 Hz, 1H), 7.30 (s, 1H), 4.42 (t, J=4.82 Hz, 2H), 3.55-3.42 (m, 4H), 2.84 (t, J=5.10 Hz, 2H), 2.54-2.45 (m, 4H), 1.44 (s, 9H)

### Intermediate 40: 3-(2-{2-Oxa-5-azabicyclo[2.2.1]heptan-5-yl}ethoxy)-naphthalene-2-carboxylic acid

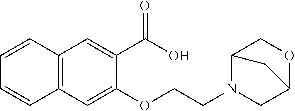 [SMILES: O=C(O)c1cc2ccccc2cc1OCCN1CC2CC1CO2]

This compound can be prepared as described above for Intermediate 34 by substituting 3-morpholinopropan-1-ol with 2-oxa-5-azabicyclo [2.2.1]heptane-5-ethanol (CAS No. 99969-71-8) in step 1. LC/MS (ES⁺) calcd for C₁₈H₁₉N₂O₄: 313.4; found: 314.4 [M+H]. ¹H NMR (400 MHz, CDCl₃): δ 8.43 (dd, J=1.46, 0.69 Hz, 1H), 7.92 (dt, J=7.29, 1.60 Hz, 1H), 7.75 (dt, J=7.57, 1.46 Hz, 1H), 7.56-7.49 (m, 2H), 7.41 (td, J=7.54, 1.62 Hz, 1H), 4.20-4.07 (m, 2H), 3.91 (d, J=6.96 Hz, 2H), 3.77 (p, J=7.04 Hz, 1H), 3.59 (p, J=6.96 Hz, 1H), 3.20 (dd, J=12.45, 6.95 Hz, 1H), 3.11 (dt, J=12.64, 7.14 Hz, 1H), 3.07-2.98 (m, 2H), 2.19-2.05 (m, 2H).

### Intermediate 41: 3-(2-{8-Oxa-3-azabicyclo[3.2.1]octan-3-yl}ethoxy)naphthalene-2-carboxylic acid

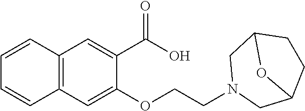 [SMILES: O=C(O)c1cc2ccccc2cc1OCCN1CC2CCC(C1)O2]

This compound can be prepared as described above for Intermediate 34 by substituting 3-morpholinopropan-1-ol with 8-oxa-3-azabicyclo[3.2.1] octane-3-ethanol (CAS No. 99969-71-8). LC/MS (ES⁺) calcd for C₁₉H₁₂₁N₂O₄: 314.4; found: 328.4 [M+H]. ¹H NMR (400 MHz, CDCl₃): δ 8.43 (dd, J=1.47, 0.68 Hz, 1H), 7.92 (dt, J=6.81, 1.19 Hz, 1H), 7.75 (dt, J=7.64, 1.47 Hz, 1H), 7.56-7.49 (m, 2H), 7.43 (td, J=7.48, 1.47 Hz, 1H), 4.17 4.09 (m, 2H), 3.78-3.67 (m, 2H), 3.07 (dd, J=12.45, 6.96 Hz, 2H), 3.01 (td, J=7.04, 1.19 Hz, 2H), 2.92 (dd, J=12.45, 6.96 Hz, 2H), 1.91-1.78 (m, 4H).

### Intermediate 42: 3-(2-{2-Oxa-6-azaspiro[3.3]heptan-6-yl}ethoxy)naphthalene-2-carboxylic acid

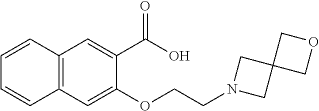 [SMILES: O=C(O)c1cc2ccccc2cc1OCCN1CC2(COC2)C1]

This compound can be prepared as described above for Intermediate 34 by substituting 3-morpholinopropan-1-ol with 2-oxa-6-azaspiro[3.3]heptane-6-ethanol (CAS No. 26096-35-5). LC/MS (ES⁺) calcd for C₁₈H₁₉N₂O₄: 313.4; found: 314.3 [M+H]. ¹H NMR (400 MHz, CDCl₃): δ 8.43 (dd, J=1.48, 0.64 Hz, 1H), 7.92 (dt, J=7.24, 1.54 Hz, 1H), 7.75 (dt, J=7.53, 1.46 Hz, 1H), 7.56-7.49 (m, 2H), 7.41 (td, J=7.52, 1.60 Hz, 1H), 4.08 (t, J=7.09 Hz, 2H), 3.70 (s, 3H), 3.08 (s, 3H), 2.90 (t, J=7.11 Hz, 2H).

### Intermediate 43: 6-[2-(Morpholin-4-yl)ethoxy]-2,1,3-benzothiadiazole-5-carboxylic acid

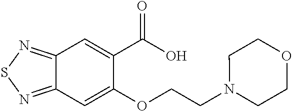 [SMILES: O=C(O)c1cc2nsnc2cc1OCCN1CCOCC1]

Step 1: To a suspension of methyl 4-amino-2-methoxybenzoate (5.0 g, 27.6 mmol) and Et₃N (3.35 g, 33.1 mmol) in DCM (30 mL) was added dropwise acetyl chloride (2.6 g, 33.1 mmol) at 0-5° C. The resulting mixture was stirred at RT for 2 h. The mixture was washed with saturated aq. NaHCO₃, dried over Na₂SO₄ and concentrated. The crude product was purified by column (hexane/ethyl acetate=1/1) to afford methyl 4-acetamido-2-methoxybenzoate as a white solid (5.4 g, 87%). LC/MS (ES⁺): m/z calculated for C₁₁H₁₃NO₄: 223.1; found: 224.1 [M+H]. ¹H NMR (400 MHz, CDCl₃): δ 7.81 (d, J=8.4 Hz, 1H), 7.62 (s, 1H), 7.50 (br, 1H), 6.85-6.82 (dd, J=8.6 Hz, 1.8 Hz, 1H), 3.89 (s, 3H), 3.86 (s, 3H), 2.20 (s, 3H).

Step 2: To a solution of methyl 4-acetamido-2-methoxybenzoate (5.4 g, 24.2 mmol) in acetic acid (50 mL) and Ac₂O (50 mL) was added dropwise HNO₃ (10 mL) at 0-5° C. The resulting mixture was stirred at RT for 12 h. The reaction mixture was poured into ice-water and stirred for 1 h. The precipitate was collected by filtration, washed with water and dried to afford methyl 4-acetamido-2-methoxy-5-nitrobenzoate as a yellow solid (5.8 g, 90%). LC/MS (ES⁺): m/z calculated for C₁₁H₁₂N₂O₆: 268.1; found: 269.1 [M+H]. ¹H NMR (400 MHz, CDCl₃): δ 10.88 (br, 1H), 8.84 (s, 1H), 8.63 (s, 1H), 4.03 (s, 3H), 3.91 (s, 3H), 2.33 (s, 3H).

Step 3: A mixture of methyl 4-acetamido-2-methoxy-5-nitrobenzoate (5.8 g, 21.6 mmol) and K₂CO₃ (6.0 g, 43.2 mmol) in methanol (150 mL) was stirred at RT for 3 h. After methanol was removed, the residue was diluted with water and stirred for 1 h. The precipitates were filtered, washed with water and dried to afford methyl 4-amino-2-methoxy-5-nitrobenzoate as a yellow solid (3.0 g, 60%). LC/MS (ES⁺): m/z calculated for C₉H₁₀N₂O₅: 226.1; found: 227.1 [M+H]. ¹H NMR (400 MHz, CDCl₃): δ 8.50 (s, 1H), 7.85 (br, 2H), 6.54 (s, 1H), 3.82 (s, 3H), 3.74 (s, 3H).

Step 4: A mixture of methyl 4-amino-2-methoxy-5-nitrobenzoate (3.0 g, 13.3 mmol) and Pd/C (0.3 g) in methanol (50 mL) was stirred under hydrogen at 50° C. for 12 h. After Pd/C was filtered, the filtrate was concentrated to afford methyl 4,5-diamino-2-methoxybenzoate as a brown solid (2.6 g, 99%). LC/MS (ES⁺): mlz calculated for C₉H₁₂N₂O₃: 196.1; found: 197.1 [M+H]. ¹H NMR (400 MHz, CDCl₃): δ 7.33 (s, 1H), 6.29 (s, 1H), 3.98 (br, 2H), 3.83 (s, 6H), 3.02 (br, 2H).

Step 5: To a solution of methyl 4,5-diamino-2-methoxybenzoate (2.5 g, 12.7 mmol) and Et₃N (5.16 g, 54 mmol) in DCM (50 mL) was added dropwise SOCl₂ (3.0 g, 25.5 mmol) at 0-5° C. The resulting solution was heated to reflux for 4 h. It was quenched with water and then extracted with DCM. The combined organic layers were washed with 1 M aq. HCl and brine, dried over Na₂SO₄ and concentrated. The crude product was purified by column (hexane/ethyl acetate=10/1) to afford methyl 6-methoxybenzo[c][1,2,5]thiadiazole-5-carboxylate as a white solid (2.5 g, 87%). LC/MS (ES⁺): m/z calculated for C₉H₈N₂O₃S: 224.0; found: 225.0 [M+H]. ¹H NMR (400 MHz, CDCl₃): δ 8.28 (s, 1H), 7.31 (s, 1H), 3.99 (s, 3H), 3.97 (s, 3H).

Step 6: To a solution of methyl 6-methoxybenzo[c][1,2,5]thiadiazole-5-carboxylate (1.0 g, 4.46 mmol) in toluene (20 mL) was added AlCl₃ (1.78 g, 13.4 mmol) slowly. The resulting mixture was heated to reflux for 4 h. The reaction mixture was quenched with ice water and extracted with ethyl acetate. The combined organic layers were washed with water, brine, dried over Na₂SO₄ and concentrated. The crude product was purified by column (hexane/ethyl acetate=20/1) to afford methyl 6-hydroxybenzo[c][1,2,5]thiadiazole-5-carboxylate as a yellow solid (750 mg, 84%). LC/MS (ES⁺): m/z calculated for C₈H₆N₂O₃S: 210.0; found: 211.0 [M+H]. ¹H NMR (400 MHz, CDCl₃): δ 10.65 (s, 1H), 8.72 (s, 1H), 7.45 (s, 1H), 4.08 (s, 3H).

Step 7: A mixture of methyl 6-hydroxybenzo[c][1,2,5]thiadiazole-5-carboxylate (790 mg, 3.76 mmol), 1,2-dibromoethane (7.0 g, 37.6 mmol), and Cs₂CO₃ (2.5 g, 7.52 mmol) in DMF (16 ml) was stirred at RT for 1.5 h. The reaction mixture was quenched with water and extracted with ethyl acetate. The organic layer was washed with brine and concentrated. The crude product was purified through column chromatography (hexane/DCM=1/1) to afford methyl 6-(2-bromoethoxy) benzo [c][1,2,5]thiadiazole-5-carboxylate as white solid (740 mg, 67%). LC/MS (ES⁺): m/z calculated for C₁₀H₁₀N₂O₃SBr: 316.0; found: 317.0 [M+H]. ¹H NMR (400 MHz, CDCl₃): δ 8.30 (s, 1H), 7.30 (s, 1H), 4.45 (t, J=6.4 Hz, 2H), 3.98 (s, 3H), 3.74 (t, J=6.4 Hz, 2H).

Step 8: A solution of methyl 6-(2-bromoethoxy) benzo [c][1,2,5]thiadiazole-5-carboxylate (740 mg, 2.33 mmol) and morpholine (1.5 mL) in toluene (10 ml) was heated at 90° C. for 2 h. The reaction mixture was quenched with water, and extracted with ethyl acetate. The organic layer was washed with brine, dried over Na₂SO₄, and concentrated. The crude product was purified through column chromatography (DCM/MeOH=40/1) to afford methyl 6-(2-morpholinoethoxy)benzo[c][1,2,5]thiadiazole-5-carboxylate as light yellow solid (500 mg, 66%). LC/MS (ES⁺): m/z calculated for C₁₄H₁₇N₃O₄S: 323.4; found: 324.3 [M+H]. ¹H NMR (400 MHz, CDCl₃): δ 8.27 (s, 1H), 7.30 (s, 1H), 4.27 (t, J=5.6 Hz, 2H), 3. 95 (s, 3H), 3.74 (t, J=4.4 Hz, 4H), 2.91 (t, J=5.6 Hz, 2H), 2.63 (t, J=4.4 Hz, 4H).

Step 9: To a solution of 6-(2-morpholinoethoxy)benzo[c][1,2,5]thiadiazole-5-carboxylate (500 mg, 1.55 mmol) in THF/MeOH/H₂O (6 ml/2 mL/2 mL) was added LiOH—H₂O (97 mg, 2.32 mmol). The mixture was stirred at RT for 3 h. HCl (2.3 mL, 1 N) was added and the mixture was concentrated. The crude product was purified by column chromatography (DCM/MeOH=15/1) to afford title product as white solid (610 mg, 128%). LC/MS (ES⁺): m/z calculated for C₁₃H₁₄N₃O₄S: 310.1; found: 310.1 [M+H]. ¹H NMR (400 MHz, DMSO-d6): δ 8.14 (s, 1H), 7.61 (s, 1H), 4.41 (t, J=4.8 Hz, 2H), 3.66 (br, 4H), 2.96 (t, J=4.8 Hz, 2H), 2.73 (br, 4H).

### Intermediate 44: 6-[4-(morpholin-4-yl)butoxy]naphthalene-2-carboxylic acid

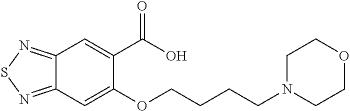 [SMILES: O=C(O)c1cc2nsnc2cc1OCCCCN1CCOCC1]

This compound can be prepared as described above for Intermediate 43 by substituting 1,2-dibromoethane with 1,3-dibromoethane (CAS No. 109-64-8) in step 7. LC/MS (ES⁺): m/z calculated for C₁₅H₁₈N₃O₄S: 336.4; found 337.4 [M+H]. ¹H NMR (400 MHz, CDCl₃): δ 9.07 (s, 1H), 7.95 (s, 1H), 4.00 (t, J=7.05 Hz, 2H), 3.78 (t, J=7.11 Hz, 4H), 2.51 (q, J=6.96 Hz, 6H), 1.83 (p, J=7.10 Hz, 2H), 1.59 (p, J=7.01 Hz, 2H).

### Intermediate 45: 6-[4-(morpholin-4-yl)butoxy]-2,1,3-benzothiadiazole-5-carboxylic acid

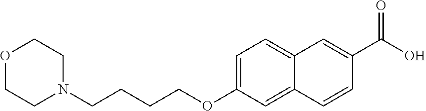 [SMILES: O=C(O)c1ccc2cc(OCCCCN3CCOCC3)ccc2c1]

This compound can be prepared as described above for Intermediate 34 by substituting methyl 3-hydroxy-2-naphthoate with methyl 6-hydroxynaphthalene-2-carboxylate (CAS No. 17295-11-3). LC/MS (ES⁺): m/z calculated for C₁₉H₂₃NO₄: 329.4; found: 330.4 [M+H]. ¹H NMR (400 MHz, CDCl₃): δ 8.36 (t, J=1.58 Hz, 1H), 8.06 (dd, J=7.50, 1.42 Hz, 1H), 7.98 (dd, J=7.58, 1.57 Hz, 1H), 7.82 (dd, J=7.34, 1.60 Hz, 1H), 7.13 (p, J=0.85 Hz, 1H), 7.01 (dd, J=7.58, 1.58 Hz, 1H), 4.04 (t, J=7.05 Hz, 2H), 3.76 (t, J=7.09 Hz, 4H), 2.56-2.48 (m, 6H), 1.77 (p, J=7.07 Hz, 2H), 1.64-1.55 (m, 2H).

### Intermediate 46: 2-[2-(Morpholin-4-yl)ethoxy]-4-phenylbenzoic acid

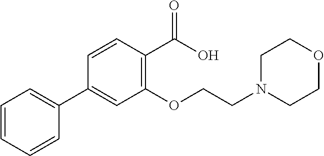 [SMILES: O=C(O)c1ccc(-c2ccccc2)cc1OCCN1CCOCC1]

Step 1: To a solution of methyl 4-bromo-2-methoxybenzoate (CAS No. 139102-34-4, 50 g, 204.02 mmol) and phenylboronic acid (29.85 g, 244.83 mmol) in toluene/EtOH/H₂O (195 ml/50 ml/25 ml) was added Na₂CO₃ (86.5 g, 810.1 mmol) and Pd(PPh₃)₄ (4.7 g, 4.1 mmol) under nitrogen atmosphere. The resulting mixture was heated to 100° C. under nitrogen atmosphere and stirred for 4 h. After the completion of the reaction, the reaction mixture was filtered through celite, and the filter cake was rinsed with ethyl acetate. The organic phase was collected, and the aqueous phase was extracted with ethyl acetate. The combined organic phases were dried over Na₂SO₄ and concentrated under reduced pressure to give a residue which was purified through silica gel flash column chromatography (eluent: hexane/DCM=2/1˜1/1) to afford methyl 3-methoxy-[1,1′-biphenyl]-4-carboxylate as a yellow solid (49.22 g, 91%). LC/MS (ES⁺) calcd for C₁₅H₁₄O₃: 242.1; found: 243.0 [M+H]. ¹H NMR (400 MHz, DMSO-d6): δ 7.78-7.72 (m, 1H), 7.53-7.46 (m, 2H), 7.46-7.40 (m, 1H), 7.36 (d, J=1.2 Hz, 1H), 7.30 (dd, J=1.2 Hz, 12.0 Hz, 1H), 3.93 (s, 3H), 3.80 (s, 3H).

Step 2: To a solution of methyl 3-methoxy-[1,1′-biphenyl]-4-carboxylate (49.2 g, 203.1 mmol) in DCM (200 ml) was added dropwise a solution of BBr₃ (137.8 g, 550 mmol) in DCM (250 ml) with dry ice-acetone bath. The resulting mixture was stirred at −70° C. for 10 min, and then quenched with methanol (100 ml) slowly. The reaction mixture was washed with water (300 ml), and the aqueous phase was extracted with DCM. The combined organic phases were washed with brine, dried over Na₂SO₄, and concentrated under reduced pressure to give a residue which was purified through silica gel flash column chromatography (eluent: hexane/DCM=2/1) to afford methyl 3-hydroxy-[1,1′-biphenyl]-4-carboxylate as a white solid (44.62 g, 96%). LC/MS (ES⁺) calcd for C₁₄H₁₂O₃: 228.1; found: 229.0 [M+H]. ¹H NMR (400 MHz, DMSO-d6): δ 10.59 (s, 1H), 7.88-7.84 (m, 1H), 7.74-7.69 (m, 2H), 7.52-7.46 (m, 2H), 7.45-7.40 (m, 1H), 7.29-7.25 (m, 2H), 3.91 (s, 3H).

Step 3: To a stirred solution of methyl 3-hydroxy-[1,1′-biphenyl]-4-carboxylate (14.46 g, 63.35 mmol) and 4-(2-chloroethyl)morpholine HCl salt (14.06 g, 76.0 mmol) in DMF (240 mL) was added Cs₂CO₃ (61.9 g, 190.1 mmol). The resulting mixture was stirred at 85° C. under nitrogen atmosphere for 3 h. The reaction mixture was cooled down to RT and filtered. The filter cake was rinsed with ethyl acetate. The combine organic phase was washed with water and then brine, dried over Na₂SO₄, and concentrated under reduced pressure to give a residue which was purified through silica gel flash column chromatography (eluent: DCM/ethyl acetate=5/1) to afford methyl 3-(2-morpholinoethoxy)-[1,1′-biphenyl]-4-carboxylate as a yellow oil (21.69 g, 100%). LC/MS (ES⁺) calcd for C₂₀H₂₃NO₄: 341.2; found: 342.4 [M+H]. ¹H NMR (400 MHz, CDCl₃): δ 7.87 (d, J=8.0 Hz, 1H), 7.61-7.56 (m, 2H), 7.48-7.42 (m, 2H), 7.42-7.36 (m, 1H), 7.21 (dd, J=1.6 Hz, 8.0 Hz, 1H), 7.17 (d, J=1.6 Hz, 1H), 4.26 (t, J=5.8 Hz, 2H), 3.89 (s, 3H), 3.76-3.71 (m, 4H), 2.89 (t, J=5.6 Hz, 1H), 2.67-2.60 (m, 4H).

Step 4: To a solution of methyl 3-(2-morpholinoethoxy)-[1,1′-biphenyl]-4-carboxylate (24.46 g, 71.6 mmol) in THF/MeOH/H₂O (140 ml/40 ml/40 ml) was added NaOH (7.1 g, 179 mmol). After stirring at RT for 2 h, THF and methanol were removed under reduced pressure, and the aqueous phase was acidified with hydrochloric acid (1 N. The precipitate formed was collected through filtration, washed with watered, ried to give 3-(2-morpholinoethoxy)-[1,1′-biphenyl]-4-carboxylic acid as a white solid (22.8 g, 88%). LC/MS (ES⁺) calcd for C₁₉H₂₁NO₄: 327.2; found: 328.3 [M+H]. ¹H NMR (400 MHz, DMSO-d6): δ 12.09 (br, 2H), 7.81 (d, J=8.4 Hz, 1H), 7.79-7.74 (m, 2H), 7.54-7.48 (m, 2H), 7.46-7.41 (m, 2H), 7.38 (dd, J=1.6 Hz, 8.0 Hz, 1H), 4.65 (t, J=4.8 Hz, 2H), 3.96-3.84 (m, 4H), 3.61-3.56 (m, 2H), 3.37-3.20 (m, 4H).

### Intermediate 47: 2-[4-(morpholin-4-yl)ethoxy]-4-phenylbenzoic acid

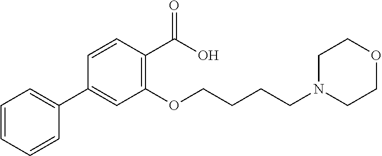 [SMILES: O=C(O)c1ccc(-c2ccccc2)cc1OCCCCN1CCOCC1]

This compound can be prepared as described above for Intermediate 34 by substituting 4-(2-chloroethyl)morpholine with 4-(4-chlorobutyl)-morpholine (CAS No. 734495-59-1) step 3. LC/MS (ES⁺) calcd for C₂₁H₂₅NO₄: 355.4; found: 355.5 [M+H]. ¹H NMR (400 MHz, DMSO-d6): δ 7.82 (d, J=7.49 Hz, 1H), 7.67-7.61 (m, 2H), 7.54 (dd, 7.50, 1.45 Hz, 1H), 7.50-7.43 (m, 2H), 7.43-7.35 (m, 1H), 7.31 (d, J=1.46 Hz, 1H), 4.02 (t, J=7.08 Hz, 2H), 3.59 (t, J=7.08 Hz, 4H), 2.61 (t, J=7.10 Hz, 2H), 2.47 (t, J=7.11 Hz, 4H), 1.80 (p, J=7.12 Hz, 2H), 1.58 (p, J=7.23 Hz, 2H).

### Intermediate 48: 6-[2-(Morpholin-4-yl)ethoxy]-2H-1,3-benzodioxole-5-carboxylic acid

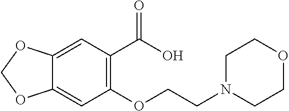 [SMILES: O=C(O)c1cc2c(cc1OCCN1CCOCC1)OCO2]

Step 1: A solution of benzo[d][1,3]dioxole-5-carboxylic acid (CAS No. 326-56-7, 15 g, 90.3 mmol) and concentrated sulfuric acid (0.1 mL) in methanol (200 mL) was stirred at 70° C. under nitrogen for 12 h. After completion of the reaction, the reaction mixture was cooled to RT, and concentrated under reduced pressure. The residue was diluted with water, neutralized with saturated aqueous Na₂CO₃ solution, and extracted with ethyl acetate. The combined organic layers were washed with brine, dried over sodium sulfate, and concentrated under reduced pressure to afford methyl benzo[d][1,3]dioxole-5-carboxylate as a white solid (16.0 g, 98%). LC/MS (ES⁺) calcd for C₉H₈O₄: 180.0; found: 181.0 [M+H]. ¹H NMR (400 MHz, DMSO-d6): δ 7.56 (dd, J=1.2, 8.0 Hz, 1H), 7.38 (d, J=0.8 Hz, 1H), 7.03 (d, J=8.4 Hz, 1H), 6.14 (s, 2H), 3.80 (s, 3H).

Step 2: To a stirred solution of methyl benzo[d][1,3]dioxole-5-carboxylate (16 g, 88.8 mmol) in acetic acid (100 mL) was added dropwise fuming nitric acid (111.5 g, 1.7 mol) at 20-25° C. under nitrogen. The resulting mixture was stirred at 20° C. for 30 min. After completion of the reaction, the reaction mixture was poured into ice-water. The precipitate was collected through filtration, washed with water, and dried to afford methyl 6-nitrobenzo[d][1,3]dioxole-5-carboxylate as a yellow solid (19.3 g, 97%). LC/MS (ES⁺) calcd for C₉H₇NO₆: 225.0; found: 226.1 [M+H]. ¹H NMR (400 MHz, DMSO-d6): δ 7.70 (s, 1H), 7.34 (s, 1H), 6.30 (s, 2H), 3.81 (s, 3H).

Step 3: A mixture of methyl 6-nitrobenzo[d][1,3]dioxole-5-carboxylate (19.3 g, 85.7 mmol) and Pd/C (10%, 1.9 g) in ethyl acetate/methanol_(200 mL/100 mL) was stirred at 50° C. under hydrogen atmosphere (hydrogen balloon) for 12 h. After this time, the Pd/C was removed through celite and washed with methanol. The combined filtrate was concentrated under reduced pressure to afford methyl 6-aminobenzo[d][1,3]dioxole-5-carboxylate as an off-white solid (15 g, 90%). LC/MS (ES⁺) calcd for C₉H₉NO₄: 195.1; found: 196.1 [M+H]. ¹H NMR (400 MHz, DMSO-d6): δ 7.07 (s, 1H), 6.66 (s, 2H), 6.35 (s, 1H), 5.93 (s, 2H), 3.72 (s, 3H).

Step 4: A mixture of sodium nitrite (3.9 g, 56.4 mmol) in water (25 mL) was added to a cooled (with an ice bath) mixture of methyl 6-aminobenzo[d][1,3]dioxole-5-carboxylate (11 g, 56.4 mmol) and concentrated sulfuric acid (12 mL) in water (60 mL). The resulting mixture was stirred at 0° C. for 15 minutes. After diluting with water (60 mL), the mixture was added into a boiling solution of cupric sulfate pentahydrate (56.4 g, 225.6 mmol) in water (130 mL). The resulting mixture was refluxed for 10 min, and then cooled down to RT with an ice-bath. The reaction mixture was extracted with ethyl acetate (100 ml×2). The combined organic layer was washed with brine, dried over sodium sulfate, and concentrated under reduced pressure to give a crude product which was purified through silica gel flash column chromatography (hexane/ethyl acetate=50/1) to afford methyl 6-hydroxybenzo[d] [1,3]dioxole-5-carboxylate as a white solid (7.5 g, 68%). LC/MS (ES⁺) calcd for C₉H₈O₅: 196.0; found: 197.0 [M+H]. ¹H NMR (400 MHz, DMSO-d6): δ 10.90 (s, 1H), 7.17 (s, 1H), 6.62 (s, 1H), 6.07 (s, 2H), 3.86 (s, 3H).

Step 5: To a mixture of methyl 6-hydroxybenzo[d][1,3]dioxole-5-carboxylate (3.0 g, 15.3 mmol) and cesium carbonate (10.0 g, 30.6 mmol) in DMF (50 mL) was added 1,2-dibromoethane (14.3 g, 76.5 mmol). The resulting mixture was stirred at 85° C. under nitrogen for 12 h. After completion of the reaction, the reaction mixture was cooled to RT and filtered. The filtrate was diluted with ethyl acetate (200 ml), washed with water (300 ml×2) and then brine (100 ml), dried over sodium sulfate, and concentrated under reduced pressure to give a crude product which was purified through silica gel flash column chromatography (hexane/ethyl acetate=20/1) to afford methyl 6-(2-bromoethoxy) benzo[d][1,3]dioxole-5-carboxylate as a white solid (1.5 g, 32%). LC/MS (ES⁺) calcd for C₁₁H₁₁BrO₅: 302.0; found: 305.1 [M+3]. ¹H NMR (400 MHz, DMSO-d6): δ 7.18 (s, 1H), 6.89 (s, 1H), 6.06 (s, 2H), 4.01 (t, J=6.0 Hz, 2H), 3.73 (s, 3H), 3.62 (t, J=6.8 Hz, 2H), 2.04-1.96 (m, 2H), 1.84-1.76 (m, 2H).

Step 6: A solution of methyl 6-(2-bromoethoxy)benzo[d][1,3]dioxole-5-carboxylate (1.5 g, 4.9 mmol) and morpholine (8.5 g, 98.0 mmol) in toluene (20 mL) was stirred at 100° C. 12 h. After completion of the reaction, the reaction mixture was cooled to RT, and concentrated under reduced pressure to give a residue which was purified through silica gel flash column chromatography (hexane/ethyl acetate=1/1) to afford methyl 6-(2-morpholino ethoxy)benzo[d][1,3]dioxole-5-carboxylate as a yellow oil (1.5 g, 98%). LC/MS (ES⁺) calcd for C₁₅H₁₉NO₆: 309.1; found: 310.3 [M+H]. ¹H NMR (400 MHz, DMSO-d6): δ 7.16 (s, 1H), 6.91 (s, 1H), 6.06 (s, 2H), 4.08 (t, J=5.6 Hz, 2H), 3.72 (s, 3H), 3.56 (t, J=4.4 Hz, 4H), 2.66 (t, J=5.6 Hz, 2H), 2.49-2.46 (m, 4H).

Step 7: To a stirred solution of methyl 6-(2-morpholinoethoxy)benzo[d][1,3]dioxole-5-carboxylate (1.5 g, 4.8 mmol) in methanol/water (1/1, 20 mL) was added LiOH—H₂O (1 g, 24.2 mmol). The resulting mixture was stirred at RT for 12 h. After completion of the reaction, the methanol was removed under reduced pressure, and the residue was acidified with diluted hydrochloric acid (IN) to pH 5-6. After concentration under reduced pressure, the crude product was purified through silica gel flash column chromatography (DCM/MeOH=10/1) to afford 6-(2-morpholinoethoxy)benzo[d][1,3]dioxole-5-carboxylic acid as an off-white solid (1.4 g, 98%). LC/MS (ES⁺) calcd for C₁₄H₁₇NO₆: 295.1; found: 296.3 [M+H]. ¹H NMR (400 MHz, DMSO-d6): δ 12.40 (br, 1H), 7.20 (s, 1H), 6.98 (s, 1H), 6.07 (s, 2H), 4.48 (t, J=4.8 Hz, 2H), 3.89 (t, J=4.8 Hz, 4H), 3.55-3.47 (m, 6H).

### Intermediate 49: 6-[4-(morpholin-4-yl)butoxy]-2H-1,3-benzodioxole-5-carboxylic acid

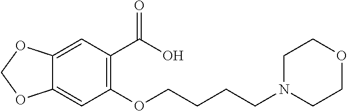 [SMILES: O=C(O)c1cc2c(cc1OCCCCN1CCOCC1)OCO2]

This compound can be prepared as described above for Intermediate 48, 6-[2-(morpholin-4-yl)ethoxy]-2H-1,3-benzodioxole-5-carboxylic acid by substituting 1,2-dibromoethane with 1,2-dibromobutane in step 5. LC/MS (ES⁺) calcd for C₁₆H₂₁NO₆: 323.3; found: 324.4 [M+H]. 1H NMR (400 MHz, DMSO-d6): δ 7.56 (s, 1H), 6.71 (s, 1H), 6.06 (s, 1H), 4.03 (t, J=7.11 Hz, 1H), 3.59 (t, J=7.09 Hz, 2H), 2.60 (t, J=7.07 Hz, 1H), 2.46 (t, J=7.11 Hz, 2H), 1.82 (p, J=6.99 Hz, 1H), 1.58 (p, J=7.10 Hz, 1H).

### Intermediate 50: 6-[2-(morpholin-4-yl)ethoxy]-1-benzothiophene-5-carboxylic acid

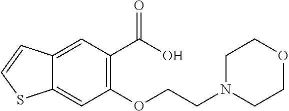 [SMILES: O=C(O)c1cc2ccsc2cc1OCCN1CCOCC1]

Step 1: To a solution of Br₂ (50 g, 0.311 mol) and KBr (92.6 g, 0.779 mol) in water (480 mL) was added 2-fluoro-4-methoxybenzaldehyde (CAS No. 331-64-6, 24 g, 0.16 mol) in portions at 0° C. The resulting mixture was stirred at RT for 4 h. The reaction mixture was filtered, and the filter cake was washed with water and dried to afford 5-bromo-2-fluoro-4-methoxybenzaldehyde as a white solid (28.9 g, 80%). LC/MS (ES⁺) calcd for C₈H₆BrFO₂: 232.0; found: 233.0 [M+H].

Step 2: To a mixture of 5-bromo-2-fluoro-4-methoxybenzaldehyde (20 g, 86 mmol) and K₂CO₃ (17.8, 129 mmol) in DMF (200 mL) was added methyl 2-mercaptoacetate (9.6 g, 90 mmol). The resulting mixture was stirred at 60° C. under N₂ for 30 min. The reaction mixture was quenched with water, and the precipitate formed was filtered. The filter cake was washed with water and dried to afford methyl 5-bromo-6-methoxybenzo[b]thiophene-2-carboxylate as a white solid (16.2 g, 63%). LC/MS (ES⁺) calcd for C₁₁H₉BrO₃S: 300.0; found: 300.9 [M+H]. ¹H NMR (400 MHz, DMSO-d6): δ 8.29 (s, 1H), 8.08 (s, 1H), 7.81 (s, 1H), 3.94 (s, 3H), 3.87 (s, 3H).

Step 3: To a solution of methyl 5-bromo-6-methoxybenzo[b]thiophene-2-carboxylate (15 g, 49.8 mmol) in THE (200 mL) and water (80 mL) was added LiOH—H₂O (20.9 g, 498 mmol). The resulting mixture was stirred at 50° C. under N₂ for 3 h. The reaction mixture was cooled to RT, and acidified with hydrochloric acid (2 N) under ice-water bath. The precipitate formed was filtered and dried to afford 1-(2-aminobenzo[d]thiazol-7-yl)-3-phenylthiourea as a white solid (13.6 g, 95%). LC/MS (ES⁺) calcd for C₁₀H₇BrO₃S: 286.0; found: 286.9 [M+H]. ¹H NMR (400 MHz, DMSO-d6): δ 8.26 (s, 1H), 7.98 (s, 1H), 7.80 (s, 1H), 3.93 (s, 3H).

Step 4: To a suspension of 5-bromo-6-methoxybenzo[b]thiophene-2-carboxylic acid (20.7 g, 72 mmol) in quinoline (200 mL) was added copper powder (8.0 g, 126 mmol). The resulting mixture was stirred at 190° C. under N₂ for 3 h. After cooling to RT, the mixture was diluted with water, and acidified with hydrochloric acid (4 N) to adjust the pH to 3-4. The aqueous phase was extracted with ethyl acetate (80 mL×3); the combined organic phase was washed with brine, dried over Na₂SO₄, and concentrated under reduced pressure to give a residue which was purified through silica gel flash column chromatography (hexane/ethyl acetate=20/1) to afford 5-bromo-6-methoxybenzo [b]thiophene as a brown solid (11.3 g, 64%). LC/MS (ES⁺) calcd for C₉H₇BrOS: 241.9; found: 244.9 [M+H]. ¹H NMR (400 MHz, CDCl₃): δ 7.96 (s, 1H), 7.33 (s, 1H), 7.28 (d, J=5.6 Hz, 1H), 7.16 (d, J=5.2 Hz, 1H), 3.94 (s, 3H).

Step 5: To a solution of 5-bromo-6-methoxybenzo[b]thiophene (5.0 g, 20.6 mmol), diethyl oxalate (6.0 g, 41.1 mmol), and DMAP (7.5 g, 61.7 mol) in NMP (60 mL) was added Pd(PPh₃)₂Cl₂ (1.5 g, 2.1 mmol). The resulting mixture was stirred at 155° C. under N₂ for 12 h. After cooling to RT, the reaction mixture was diluted with ethyl acetate (200 mL) and filtered through celite. The filtrate was washed with water (300 mL×2) and brine (100 mL), dried over Na₂SO₄, and concentrated under reduced pressure to give a residue which was purified through silica gel flash column chromatography (hexane/ethyl acetate=20/1) to afford ethyl 6-methoxybenzo[b]thiophene-5-carboxylate as a yellow solid (2.4 g, 49%). LC/MS (ES⁺) calcd for C₁₂H₁₂O₃S: 236.1; found: 237.1 [M+H]. ¹H NMR (400 MHz, CDCl₃): δ 8.24 (s, 1H), 7.41 (s, 1H), 7.30 (d, J=5.6 Hz, 1H), 7.28 (d, J=5.6 Hz, 1H), 4.40 (q, J=7.4 Hz, 2H), 3.96 (s, 3H), 1.41(t, J=7.4 Hz, 3H).

Step 6: To a solution of ethyl 6-methoxybenzo[b]thiophene-5-carboxylate (3.3 g, 14.0 mmol) in dichloromethane (30 mL) was added dropwise a solution of BBr₃ (8.7 g, 34.9 mmol) in dichloromethane (20 mL) with dry ice-acetone bath. The resulting mixture was stirred at −70° C. under N₂ for 1 h. The reaction was quenched with methanol slowly at −10° C., and stirred at the same temperature for 30 min. The reaction mixture was partitioned between DCM and water. The organic phase was collected, and the aqueous phase was extracted with DCM. The combined organic phase was dried over Na₂SO₄ and concentrated under reduced pressure to give a residue which was purified through silica gel flash column chromatography (hexane/ethyl acetate=50/1) to afford ethyl 6-hydroxybenzo[b]thiophene-5-carboxylate as a white solid (2.3 g, 74%). LC/MS (ES⁺) calcd for C₁₁H₁₀O₃S: 222.0; found: 223.0 [M+H]. ¹H NMR (400 MHz, DMSO-d6): δ 10.59 (s, 1H), 8.37 (s, 1H), 7.61-7.58 (m, 2H), 7.46 (d, J=5.2 Hz, 1H), 4.41 (q, J=7.0 Hz, 2H), 1.38 (t, J=7.0 Hz, 3H).

Step 7: To a mixture of ethyl 6-hydroxybenzo[b]thiophene-5-carboxylate (2.0 g, 9 mmol) and 4-(2-chloroethyl)morpholine HCl salt (2.0 g, 10.8 mmol) in DMF (20 mL) was added Cs₂CO₃ (8.8 g, 27 mmol) at RT. The resulting mixture was heated to 85° C., and stirred for 3 h. The reaction mixture was cooled down to RT and filtered; the filtrate was diluted with ethyl acetate (80 mL), washed with water (100 mL×3) and brine (60 mL), dried over Na₂SO₄, and concentrated under reduced pressure to give a residue which was purified through silica gel flash column chromatography (DCM/MeOH=50/1) to afford ethyl 6-(2-morpholinoethoxy) benzo[b]thiophene-5-carboxylate as an off-white solid (2.79 g, 92%). LC/MS (ES⁺) calcd for C₁₇H₂₁NO₄S: 335.1; found: 336.4 [M+H]. ¹H NMR (400 MHz, CDCl₃): δ 8.22 (s, 1H), 7.41 (s, 1H), 7.31 (d, J=5.6 Hz, 1H), 7.28 (d, J=5.6 Hz, 1H), 4.38 (q, J=7.2 Hz, 2H), 4.23 (t, J=5.8 Hz, 2H), 3.76-3.71 (m, 4H), 2.89 (t, J=5.8 Hz, 2H), 2.65-2.60 (m, 4H), 1.40 (t, J=7.2 Hz, 3H).

Step 8: To a solution of ethyl 6-(2-morpholinoethoxy)benzo[b]thiophene-5-carboxylate (2.7 g, 8.3 mmol) in THF/MeOH/H₂O (4:1:1, 30 mL) was added LiOH—H₂O (2.1 g, 50 mmol) at RT. The resulting mixture was stirred at 60° C. for 3 h. THF and MeOH were removed under reduced pressure, and the residue was neutralized with HOAc to adjust the pH to 6. The resulting mixture was extracted with DCM-MeOH mixture (10:1 V/V); the combined organic phase was washed with brine, dried over Na₂SO₄, and concentrated under reduced pressure to give a residue which was triturated with diethyl ether to afford 6-[2-(morpholin-4-yl)ethoxy]-1-benzothiophene-5-carboxylic acid as a white solid (1.92 g, 75%). LC/MS (ES⁺) calcd for C₁₅H₁₇NO₄S: 307.1; found: 308.1 [M+H]. ¹H NMR (400 MHz, DMSO-d6): δ 8.22 (s, 1H), 7.85 (s, 1H), 7.67 (d, J=5.6 Hz, 1E1), 7.45 (d, J=5.6 Hz, 1H), 4.57-4.52 (m, 2H), 3.89-3.84 (m, 4H), 3.62-3.57 (m, 2H), 3.37-3.26 (m, 4H).

### Intermediate 51: 6-[4-(morpholin-4-yl)butoxy]-1-benzothiophene-5-carboxylic acid

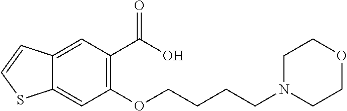 [SMILES: O=C(O)c1cc2ccsc2cc1OCCCCN1CCOCC1]

This compound can be prepared as described above for Intermediate 50, 6-[2-(morpholin-4-yl)ethoxy]-1-benzothiophene-5-carboxylic acid by substituting 4-(2-chloroethyl)morpholine HCl salt with 4-(4-chlorobutyl)-morpholine (CAS No. 734495-59-1) in step 7. LC/MS (ES⁺) calcd for C₁₇H₂₁NO₄S: 335.4; found: 336.4 [M+H]. ¹H NMR (400 MHz, DMSO-d6): δ 8.37 (d, J=1.79 Hz, 1H), 7.57 (dd, J=7.55, 1.44 Hz, 1H), 7.49 (d, J=7.41 Hz, 1H), 7.42 (s, 1H), 4.03 (t, J=7.13 Hz, 2H), 3.59 (t, J=7.09 Hz, 4H), 2.60 (t, J=7.11 Hz, 2H), 2.47 (t, J=7.09 Hz, 4H), 1.84 (p, J=7.04 Hz, 2H), 1.58 (p, J=7.04 Hz, 2H).

### Intermediate 52: 1-methyl-5-[2-(morpholin-4-yl)ethoxy]-1H-indole-6-carboxylic acid

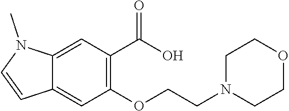 [SMILES: Cn1ccc2cc(OCCN3CCOCC3)c(C(=O)O)cc21]

Step 1: To a mixture of 2-hydroxy-4-methylbenzoic acid (80 g, 0.5 mol) and K₂CO₃ (218 g, 1.58 mol) in DMF (300 mL) was added iodomethane (224 g, 1.5 mol) dropwise at 0° C. The resulting mixture was stirred at 40° C. for 12 h. The reaction mixture was filtered, and the filtrate was partitioned into water (1,500 mL) and ethyl acetate (800 mL). The organic layer was collected, washed with water (300 mL×2) and brine (300 mL), dried over Na₂SO₄, and concentrated under reduce pressure to give a crude product which was purified through silica gel flash column chromatography (cyclohexane/ethyl acetate=10/1) to afford methyl 2-methoxy-4-methylbenzoate as a yellow oil (82 g, 86%). LC/MS (ES⁺) calcd for C₁₀H₁₂O₃: 180.1; found:181.0 [M+H]. ¹H NMR (400 MHz, CDCl₃): δ 7.72 (d, J=8.0 Hz, 1H), 6.78-6.79 (m, 2H), 3.89 (s, 3H), 3.86 (s, 3H), 2.38 (s, 3H).

Step 2: To a mixture of methyl 2-methoxy-4-methylbenzoate (82 g, 0.46 mol) in acetic acid/acetic anhydride (1/1, 400 mL) was added nitric acid (128 mL) dropwise at 0° C. The mixture was then raised to 40° C. slowly and stirred for 12 h. The resulting mixture was poured into ice water and extracted with DCM. The organic phases were washed with brine, dried over Na₂SO₄ and concentrated under reduce pressure. The crude product was purified through silica gel flash column chromatography (cyclohexane/DCM/ethyl acetate=8/2/1) to afford methyl 2-methoxy-4-methyl-5-nitrobenzoate as an off-white solid (65 g, 63%). ¹H NMR (400 MHz, CDCl₃): δ 8.62 (s, 1H), 6.86 (s, 1H), 4.00 (s, 3H), 3.91 (s, 3H), 2.71 (s, 3H).

Step 3: A mixture of methyl 2-methoxy-4-methyl-5-nitrobenzoate (65 g, 0.29 mol) and DMF-DMA (103,7 g, 0.87 mol) in DMF (50 mL) was heated to 115° C., and stirred for 3 h. The reaction mixture was concentrated under reduced pressure to give a crude product which was triturated with diethyl ether to afford methyl 4-(2-(dimethylamino)vinyl)-2-methoxy-5-nitrobenzoate as a red solid (73 g, 90%). ¹H NMR (400 MHz, DMSO-d₆): δ 8.58 (s, 1H), 7.09 (d, J=13.6 Hz, 1H), 6.82 (s, 1H), 6.12 (d, J=13.6 Hz, 1H), 3.98 (s, 3H), 3.87 (s, 3H), 3.00 (s, 6H).

Step 4: A mixture of methyl 4-(2-(dimethylamino)vinyl)-2-methoxy-5-nitrobenzoate (43 g, 0.15 mol) and 10% Pd/C (4.3 g) in THF (80 mL) was stirred at room temperature under hydrogen atmosphere (balloon pressure) for 12 h. After this time, the Pd/C was filtered off, and the filter cake was rinsed with methanol. The combined filtrate was concentrated under reduce pressure to give a crude product that was purified through silica gel flash column chromatography (cyclohexane/DCM/ethyl acetate=8/2/1) to afford methyl 5-methoxy-1H-indole-6-carboxylate as a white solid (21.9 g, 69%). LC/MS (ES⁺) calcd for C₁₁H₁₁NO₃: 205.1; found: 206.0 [M+H]. ¹H NMR (400 MHz, CDCl₃): δ 8.35 (br, 1H), 7.94 (s, 1H), 7.33-7.31 (m, 1H), 7.16 (s, 1H), 6.51-6.48 (m, 1H), 3.93 (s, 3H), 3.91 (s, 3H).

Step 5: A mixture of methyl 5-methoxy-1H-indole-6-carboxylate (21.9 g, 0.1 mol), MeONa (5.9 g, 0.11 mol), and MeI (16.5 g, 0.11 mol) in THF (50 mL) was stirred at 0° C. for 2 h. After completion, the reaction was quenched with water, and extracted with DCM, dried over Na₂SO₄, and concentrated under reduce pressure to give a crude product which was purified through silica gel flash column chromatography (cyclohexane/DCM/ethyl acetate=8/2/1) to afford methyl 5-methoxy-1-methyl-1H-indole-6-carboxylate as a white solid (20.6 g, 88%). LC/MS (ES⁺) calcd for C₁₂H₁₃NO₃: 219.1; found: 220.0 [M+H]. ¹H NMR (400 MHz, CDCl₃): δ 7.87 (s, 1H), 7.15 (d, J=2.8, 1H), 7.14 (s, 1H), 6.40 (dd, J=0.8 Hz, 2.8 Hz, 1H), 3.93 (d, J=1.6 Hz, 6H), 3.80 (s, 3H).

Step 6: To a solution of methyl 5-methoxy-1-methyl-1H-indole-6-carboxylate (7 g, 30 mmol) in DCM (50 mL) was added dropwise BBr₃ in DCM (1.0 N, 150 ml, 150 mmol) at −70° C. under nitrogen atmosphere. After stirring at −70° C. for 30 min, the reaction was quenched slowly with methanol (30 mL) at −70° C., and then warmed to room temperature, and stirred for an additional 30 min. The reaction mixture was partitioned between water and DCM, the organic phase was collected, and the aqueous phase was extracted with DCM (100 mL×2). The combined organic layer was washed with brine, dried over Na₂SO₄, and concentrated under reduce pressure to give a crude product which was purified through silica gel flash column chromatography (cyclohexane/ethyl acetate=10/1) to afford methyl 5-hydroxy-1-methyl-1H-indole-6-carboxylate as a white solid (1.6 g, 22%). LC/MS (ES⁺) calcd for C₁₁H₁₁NO₃: 205.1; found: 206.0 [M+H].

Step 7: A mixture of methyl 5-hydroxy-1-methyl-1H-indole-6-carboxylate (1.6 g, 7.8 mmol), 4-(2-chloroethyl)morpholine hydrochloride (1.7 g, 9.4 mmol), and cesium carbonate (7.6 g, 23.4 mmol) in DMF (20 mL) was stirred at 85° C. under nitrogen atmosphere for 3 h. The reaction mixture was filtered, and the filter cake was rinsed with ethyl acetate. The combined filtrate was washed with water and then brine, dried over Na₂SO₄, and concentrated under reduce pressure to give a crude product that was purified through silica gel flash column chromatography (DCM/MeOH/Et₃N=100/1/5%) to afford methyl 1-methyl-5-(2-morpholinoethoxy)-1H-indole-6-carboxylate as a white solid (2.1 g, 85%). LC/MS (ES⁺) calcd. for C₁₇H₂₂N₂O₄: 318.2; found: 319.3 [M+H]. ¹H NMR (400 MHz, CDCl₃): δ 7.86 (s, 1H), 7.16-7.14 (m, 2H), 6.41-6.38 (m, 1H), 4.21 (t, J=5.6 Hz, 2H), 3.91 (s, 3H), 3.80 (s, 3H), 3.77-3.73 (m, 4H), 2.88 (t, J=5.6 Hz, 2H), 2.66-2.62 (m, 4H).

Step 8: To a solution of methyl 1-methyl-5-(2-morpholinoethoxy)-1H-indole-6-carboxylate (2.1 g, 6.6 mmol) in THF/MeOH/H₂O (3/1/1, v/v/v, 20 mL) was added sodium hydroxide (0.66 g, 16.4 mmol). The resulting mixture was stirred at room temperature for 2 h. After the starting material disappeared, THF and methanol were removed under reduced pressure. The residue was acidified with hydrochloric acid (1N, 16.4 ml). The precipitate formed was collected through filtration and dried to afford 1-methyl-5-(2-morpholino ethoxy)-1H-indole-6-carboxylic acid as a yellow solid (750 mg, 37%). LC/MS (ES+) calcd for C₁₆H₂₀N₂O₄: 304.1; found: 305.1 [M+H]. ¹H NMR (400 MHz, DMSO-d6): δ 7.98 (s, 1H), 7.51 (d, J=1.54 Hz, 1H), 7.24 (dd, J=7.50, 0.72 Hz, 1H), 6.22 (dd, J=7.58, 1.58 Hz, 1H), 4.04 (t, J=7.08 Hz, 2H), 3.79 (d, J=0.74 Hz, 3H), 3.63 (t, J=7.11 Hz, 4H), 2.74 (t, J=7.09 Hz, 2H), 2.53 (t, J=7.11 Hz, 4H).

### Intermediate 53: 1-methyl-5-[4-(morpholin-4-yl) butoxy]-1H-indole-6-carboxylic acid

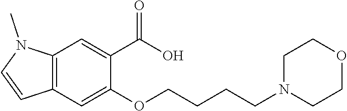 [SMILES: Cn1ccc2cc(OCCCCN3CCOCC3)c(C(=O)O)cc21]

This compound can be prepared as described above for Intermediate 52, 1-methyl-5-[2-(morpholin-4-yl)ethoxy]-1H-indole-6-carboxylic acid by substituting 2-(2-chloroethyl) morpholine with 4-(4-chlorobutyl)-morpholine (CAS No. 734495-59-1). LC/MS (ES⁺) calcd for C₁₈H₂₄N₂O₄: 332.4; found: 333.5 [M+H]. ¹H NMR (400 MHz, DMSO-d6): δ 7.97 (s, 1H), 7.67 (d, J=1.79 Hz, 1H), 7.27-7.21 (m, 1H), 6.22 (dd, J=7.56, 1.60 Hz, 1H), 4.02 (t, J=7.09 Hz, 2H), 3.79 (s, 2H), 3.59 (t, J=7.11 Hz, 4H), 2.60 (t, J=7.11 Hz, 2H), 2.46 (t, J=7.11 Hz, 4H), 1.84 (p, J=7.12 Hz, 2H), 1.58 (p, J=7.04 Hz, 2H).

### Preparation of Representative Compounds

### Example 1

### N-{3,10-dithia-5,12-diazatricyclo[7.3.0.0^{2,6}]dodeca-1,4,6,8,11-pentaen-4-yl}-3-[3-(morpholin-4-yl)propoxy]naphthalene-2-carboxamide

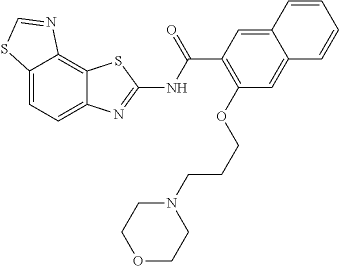 [SMILES: O=C(Nc1nc2ccc3scnc3c2s1)c1cc2ccccc2cc1OCCCN1CCOCC1]

A mixture of 3-[3-(morpholin-4-yl)ethoxy]naphthalene-2-carboxylic acid (Intermediate 34, 300 mg, 0.96 mmol), TBTU (156 mg, 0.48 mmol), and DIEA (249 mg, 1.92 mmol) in acetonitrile (6 mL) was stirred at RT for 15 min. After this time, benzo[1,2-d:3,4-d′]bis(thiazole)-2-amine (Intermediate amine 7, 240 mg, 1.1 mmol) was added in one portion at RT. The resulting mixture was stirred at RT for 12 h. The reaction mixture was diluted with DCM (10 mL) and filtered. The filter cake was purified through column chromatography (eluent: DCM:MeOH from 50:1 to 20:1) to afford the desired product (54 mg, 11%) as a white solid. LC/MS (ES⁺) calcd for C₂₅H₂₄N₄O₃S: 504.4; found: 505.2 [M+H]. ¹H NMR (400 MHz, DMSO-d6): δ 12.42 (br, 1H), 9.58 (s, 1H), 8.42 (s, 1H), 8.25 (d, J=8.64 Hz, 1H), 8.02 (d, J=8.13 Hz, 1H), 7.99-7.85 (m, 2H), 7.60 (t, J=7.60 Hz, 1H), 7.57 (s, 1H),7.46 (t, J=7.49 Hz, 1H), 4.30 (t, J=5.87 Hz, 2H), 3.58-3.42 (m, 4H), 2.54 (t, J=7.27 Hz, 2H),2.34 (br, 4H), 2.07-1.96 (m, 2H).

The following compounds in Table 3 were prepared as described above for Example 1 with the appropriate amine and carboxylic acid.

TABLE 3
Example Compounds

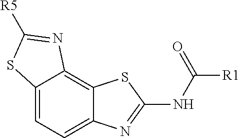

| | Amine | Acid | | | |
|---|---|---|---|---|---|
| Ex. | (Int. | (Int. | | | |
| No. | No.) | No.) | Name | R¹ | R⁵ |
| 2 | 4 | 14 | 3,5-dimethoxy-N-{11- methyl-3,10-dithia-5,12- diazatricyclo[7.3.0.0²,^{⁶}] dodeca-1,4,6,8,11- pentaen-4-yl}benzamide | 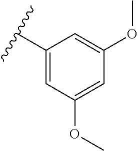 [SMILES: *c1cc(OC)cc(OC)c1] | CH₃ |
| 3 | 4 | 31 | 4-(diethyl sulfamoyl)-N- {11-methyl-3,10-dithia- 5,12- diazatricyclo[7.3.0.0²,^{⁶}] dodeca-1,4,6,8,11- pentaen-4-yl}benzamide | 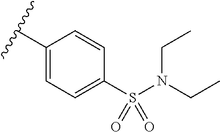 [SMILES: *c1ccc(S(=O)(=O)N(CC)CC)cc1] | CH₃ |
| 4 | 6 | 28 | N-{3,10-dithia-5,12- diazatricyclo[7.3.0.0²,^{⁶}] dodeca-1,4,6,8,11- pentaen-4-yl}-2H-1,3- benzodioxole-5- carboxamide | 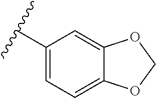 [SMILES: *c1ccc2c(c1)OCO2] | H |
| 5 | 4 | 32 | N-{11-methyl-3,10- dithia-5,12- diazatricyclo[7.3.0,0²,^{⁶}] dodeca-1,4,6,8,11- pentaen-4-yl)-4- (pentyloxy)benzamide | 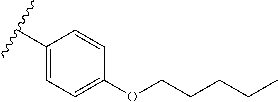 [SMILES: *c1ccc(OCCCCC)cc1] | CH₃ |
| 6 | 4 | 23 | 4-(dimethylamino)-N- {11-methyl-3,10-dithia- 5,12- diazatricyclo[7.3.0.0²,^{⁶}] dodeca-1,4,6,8,11- pentaen-4-yl}benzamide | 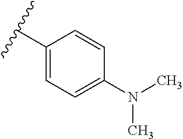 [SMILES: *c1ccc(N(C)C)cc1] | CH₃ |
| 7 | 6 | 18 | 4-chloro-N-{3,10-dithia- 5,12- diazatricyclo[7.3.0.0²,^{⁶}] dodeca-1,4,6,8,11- pentaen-4-yl}-3- (trifluoromethyl) benzamide | 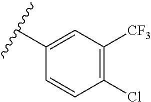 [SMILES: *c1ccc(Cl)c(C(F)(F)F)c1] | H |
| 8 | 6 | 29 | N-{3,10-dithia-5,12- diazatricyclo[7.3.0.0²,^{⁶}] dodeca-1,4,6,8,11- pentaen-4-yl}-3- (trifluoromethyl) benzamide | 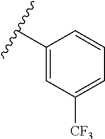 [SMILES: *c1cccc(C(F)(F)F)c1] | H |
| 9 | 6 | No | N-{3,10-dithia-5,12- diazatricyclo [7.3.0.0²,^{⁶}]dodeca- 1,4,6,8,11-pentaen-4-yl}- 3-nitrobenzamide | 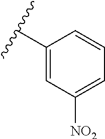 [SMILES: *c1cccc([N+](=O)[O-])c1] | H |
| 10 | 4 | 22 | N-(3-bromophenyl)-11- methyl-3,10-dithia-5,12- diazatricyclo[7.3.0.0²,^{⁶}] dodeca-1,4,6,8,11- pentaene-4-carboxamide | 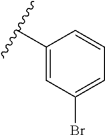 [SMILES: *c1cccc(Br)c1] | CH₃ |
| 11 | 6 | 24 | N-{3,10-dithia-5,12- diazatricyclo[7.3.0.0²,^{⁶}] dodeca-1,4,6,8,11- pentaen-4-yl}-1- benzothiophene-2- carboxamide | 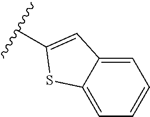 [SMILES: *c1cc2ccccc2s1] | H |
| 12 | 6 | 17 | N-{3,10-dithia-5,12- diazatricyclo[7,3.0.0²,^{⁶}] dodeca-1,4,6,8,11- pentaen-4-yl}-2,1,3- benzothiadiazole-5- carboxamide | 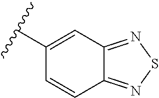 [SMILES: *c1ccc2nsnc2c1] | H |
| 13 | 6 | 33 | N-{3,10-dithia- 5,12- diazatricyclo[7.3.0.0²,^{⁶}] dodeca-1,4,6,8,11- pentaen-4-yl}-5,6,7,8- tetrahydronaphthalene-2- carboxamide | 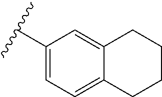 [SMILES: *c1ccc2c(c1)CCCC2] | H |
| 14 | 6 | 20 | N-{3,10-dithia-5,12- diazatricyclo[7.3.0,0²,^{⁶}] dodeca-1,4,6,8,11- pentaen-4-yl}-1- benzothiophene-5- carboxamide | 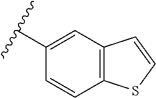 [SMILES: *c1ccc2sccc2c1] | H |
| 15 | 6 | 30 | N-{3,10-dithia-5,12- diazatricyclo[7.3.0.0²,^{⁶}] dodeca-1,4,6,8,11- pentaen-4-yl}~1- benzofuran-5- carboxamide | 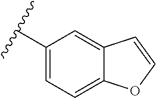 [SMILES: *c1ccc2occc2c1] | H |
| 16 | 6 | 25 | N-{3,10-dithia-5,12- diazatricyclo[7.3.0.0²,^{⁶}] dodeca-1,4,6,8,11- pentaen-4-yl}-3- methoxynaphthalene-2- carboxamide | 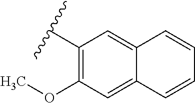 [SMILES: *c1cc2ccccc2cc1OC] | H |
| 17 | 6 | 16 | N-{3,10-dithia-5,12- diazatricyclo[7.3.0.0²,^{⁶}] dodeca-1,4,6,8,11- pentaen-4-yI}-1-methyl- 1H-indole-2- carboxamide | 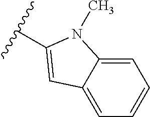 [SMILES: *c1cc2ccccc2n1C] | H |
| 18 | 3 | 27 | N-{11-ethyl-3,10-dithia- 5,12- diazatricyclo[7.3.0.0²,^{⁶}] dodeca-1,4,6,8,11- pentaen-4- yl}naphthalene-2- carboxamide | 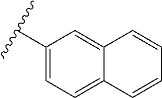 [SMILES: *c1ccc2ccccc2c1] | CH₂CH₃ |
| 19 | 1 | 27 | N-[11-(methylsulfanyl)- 3,10-dithia-5,12- diazatricyclo[7.3.0.0²,^{⁶}] dodeca-1,4,6,8,11- pentaen-4- yl]naphthalene-2- carboxamide | 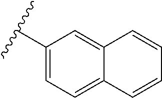 [SMILES: *c1ccc2ccccc2c1] | SCH₃ |
| 20 | 6 | 19 | N-{3,10-dithia-5,12- diazatricyclo[7.3,0.0²,^{⁶}] dodeca-1,4,6,8,11- pentaen-4-yl}-1-methyl- 1H-indole-6- carboxamide | 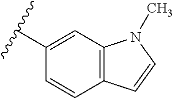 [SMILES: *c1ccc2ccn(C)c2c1] | H |
| 21 | 6 | 26 | N-{3,10-dithia-5,12- diazatricyclo[7.3.0.0²,^{⁶}] dodeca-1,4,6,8,11- pentaen-4-yl}-[1,1′- biphenyl]-4-carboxamide | 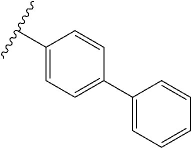 [SMILES: *c1ccc(-c2ccccc2)cc1] | H |
| 22 | 2 | 27 | N-{11-methoxy-3,10- dithia-5,12- diazatricyclo[7.3.0.0²,^{⁶}] dodeca-1,4,6,8,11- pentaen-4- yl}naphthalene-2- | 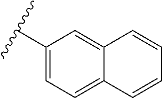 [SMILES: *c1ccc2ccccc2c1] | OCH₃ |
| | | | carboxamide | | |
| 23 | 4 | 27 | N-{11-methyl-3,10- dithia-5,12- diazatricyclo[7.3.0.0²,^{⁶}] dodeca-1,4,6,8,11- pentaen-4- yl}naphthalene-2- | 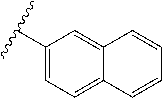 [SMILES: *c1ccc2ccccc2c1] | CH₃ |
| | | | carboxamide | | |
| 24 | 6 | 35 | N-{3,10-dithia-5,12- diazatricyclo[7.3.0.0²,^{⁶}] dodeca-1,4,6,8,11- pentaen-4-yl}-3-[2- (morpholin-4- yl)ethoxy]naphthalene-2- carboxamide | 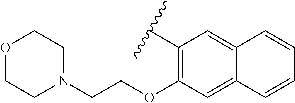 [SMILES: *c1cc2ccccc2cc1OCCN1CCOCC1] | H |
| 25 | 6 | 37 | N-{3,10-dithia-5,12- diazatricyclo[7.3.0.0²,^{⁶}] dodeca-1,4,6,8,11- pentaen-4-yl}-3-[2- (piperidin-1- yl)ethoxy]naphthalene-2- carboxamide | 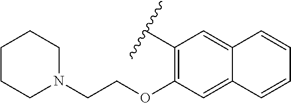 [SMILES: *c1cc2ccccc2cc1OCCN1CCCCC1] | H |
| 1 | 6 | 34 | N-{3,10-dithia-5,12- diazatricyclo[7.3.0.0²,^{⁶}] dodeca-1,4,6,8,11- pentaen-4-yl}-3-[3- (morpholin-4- yl)propoxy]naphthalene- 2-carboxamide | 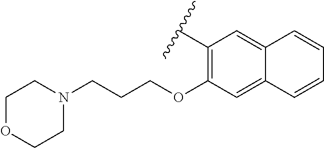 [SMILES: *c1cc2ccccc2cc1OCCCN1CCOCC1] | H |
| 27 | 6 | 38 | N-{3,10-dithia-5,12- diazatricyclo[7.3.0.0²,^{⁶}] dodeca-1,4,6,8,11- pentaen-4-yl}-3-[2- (oxan-4- yl)ethoxy]naphthalene-2- carboxamide | 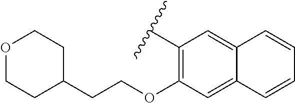 [SMILES: *c1cc2ccccc2cc1OCCC1CCOCC1] | H |
| 28 | 6 | 36 | N-{3,10-dithia-5,12- diazatricyclo[7.3.0.0²,^{⁶}] dodeca-1,4,6,8,11- pentaen-4-yl}-3-[4- (morpholin-4- yl)butoxy]naphthalene-2- carboxamide | 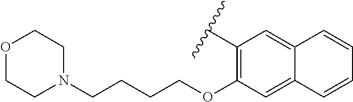 [SMILES: *c1cc2ccccc2cc1OCCCCN1CCOCC1] | H |
| 29 | 2 | 35 | N-{11-methoxy-3,10- dithia-5,12- diazatricyclo[7.3,0.0²,^{⁶}] dodeca-1,4,6,8,11- pentaen-4~yl}-3-[2- (morpholin-4- | 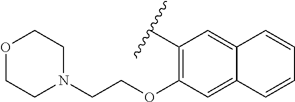 [SMILES: *c1cc2ccccc2cc1OCCN1CCOCC1] | OCH₃ |
| | | | yl)ethoxy]naphthalene-2- | | |
| | | | carboxamide | | |
| 30 | 2 | 37 | N-{11-methoxy-3,10- dithia-5,12- diazatricyclo[7.3.0.0²,^{⁶}] dodeca-1,4,6,8,11- pentaen-4-yl}-3-[2- (piperidin-1- | 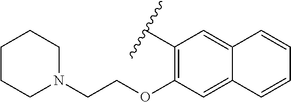 [SMILES: *c1cc2ccccc2cc1OCCN1CCCCC1] | OCH₃ |
| | | | yl)ethoxy]naphthalene-2- | | |
| | | | carboxamide | | |
| 31 | 2 | 34 | N-{11-methoxy-3,10- dithia-5,12- diazatricyclo[7.3,0.0²,^{⁶}] dodeca-1,4,6,8,11- pentaen-4-yl}-3-[3- (morpholin-4- yl)propoxy]naphthalene- 2-carboxamide | 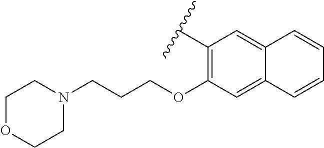 [SMILES: *c1cc2ccccc2cc1OCCCN1CCOCC1] | OCH₃ |
| 32 | 2 | 36 | N-{11-methoxy-3,10- dithia-5,12- diazatricyclo[73.0.0²,^{⁶}] dodeca-1,4,6,8,11- pentaen-4-yl}-3-[4- (morpholin-4- | 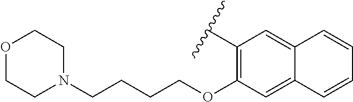 [SMILES: *c1cc2ccccc2cc1OCCCCN1CCOCC1] | OCH₃ |
| | | | yl)butoxy]naphthalene-2- | | |
| | | | carboxamide | | |
| 33 | 6 | 40 | N-{3,10-dithia-5,12- diazatricyclo[7.3.0.0²,^{⁶}] dodeca-1,4,6,8,11- pentaen-4-yl}-3-(2-{2- oxa-5- azabicyclo[2.2.1]heptan- 5-yl}ethoxy)naphthalene- 2-carboxamide | 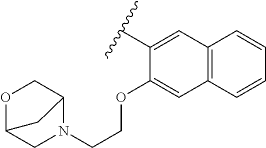 [SMILES: *c1cc2ccccc2cc1OCCN1CC2CC1CO2] | H |
| 34 | 6 | 42 | N-{3,10-dithia-5,12- diazatricyclo[7.3.0.0²,^{⁶}] dodeca-1,4,6,8,11- pentaen-4-yl}-3-(2-{2- oxa-6- azaspiro[3.3]heptan-6- yl}ethoxy)naphthalene-2- carboxamide | 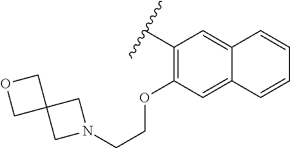 [SMILES: *c1cc2ccccc2cc1OCCN1CC2(COC2)C1] | H |
| 35 | 1 | 35 | N-[11-(methylsulfanyl)- 3,10-dithia-5,12- diazatricyclo[7.3.0.0²,^{⁶}] dodeca-1,4,6,8,11- pentaen-4-yl]-3-[2- (morpholin-4- | 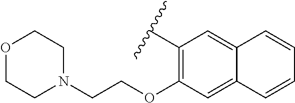 [SMILES: *c1cc2ccccc2cc1OCCN1CCOCC1] | SCH₃ |
| | | | yl)ethoxy]naphthalene-2- | | |
| | | | carboxamide | | |
| 36 | 1 | 34 | N-[11-(methylsulfanyl)- 3,10-dithia-5,12- diazatricyclo[7.3.0.0²,^{⁶}] dodeca-1,4,6,8,11- pentaen-4-yl]-3-[3- (molpholin-4- yl)propoxy]naphthalene- 2-carboxamide | 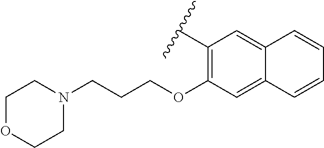 [SMILES: *c1cc2ccccc2cc1OCCCN1CCOCC1] | SCH₃ |
| 37 | 1 | 36 | N-[11-(methylsulfanyl)- 3,10-dithia-5,12- diazatricyclo[7.3.0.0²,^{⁶}] dodeca-1,4,6,8,11- pentaen-4-yl]-3-[4- (morpholin-4- | 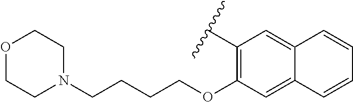 [SMILES: *c1cc2ccccc2cc1OCCCCN1CCOCC1] | SCH₃ |
| | | | yl)butoxy]naphthalene-2- | | |
| | | | carboxamide | | |
| 38 | 6 | 46 | N-{3,10-dithia-5,12- diazatricyclo[7.3.0.0²,^{⁶}] dodeca-1,4,6,8,11- pentaen-4-yl}-3-[4- (morpholin-4-yl)butoxy]- [1,1′-biphenyl]-4- carboxamide | 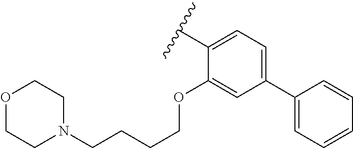 [SMILES: *c1ccc(-c2ccccc2)cc1OCCCCN1CCOCC1] | H |
| 39 | 6 | 47 | N-{3,10-dithia-5,12- diazatricyclo[7.3.0.0²,^{⁶}] dodeca-1,4,6,8,11- pentaen-4-yl}-6-[4- (morpholin-4- yl)butoxy]naphthalene-2- carboxamide | 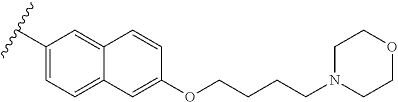 [SMILES: *c1ccc2cc(OCCCCN3CCOCC3)ccc2c1] | H |
| 40 | 6 | 48 | N-{3,10-dithia-5,12- diazatricyclo[7.3.0.0²,^{⁶}] dodeca-1,4,6,8,11- pentaen-4-yl}-6-[2- (morpholin-4-yl)ethoxy]- 2H-1,3-benzodioxole-5- carboxamide | 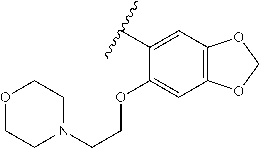 [SMILES: *c1cc2c(cc1OCCN1CCOCC1)OCO2] | H |
| 41 | 6 | 49 | N-{3,10-dithia-5,12- diazatricyclo[7.3.0.0²,^{⁶}] dodeca-1,4,6,8,11- pentaen-4-yl}-6-[4- (morpholin-4-yl)butoxy]- 2H-1,3-benzodioxole-5- carboxamide | 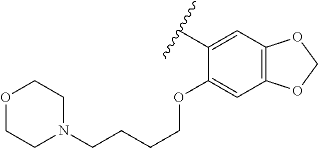 [SMILES: *c1cc2c(cc1OCCCCN1CCOCC1)OCO2] | H |
| 42 | 6 | 50 | N-{3,10-dithia-5,12- diazatricyclo[7.3.0.0²,^{⁶}] dodeca-1,4,6,8,11- pentaen-4-yl}-6-[2- (morpholin-4-yl)ethoxy]- 1-benzothiophene-5- carboxamide | 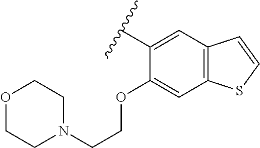 [SMILES: *c1cc2ccsc2cc1OCCN1CCOCC1] | H |
| 43 | 6 | 51 | N-{3,10-dithia-5,12- diazatricyclo[7.3,0.0²,^{⁶}] dodeca-1,4,6,8,11- pentaen-4-yl}-6-[4- (morpholin-4-yl)butoxy]- 1-benzothiophene-5- carboxamide | 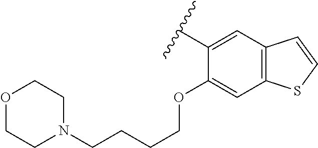 [SMILES: *c1cc2ccsc2cc1OCCCCN1CCOCC1] | H |

NMR and LC/MS mass spectrometry data for the compounds of Table 3 are provided below in Table 4.

TABLE 4
¹H NMR and LC/MS Data for Representative Compounds

| | | LC/MS |
|---|---|---|
| Ex. No. | ¹H NMR | (MH⁺) |
| 1 | 1H NMR (400 MHz, CDCl₃): δ 12.43 (s, 1H), 9.58 (s, 1H), | 505.6 |
| | 8.42 (s, 1H), 8.25 (d, J = 8.6 Hz, 1H), 8.02 (d, J = 8.2 Hz, 1H), | |
| | 7.91 (dd, J = 8.6, 2.6 Hz, 2H), 7.66-7.53 (m, 2H), 7.46 (t, J = 7.5 Hz, | |
| | 1H), 4.30 (t, J = 6.1 Hz, 2H), 3.49 (t, J = 4.6 Hz, 4H), 2.55 (d, J = 7.2 Hz, | |
| | 2H), 2.34 (t, J = 4.6 Hz, 4H), 2.02 (p, J = 6.6 Hz, 2H) | |
| 2 | ¹H NMR (400 MHz, DMSO-d6): δ 8.00 (d, J = 7.50 Hz, 1H), | 386.5 |
| | 7.78 (d, J = 7.50 Hz, 1H), 7.13 (d, J = 1.49 Hz, 2H), 6.65 (t, J = 1.46 Hz, | |
| | 1H), 3.84 (s, 4H), 2.82 (s, 2H) | |
| 3 | ¹H NMR (400 MHz, DMSO-d6): δ 8.25-8.19 (m, 2H), 8.00 (d, | 461.6 |
| | J = 7.50 Hz, 1H), 7.86-7.81 (m, 2H), 7.78 (d, J = 7.50 Hz, 1H), | |
| | 3.22 (q, J = 7.97 Hz, 4H), 2.82 (s, 2H), 1.11 (t, J = 7.97 Hz, 6H) | |
| 4 | ¹H NMR (400 MHz, CDCl₃): δ 9.34 (s, 1H), 9.24 (s, 1H), | 356.4 |
| | 7.90-7.82 (m, 2H), 7.47-7.37 (m, 2H), 6.96 (d, J = 8.4 Hz, 1H), | |
| | 6.04 (d, J = 2.4 Hz, 1H), 5.99 (d, J = 2.4 Hz, 1H) | |
| 5 | ¹H NMR (400 MHz, DMSO-d6): δ 8.13-8.07 (m, 2H), 8.00 (d, J = 7.51 Hz, | 412.5 |
| | 1H), 7.78 (d, J = 7.51 Hz, 1H), 7.01-6.95 (m, 2H), | |
| | 3.87 (t, J = 7.11 Hz, 2H), 2.82 (s, 2H), 1.83-1.74 (m, 2H), | |
| | 1.46-1.34 (m, 4H), 0.95-0.87 (m, 3H) | |
| 6 | ¹H NMR (400 MHz, CDCl₃): δ 8.99 (s, 1H), 7.74 (d, J = 1.4 Hz, | 369.5 |
| | 1H), 7.51-7.44 (m, 1H), 6.73-6.67 (m, 1H), 3.03 (s, 2H), | |
| | 2.86 (s, 1H) | |
| 7 | ¹H NMR (400 MHz, CDCl₃): δ 9.30 (s, 1H), 9.25 (s, 1H), 7.94 (d, | 414.8 |
| | J = 1.9 Hz, 1H), 7.83-7.74 (m, 2H), 7.68 (d, J = 8.4 Hz, 1H), | |
| | 7.56 (d, J = 8.4 Hz, 1H) | |
| 8 | ¹H NMR (400 MHz, CDCl₃): δ 9.35 (s, 1H), 9.25 (s, 1H), 8.24 (t, | 380.4 |
| | J = 2.2 Hz, 1H), 7.93 (ddd, J = 7.5, 2.2, 1.5 Hz, 1H), | |
| | 7.80-7.69 (m, 2H), 7.73-7.65 (m, 3H) | |
| 9 | ¹H NMR (400 MHz, CDCl₃): δ 9.57 (s, 1H), 9.25 (s, 1H), 8.94 (t, | 357.4 |
| | J = 2.3 Hz, 1H), 8.50-8.42 (m, 2H), 7.82-7.74 (m, 2H), 7.68 (d, | |
| | J = 8.4 Hz, 1H) | |
| 10 | ¹H NMR (400 MHz, CDCl₃): δ 9.28 (s, 1H), 8.09-8.03 (m, 1H), | 405.3 |
| | 7.91-7.81 (m, 3H), 7.69 (ddd, J = 8.0, 2.1, 1.2 Hz, 1H), | |
| | 7.63-7.56 (m, 1H), 7.50 (t, J = 2.4 Hz, 1H), 7.50-7.39 (m, 2H), | |
| | 3.05-2.93 (m, 4H) | |
| 11 | ¹H NMR (400 MHz, CDCl₃): δ 9.63 (s, 1H), 9.35 (s, 1H), 8.42 (d, | 368.5 |
| | J = 2.1 Hz, 1H), 7.90-7.76 (m, 5H), 7.37-7.29 (m, 2H) | |
| 12 | ¹H NMR (400 MHz, CDCl₃): δ 9.34 (s, 1H), 8.71 (d, J = 1.9 Hz, | 370.4 |
| | 1H), 8.26 (d, J = 8.4 Hz, 1H), 7.94 (dd, J = 8.4, 2.0 Hz, 1H), | |
| | 7.86 (d, J = 2.2 Hz, 2H) | |
| 13 | ¹H NMR (400 MHz, DMSO-d6): δ 8.00 (d, J = 7.51 Hz, 1H), | 366.5 |
| | 7.78 (d, J = 7.51 Hz, 1H), 7.72-7.64 (m, 2H), 7.12 (dt, J = 7.56, 1.06 Hz, | |
| | 1H), 2.84-2.72 (m, 6H), 1.74 (tdd, J = 7.17, 3.85, 2.00 Hz, | |
| | 4H) | |
| 14 | ¹H NMR (400 MHz, CDCl₃): δ 9.58 (s, 2H), 9.35 (s, 2H), | 368.5 |
| | 8.32 (dt, J = 2.2, 1.2 Hz, 2H), 7.95-7.86 (m, 3H), 7.89-7.82 (m, 6H), | |
| | 7.82 (s, 1H), 7.47-7.39 (m, 5H) | |
| 15 | ¹H NMR (400 MHz, CDCl₃): δ 9.66 (s, 1H), 9.35 (s, 1H), | 352.4 |
| | 8.16 (dd, J = 2.2, 1.4 Hz, 1H), 7.99 (d, J = 1.8 Hz, 1H), 7.90-7.82 (m, | |
| | 3H), 7.64 (d, J = 8.5 Hz, 1H), 7.08 (t, J = 2.1 Hz, 1H) | |
| 16 | ¹H NMR (400 MHz, CDCl₃): δ 9.62 (s, 1H), 9.28 (s, 1H), 8.38 (d, | 392.5 |
| | J = 1.8 Hz, 1H), 7.89-7.81 (m, 4H), 7.66 (dt, J = 7.8, 2.1 Hz, | |
| | 1H), 7.48-7.39 (m, 4H), 3.85 (s, 3H) | |
| 17 | ¹H NMR (400 MHz, CDCl₃): δ 9.35 (s, 1H), 7.91 (d, J = 8.4 Hz, | 365.4 |
| | 1H), 7.85 (d, J = 8.4 Hz, 1H), 7.70-7.64 (m, 1H), 7.42-7.36 (m, | |
| | 1H), 7.34-7.26 (m, 2H), 7.19 (td, J = 7.8, 1.5 Hz, 1H), 3.87 (s, | |
| | 2H) | |
| 18 | 1H NMR (500 MHz, DMSO-d6): δ 9.22 (s, 1H), 7.92 (d, J = 1.62 Hz, | 390.5 |
| | 1H), 7.83-7.76 (m, 3H), 7.51-7.46 (m, 1H), 7.28 (dd, J = 7.42, | |
| | 0.92 Hz, 1H), 6.51 (dd, J = 7.57, 1.44 Hz, 1H), 3.80 (s, 2H) | |
| 19 | ¹H NMR (400 MHz, DMSO-d6): 8.35 (t, J = 1.58 Hz, 1H), | 408.5 |
| | 8.13-8.04 (m, 2H), 7.94 (ddd, J = 7.78, 3.78, 1.52 Hz, 2H), 7.87 (d, J = 7.50 Hz, | |
| | 1H), 7.81 (dd, J = 7.96, 1.52 Hz, 1H), 7.65-7.57 (m, | |
| | 2H), 2.80 (s, 2H) | |
| 20 | 1H NMR (500 MHz, DMSO-d6): δ 8.35 (t, J = 1.46 Hz, 1H), | 365.4 |
| | 8.09 (ddd, J = 5.70, 2.76, 1.44 Hz, 1H), 8.02 (d, J = 7.51 Hz, 1H), | |
| | 7.94 (dq, J = 5.96, 1.70 Hz, 2H), 7.84-7.77 (m, 2H), 7.65-7.57 (m, | |
| | 2H), 2.96 (q, J = 8.02 Hz, 2H), 1.31 (t, J = 7.98 Hz, 3H) | |
| 21 | ¹H NMR (400 MHz, CDCl₃): δ 9.34 (s, 1H), 9.27 (s, 1H), | 388.5 |
| | 8.13-8.06 (m, 2H), 7.89-7.81 (m, 2H), 7.71-7.65 (m, 2H), | |
| | 7.62-7.55 (m, 2H), 7.48-7.40 (m, 2H), 7.40-7.32 (m, 1H) | |
| 22 | ¹H NMR (400 MHz, CDCl₃): δ 9.46 (s, 1H), 8.39 (t, J = 2.0 Hz, | 392.5 |
| | 1H), 8.01-7.92 (m, 5H), 7.89 (d, J = 8.5 Hz, 1H), 7.58-7.49 (m, | |
| | 4H), 3.99 (s, 3H) | |
| 23 | ¹H NMR (400 MHz, CDCl₃): δ 9.48 (s, 1H), 8.39 (t, J = 1.8 Hz, | 376.5 |
| | 0H), 8.01-7.92 (m, 2H), 7.87 (dd, J = 26.7, 8.5 Hz, 1H), | |
| | 7.57-7.49 (m, 1 H), 2.86 (s, 1H) | |
| 24 | ¹H NMR (400 MHz, CDCl₃): δ 9.69 (s, 1H), 9.27 (s, 1H), | 491.6 |
| | 8.12-8.08 (m, 1H), 7.91-7.81 (m, 3H), 7.69-7.63 (m, 1H), | |
| | 7.48-7.39 (m, 2H), 7.36 (d, J = 1.7 Hz, 1H), 4.41 (t, J = 6.5 Hz, 2H), | |
| | 3.69 (t, J = 6.0 Hz, 4H), 2.70 (t, J = 6.5 Hz, 2H), 2.59-2.44 (m, | |
| | 4H) | |
| 25 | ¹H NMR (400 MHz, DMSO-d6): δ 9.23 (s, 1H), 8.46 (d, J = 1.41 Hz, | |
| | 1H), 7.93 (dt, J = 6.92, 1.68 Hz, 1H), 7.80 (s, 2H), 7.76 (dt, J = 7.06, | |
| | 1.89 Hz, 1H), 7.54-7.44 (m, 3H), 4.12 (t, J = 7.09 Hz, | |
| | 2H), 2.97 (t, J = 7.09 Hz, 2H), 2.51 (t, J = 7.04 Hz, 4H), 1.54 (pd, | |
| | J = 7.03, 0.86 Hz, 4H), 1.47-1.38 (m, 2H) | |
| 27 | 1H NMR (400 MHz, CDCl₃): δ 12.45 (s, 1H), 9.58 (s, 1H), | 490.6 |
| | 8.40 (s, 1H), 8.25 (d, J = 8.6 Hz, 1H), 8.01 (d, J = 8.2 Hz, 1H), | |
| | 7.90 (dd, J = 8.4, 2.9 Hz, 2H), 7.63-7.57 (m, 2H), 7.45 (t, J = 7.5 Hz, | |
| | 1H), 4.30 (t, J = 6.0 Hz, 2H), 3.78-3.70 (m, 2H), 3.27-3.17 (m, | |
| | 3H), 1.79 (q, J = 6.3 Hz, 3H), 1.67 (d, J = 13.0 Hz, 3H), 1.18 (dd, | |
| | J = 12.2, 4.4 Hz, 2H) | |
| 28 | 1H NMR (400 MHz, DMSO-d6): δ 12.52 (br, 1H), 9.58 (s, 1H), | 519.7 |
| | 8.38 (s, 1H), 8.25 (d, J = 8.4 Hz, 1H), 8.00 (d, J = 8.4 Hz, 1H), | |
| | 7.91 (dd, J = 8.0, 6.0 Hz, 2H), 7.59 (t, J = 7.6 Hz, 1H), 7.56 (s, | |
| | 1H), 7.45 (t, J = 7.6 Hz, 1H), 4.29-4.23 (m, 2H), 3.41-3.34 (m, | |
| | 4H), 2.34-2.25 (m, 2H), 2.25-2.15 (m, 4H), 1.90-1.80 (m, 2H), | |
| | 1.70-1.61 (m, 2H) | |
| 29 | ¹H NMR (400 MHz, DMSO-d6): δ 12.71 (s, 1H), 11.04 (s, 1H), | 521.6 |
| | 8.33 (s, 1H), 7.97 (dd, 3H, J = 15.1, 8.6 Hz), 7.92 (s, 1H), 7.64 (s, | |
| | 1H), 7.50 (s, 1H), 7.16 (t, 1H, J = 7.5 Hz), 4.64 (s, 2H), 4.24 (s, | |
| | 3H), 3.90-3.64 (m, 8H), 3.17 (s, 2H) | |
| 30 | 1H NMR (400 MHz, DMSO-d6): δ 12.71 (s, 1H), 10.06 (s, 1H), | 519.6 |
| | 8.36 (br, 1H), 8.05-7.90 (m, 3H), 7.74-7.69 (m, 1H), 7.66-7.58 (m, | |
| | 2H), 7.51-7.46 (m, 1H), 4.63-4.53 (m, 2H), 4.26 (s, 3H), | |
| | 3.60-3.47 (m, 2H), 3.20-2.80 (m, 4H), 1.80-1.56 (m, 6H) | |
| 31 | ¹H NMR (400 MHz, DMSO-d6): δ 12.48 (s, 1H), 10.75 (s, 1H), | 535.6 |
| | 8.39 (s, 1H), 8.04 (d, 1H, J = 8.1 Hz), 7.99 (d, 1H, J = 8.6 Hz), | |
| | 7.91 (d, 1H, J = 8.2 Hz), 7.74 (d, 1H, J = 8.5 Hz), 7.64 (d, 1H, J = 8.5 Hz), | |
| | 7.57 (d, 1H, J = 8.5 H)z, 7.50 (d, 1H, J = 8.5 Hz), 4.34 (s, | |
| | 2H), 4.26 (s, 3H), 3.95 (d, 2H, J = 12.2 Hz), 3.78 (s, 2H), 3.53 (d, | |
| | 2H, J = 12.4 Hz), 3.41 (d, 2H, J = 12.4 Hz), 3.07 (d, 2H, J = 10.6 Hz), | |
| | 2.30 (s, 2H) | |
| 32 | 1H NMR (400 MHz, DMSO-d6): δ 12.47 (br, 1H), 8.37 (s, 1H), | 549.7 |
| | 7.99 (dd, J = 8.4, 11.6 Hz, 2H), 7.90 (d, J = 8.0 Hz, 1H), 7.72 (d, J = 8.8 Hz, | |
| | 1H), 7.60 (t, J = 7.4 Hz, 1H), 7.55 (s, 1H), 7.45 (d, J = 7.6 Hz, | |
| | 1H), 4.31-4.23 (m, 5H), 3.46-3.37 (m, 4H), 2.36-2.18 (m, | |
| | 6H), 1.90-1.81 (m, 2H), 1.73-1.62 (m, 2H) | |
| 33 | ¹H NMR (400 MHz, DMSO-d6): δ 12.94 (br, 1H), 9.59 (s, 1H), | 503.6 |
| | 8.36-8.22 (m, 2H), 8.06-7.90 (m, 3H), 7.67-7.45 (m, 3H), | |
| | 4.74-4.55 (m, 4H), 4.52-4.40 (m, 2H), 4.36-4.27 (m, 2H), 3.26-3.00 (m, | |
| | 2H), 2.05-1.65 (m, 2H) | |
| 34 | 1H NMR (400 MHz, DMSO-d6): δ 9.58 (s, 1H), 8.54 (s, 1H), | 503.6 |
| | 8.25 (d, J = 8.8 Hz, 1H), 8.05 (d, J = 8.4 Hz, 1H), 7.98 (d, J = 8.8 Hz, | |
| | 1H), 7.89 (d, J = 8.0 Hz, 1H), 7.65-7.59 (m, 2H), 7.47 (t, J = 7.4 Hz, | |
| | 1H), 4.49-4.45 (m, 4H), 4.35-4.29 (m, 2H), 3.56-3.51 (m, | |
| | 4H), 2.90-2.85 (m, 2H) | |
| 35 | ¹H NMR (400 MHz, DMSO-d6): δ 12.80 (br, 1H), 11.81 (s, 1H), | 537.7 |
| | 8.30 (s, 1H), 8.10 (d, J = 8.4 Hz, 1H), 8.01 (d, J = 8.4 Hz, 1H), | |
| | 7.94 (d, J = 8.0 Hz, 1H), 7.79 (d, J = 8.8 Hz, 1H), 7.64-7.59 (m, | |
| | 2H), 7.51-7.45 (m, 1H), 4.72-4.67 (m, 2H), 3.98-3.85 (m, 4H), | |
| | 3.66-3.58 (m, 4H), 3.20-3.08 (m, 2H), 2.87 (s, 3H) | |
| 36 | ¹H NMR (400 MHz, DMSO-d6): δ 12.53 (s, 1H), 11.20-11.10 (m, | 551.7 |
| | 1H), 8.39 (s, 1H), 8.10 (d, J = 8.8 Hz, 1H), 8.03 (d, J = 8.4 Hz, | |
| | 1H), 7.91 (d, J = 8.0 Hz, 1H), 7.81 (d, J = 8.4 Hz, 1H), 7.61 (t, J = 7.6 Hz, | |
| | 1H), 7.56 (s, 1H), 7.46 (t, J = 7.4 Hz, 1H), 4.37-4.32 (m, | |
| | 2H), 3.96-3.90 (m, 2H), 3.83-3.75 (m, 2H), 3.55-3.49 (m, 2H), | |
| | 3.43-3.36 (m, 2H), 3.11-3.02 (m, 2H), 2.87 (s, 3H), 2.36-2.28 (m, | |
| | 2H) | |
| 37 | ¹H NMR (400 MHz, DMSO-d6): δ 12.60 (s, 1H), 10.52 (s, 1H), | 551.7 |
| | 8.37 (s, 1H), 8.12 (d, J = 8.8 Hz, 1H), 8.03 (d, J = 8.4 Hz, 1H), | |
| | 7.92 (d, J = 8.0 Hz, 1H), 7.81 (d, J = 8.4 Hz, 1H), 7.62 (t, J = 7.6 Hz, | |
| | 1H), 7.56 (s, 1H), 7.43 (t, J = 7.4 Hz, 1H), 4.28 (s, 2H), | |
| | 3.86-3.84 (m, 2H), 3.68-3.63 (m, 2H), 3.39-3.20 (m, 2H), 3.10-3.03 (m, | |
| | 2H), 3.00-2.9 (m, 2H), 2.87 (s, 3H), 1.92-1.90 (m, 4H) | |
| 38 | 1H NMR (400 MHz, DMSO-d6): δ 12.23 (s, 1H), 10.40 (br, 1H), | 545.7 |
| | 9.58 (s, 1H), 8.26 (d, J = 8.4 Hz, 1H), 7.91 (dd, J = 4.6, 8.0 Hz, | |
| | 2H), 7.80 (d, J = 7.6 Hz, 2H), 7.53 (t, J = 7.4 Hz, 2H), | |
| | 7.50-7.42 (m, 3H), 4.40-4.34 (m, 2H), 3.80-3.50 (m, 4H), 3.27-2.80 (m, 4H), | |
| | 2.43-2.15 (m, 2H), 1.96-1.84 (m, 4H) | |
| 39 | ¹H NMR (400 MHz, DMSO-d6): δ 13.08 (br, 1H), 9.58 (s, 1H), | 519.7 |
| | 8.81 (s, 1H), 8.25 (d, J = 8.4 Hz, 1H), 8.16 (d, J = 7.6 Hz, 1H), | |
| | 8.06-7.99 (m, 1H), 7.99-7.90 (m, 2H), 7.49-7.40 (m, 1H), | |
| | 7.34-7.25 (m, 1H), 4.25-4.12 (m, 2H), 3.88-3.62 (m, 4H), 3.20-2.60 (m, | |
| | 5H), 2.02-1.70 (m, 5H) | |
| 40 | ¹H NMR (400 MHz, DMSO-d6): δ 12.31 (br, 1H), 10.81 (br, 1H), | 485.6 |
| | 9.57 (s, 1H), 8.23 (d, J = 8.4 Hz, 1H), 7.93-7.84 (m, 1H), | |
| | 7.35-7.25 (m, 1H), 7.10 (s, 1H), 6.15 (s, 2H), 4.58-4.48 (m, 2H), | |
| | 4.02-3.72 (m, 4H), 3.68-3.53 (m, 4H), 3.28-3.12 (m, 2H) | |
| 41 | ¹H NMR (400 MHz, DMSO-d6): δ 11.92 (s, 1H), 10.63 (br, 1H), | 513.6 |
| | 9.57 (s, 1H), 8.24 (d, J = 9.2 Hz, 1H), 7.91 (d, J = 8.4 Hz, 1H), | |
| | 7.39 (s, 1H), 7.07 (s, 1H), 6.14 (s, 2H), 4.26-4.20 (m, 2H), | |
| | 3.90-3.82 (m, 2H), 3.76-3.66 (m, 2H), 3.43-3.35 (m, 2H), 3.25-3.15 (m, | |
| | 2H), 3.09-2.97 (m, 2H), 2.01-1.85 (m, 4H) | |
| 42 | ¹H NMR (400 MHz, DMSO-d6): δ 12.27 (s, 1H), 9.58 (s, 1H), | 497.6 |
| | 8.51 (s, 1H), 8.24 (d, J = 8.8 Hz, 1H), 7.97 (s, 1H), 7.88 (d, J = 8.8 Hz, | |
| | 1H), 7.71 (d, J = 5.2 Hz, 1H), 7.54 (d, J = 5.6 Hz, 1H), | |
| | 4.47-4.41 (m, 2H), 3.65-3.57 (m, 4H), 2.89-2.83 (m, 2H), 2.60-2.52 (m, | |
| | 4H) | |
| 43 | ¹H NMR (400 MHz, DMSO-d6): δ 12.34 (s, 1H), 9.58 (s, 1H), | 525.7 |
| | 8.31 (s, 1H), 8.25 (d, J = 8.8 Hz, 1H), 7.91 (d, J = 8.4 Hz, 1H), | |
| | 7.89 (s, 1H), 7.69 (d, J = 5.6 Hz, 1H), 7.49 (d, J = 5.6 Hz, 1H), | |
| | 4.27-4.22 (m, 2H), 3.42-3.36 (m, 4H), 2.35-2.28 (m, 2H), | |
| | 2.27-2.18 (m, 4H), 1.89-1.81 (m, 2H), 1.71-1.62 (m, 2H) | |

### Example 44

### [N-(7-hydroxybenzo[1,2-d:3,4-d′]bis(thiazole)-2-yl)-3-(4-morpholinobutoxy)-2-naphthamide hydrochloride]

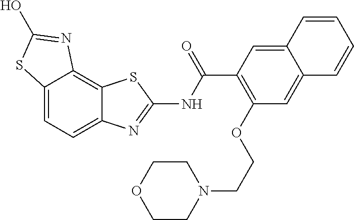 [SMILES: O=C(Nc1nc2ccc3sc(O)nc3c2s1)c1cc2ccccc2cc1OCCN1CCOCC1]

To a suspension of N-(7-methoxybenzo[1,2-d:3,4-d′]bis(thiazole)-2-yl)-3-(4-morpholinobutoxy)-2-naphthamide (Example 32, 300 mg, 0.55 mmol) in DCM (10 mL) and methanol (10 mL) was added a solution of hydrogen chloride in methanol (4M, 20 mL). The resulting mixture was heated to refluxing temperature and monitored with LC/MS until starting material was consumed. Upon completion, the reaction mixture was concentrated under reduced pressure. The concentrate was triturated with ether and dried to afford the title compound as a yellow solid (306 mg, 98%). LC/MS (ES⁺): m/z calculated for C₂₇H₂₆N₄O₄S₂: 534.1; found: 535.1 [M+H]. ¹H NMR (400 MHz, DMSO-d₆): δ 12.57 (s, 1H), 12.53 (br, 1H), 10.77 (br, 1H), 8.34 (s, 1H), 8.02 (d, J=8.0 Hz, 1H), 7.92 (d, J=8.4 Hz, 1H), 7.69 (d, J=8.4 Hz, 1H), 7.62-7.56 (m, 3H), 7.46 (t, J=7.6 Hz, 1H), 4.30-4.24 (m, 2H), 3.92-3.83 (m, 2H), 3.77-3.71 (m, 2H), 3.37-3.34 (m, 2H), 3.20-3.14 (m, 2H), 3.05-2.94 (m, 2H), 2.00-1.85 (m, 4H)

### Example 45

### N-{3,10-Dithia-5,12-diazatricyclo[7.3.0.0^{2,6}]dodeca-1,4,6,8,11-pentaen-4-yl}-3-{2-[ethyl(2-hydroxyethyl)amino]ethoxy}naphthalene-2-carboxamide carboxamide

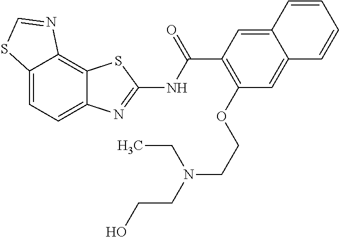 [SMILES: CCN(CCO)CCOc1cc2ccccc2cc1C(=O)Nc1nc2ccc3scnc3c2s1]

This compound can be prepared as described above for Example 1, starting with 2-[ethyl(2-hydroxyethyl)amino]ethan-1-ol in place of 2-aminoethanol. LC/MS (ES⁺) calcd for C₂₅H₂₄N₄O₃S₂: 492.1; found: 493.1 [M+H]. ¹H NMR (400 MHz, CDCl₃): δ 9.60 (s, 1H), 9.27 (s, 1H), 8.32 (d, J=1.8 Hz, 1H), 7.91-7.81 (m, 3H), 7.66 (ddd, J=6.3, 2.6, 1.6 Hz, 1H), 7.48-7.39 (m, 3H), 4.34 (t, J=6.5 Hz, 2H), 3.79 (dd, J=7.7, 6.8 Hz, 1H), 3.67 (q, J=6.9 Hz, 2H), 2.88 (t, J=6.5 Hz, 2H), 2.77 (t, J=6.8 Hz, 2H), 2.67 (q, J=7.2 Hz, 2H), 1.06 (t, J=7.2 Hz, 3H).

### Example 46

### N-{3,10-dithia-5,12-diazatricyclo[7.3.0.0²,⁶]dodeca-1,4,6,8,11-pentaen-4-yl}-3-{2-[(2-hydroxyethyl)amino]ethoxy}naphthalene-2-carboxamide

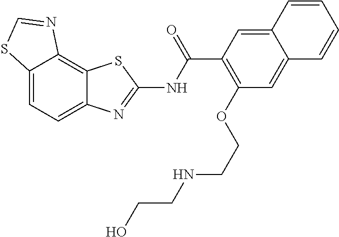 [SMILES: O=C(Nc1nc2ccc3scnc3c2s1)c1cc2ccccc2cc1OCCNCCO]

This compound can be prepared as described above for Example 1, starting with (2-hydroxyethyl)amino]ethan-1-ol in place of 2-aminoethanol. LC/MS (ES⁺) calcd for C₂₃H₂₀N₄O₃S₂: 464.5; found: 465.5 [M+H]. ¹H NMR (400 MHz, DMSO-d6) δ 9.54 (s, 1H), 8.48 (s, 1H), 8.18 (d, J=8.8 Hz, 1H), 8.03 (d, J=8.0 Hz, 1H), 7.87 (dd, J=12.0, 8.8 Hz, 2H), 7.68 (s, 1H), 7.59 (t, J=7.6 Hz, 1H), 7.46 (t, J=7.6 Hz, 1H), 4.56-4.46 (m, 2H), 3.62-3.53 (m, 2H), 3.17-3.07 (m, 2H), 2.94-2.86 (m, 2H).

### Example 47

### N-{3,10-Dithia-5,12-diazatricyclo[7.3.0.0^{2,6}]dodeca-1,4,6,8,11-pentaen-4-yl}-3-[2-(piperazin-1-yl)ethoxy]naphthalene-2-carboxamide

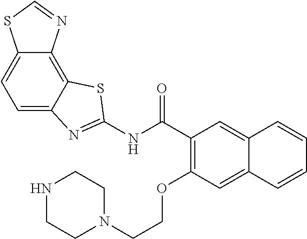 [SMILES: O=C(Nc1nc2ccc3scnc3c2s1)c1cc2ccccc2cc1OCCN1CCNCC1]

Step 1: A mixture of 3-(2-(4-(tert-butoxycarbonyl)piperazin-1-yl)ethoxy)-2-naphthoic acid (Intermediate 45, 500 mg, 1.25 mmol), TBTU (200 mg, 0.63 mmol), and DIEA (322 mg, 2.5 mmol) in acetonitrile (10 mL) was stirred at RT for 15 min, and then benzo[1,2-d:3,4-d′] bis(thiazole)-2-amine (259 mg, 1.25 mmol) was added in one portion at RT. The resulting mixture was stirred at RT for 12 h. The reaction mixture was diluted with DCM (10 mL) and filtered. The filtered cake was purified by silica gel column chromatography (eluent: DCM:MeOH from 100:1 to 50:1) to afford the desired product (130 mg, 18%) as white solid. LC/MS (ES⁺) calcd for C₃₀H₃₁N₅O₄S₂: 589.3; found: 590.3 [M+H]. ¹H NMR (400 MHz, CDCl₃): δ 11.87 (br, 1H),9.14 (s, 1H), 8.94 (s, 1H), 7.97 (s, 1H), 7.95 (s, 1H), 7.84 (d, J=8.60 Hz, 1H), 7.76 (d, J=8.25 Hz, 1H), 7.60-7.56 (m, 1H), 7.47-7.43 (m, 1H), 7.31 (s, 1H), 4.47 (t, J=5.09 Hz, 2H), 3.60 (br, 4H), 3.06 (t, J=5.09 Hz, 2H), 2.67 (br, 4H), 1.43 (s, 9H).

Step 2: To a mixture of tert-butyl 4-(2-((3-(benzo[1,2-d:3,4-d′]bis(thiazole)-2-ylcarbamoyl) naphthalen-2-yl)oxy)ethyl)piperazine-1-carboxylate (Int. Acid No., 130 mg, 0.22 mmol) in DCM (4 mL) was added TFA (1 mL), and the resulting mixture was stirred at RT for 2 h. The reaction mixture was treated with aqueous NaHCO₃ solution to pH 8 and extracted with DCM/MeOH (4:1, 3×5 mL). The combined organic layers were dried over Na₂SO₄ and concentrated in vacuo to give a crude product that was purified by silica gel column chromatography (eluent: DCM:MeOH:TEA=20:1:0.4) to afford the title compound (60 mg, 56%) as a white solid. LC/MS (ES⁺) calcd for C₂₅H₂₃N₅O₂S₂: 489.3; found: 490.5 [M+H]. ¹H NMR (400 MHz, DMSO-d₆): δ 9.57 (s, 1H), 8.54 (s, 1H), 8.23 (d, J=8.62 Hz, 1H), 8.05 (d, J=8.13 Hz, 1H), 7.96-7.86 (m, 2H), 7.63-7.59 (m, 2H), 7.47 (t, J=7.65 Hz, 1H), 4.43 (t, J=5.12 Hz, 2H), 2.85-2.82 (m, 2H), 2.72-2.69 (m, 4H).

The following compounds in Table 5 were prepared as described above for N-(6-methanesulfonyl-1,3-benzothiazol-2-yl)naphthalene-2-carboxamide with the appropriate amine and carboxylic acid.

TABLE 5
Core modifications

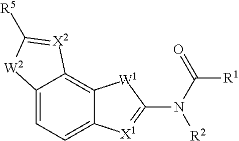

| | Acid | Amine | | | | | | | |
|---|---|---|---|---|---|---|---|---|---|
| Ex | (Int. | (Int. | | | | | | | |
| No. | No.) | No.) | Name | R1 | R5 | W¹ | X¹ | X² | W² |
| 48 | 5′ | 27 | N-{5-thia- 3,10,12- triazatricyclo[7.3. 0.0²,^{⁶}]dodeca- 1,3,6,8,11- pentaen-11- | 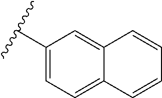 [SMILES: *c1ccc2ccccc2c1] | H | NH | N | N | S |
| | | | yl}naphthalene- | | | | | | |
| | | | 2-carboxamide | | | | | | |
| 49 | 10 | 36 | N-{3,10-dithia-5- azatricyclo[7.3.0. 0²,^{⁶}]dodeca- 1,4,6,8,11- pentaen-4-yl}-3- [4-(morpholin-4- | 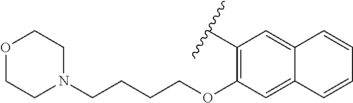 [SMILES: *c1cc2ccccc2cc1OCCCCN1CCOCC1] | H | S | N | CH | S |
| | | | yl)butoxy] | | | | | | |
| | | | naphthalene-2- | | | | | | |
| | | | carboxamide | | | | | | |
| 50 | 12 | 36 | 3-[4-(morpholin- 4-yl)butoxy]-N- {10-oxa-3-thia-5- azatricyclo[7.3.0. 0²,^{⁶}]dodeca- 1,4,6,8,11- | 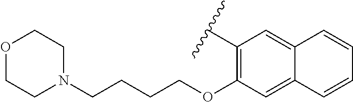 [SMILES: *c1cc2ccccc2cc1OCCCCN1CCOCC1] | H | S | N | CH | O |
| | | | pentaen-4- | | | | | | |
| | | | yl}naphthalene- | | | | | | |
| | | | 2-carboxamide | | | | | | |
| 51 | 12 | 35 | 3-[2-(morpholin- 4-yl)ethoxy]-N- {10-oxa-3-thia-5- azatricyclo [7.3.0.0²,^{⁶}] dodeca- | 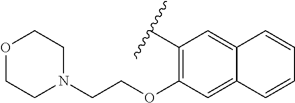 [SMILES: *c1cc2ccccc2cc1OCCN1CCOCC1] | H | S | N | CH | O |
| | | | 1,4,6,8,11- | | | | | | |
| | | | pentaen-4- | | | | | | |
| | | | yl}naphthalene- | | | | | | |
| | | | 2-carboxamide | | | | | | |
| 52 | 12 | 35 | 3-[2-(morpholin- 4-yl)ethoxy]-N- {10-oxa-3-thia- 5,12- diazatricyclo[7.3. 0.0²,^{⁶}]dodeca- | 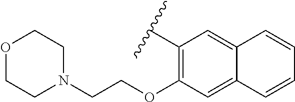 [SMILES: *c1cc2ccccc2cc1OCCN1CCOCC1] | H | S | N | N | O |
| | | | 1,4,6,8,11- | | | | | | |
| | | | pentaen-4- | | | | | | |
| | | | yl}naphthalene- | | | | | | |
| | | | 2-carboxamide | | | | | | |
| 53 | 12 | 36 | 3-[4-(morpholin- 4-yl)butoxy]-N- {10-oxa-3-thia- 5,12- diazatricyclo [7.3.0.0²,^{⁶}] | 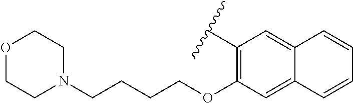 [SMILES: *c1cc2ccccc2cc1OCCCCN1CCOCC1] | H | S | N | N | O |
| | | | dodeca- | | | | | | |
| | | | 1,4,6,8,11- | | | | | | |
| | | | pentaen-4- | | | | | | |
| | | | yl}naphthalene- | | | | | | |
| | | | 2-carboxamide | | | | | | |
| 54 | 8 | 36 | N-{5,12-dithia-3- azatricyclo[7.3.0. 0²,^{⁶}]dodeca- 1,3,6,8,10- pentaen-11-yl}- 3-[4-(morpholin- | 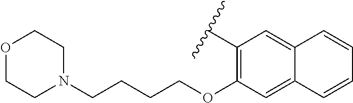 [SMILES: *c1cc2ccccc2cc1OCCCCN1CCOCC1] | H | S | CH | N | S |
| | | | 4- | | | | | | |
| | | | yl)butoxy] | | | | | | |
| | | | naphthalene-2- | | | | | | |
| | | | carboxamide | | | | | | |
| 55 | 8 | 35 | N-{5,12-dithia-3- azatricyclo[7.3.0. 0²,^{⁶}]dodeca- 1,3,6,8,10- pentaen-11-yl}- 3-[2-(morpholin- | 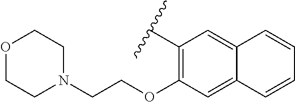 [SMILES: *c1cc2ccccc2cc1OCCN1CCOCC1] | H | S | CH | N | S |
| | | | 4- | | | | | | |
| | | | yl)ethoxy] | | | | | | |
| | | | naphthalene-2- | | | | | | |
| | | | carboxamide | | | | | | |
| 56 | 7 | 36 | 3-[4-(morpholin- 4-yl)butoxy]-N- {12-oxa-5-thia-3- azatricyclo [7.3.0.0²,^{⁶}] dodeca- | 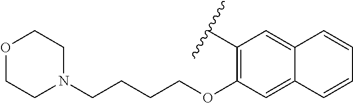 [SMILES: *c1cc2ccccc2cc1OCCCCN1CCOCC1] | H | O | CH | N | S |
| | | | 1,3,6,8,10- | | | | | | |
| | | | pentaen-11- | | | | | | |
| | | | yl}naphthalene- | | | | | | |
| | | | 2-carboxamide | | | | | | |
| 57 | 7 | 35 | 3-[2-(morpholin- 4-yl)ethoxy]-N- {12-oxa-5-thia-3- azatricyclo [7.3.0.0²,] dodeca- 1,3,6,8,10- | 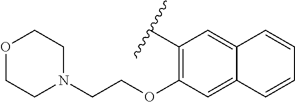 [SMILES: *c1cc2ccccc2cc1OCCN1CCOCC1] | H | O | C | N | S |
| | | | pentaen-11- | | | | | | |
| | | | yl}naphthalene- | | | | | | |
| | | | 2-carboxamide | | | | | | |
| 58 | 13 | 35 | 3-[2-(morpholin- 4-yl)ethoxy]-N- {3-oxa-10-thia- 5,12- diazatricyclo [7.3.0.0²,^{⁶}] | 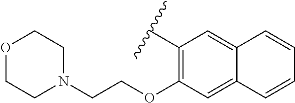 [SMILES: *c1cc2ccccc2cc1OCCN1CCOCC1] | H | O | N | N | S |
| | | | dodeca- | | | | | | |
| | | | 1,4,6,8,11- | | | | | | |
| | | | pentaen-4- | | | | | | |
| | | | yl}naphthalene- | | | | | | |
| | | | 2-carboxamide | | | | | | |
| 59 | 13 | 36 | 3-[4-(morpholin- 4-yl)butoxy]-N- {3-oxa-10-thia- 5,12- diazatricyclo [7.3.0.0²,] dodeca- | 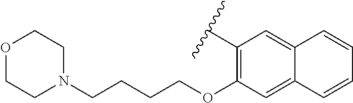 [SMILES: *c1cc2ccccc2cc1OCCCCN1CCOCC1] | H | O | N | N | S |
| | | | 1,4,6,8,11- | | | | | | |
| | | | pentaen-4- | | | | | | |
| | | | yl}naphthalene- | | | | | | |
| | | | 2-carboxamide | | | | | | |
| 60 | 8 | 16 | N-{5,12-dithia-3- azatricyclo[7.3.0. 0²,^{⁶}]dodeca- 1,3,6,8,10- pentaen-11-yl}- 1-methyl-1H- indole-2- | 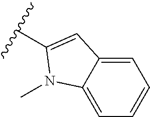 [SMILES: *c1cc2ccccc2n1C] | H | S | CH | N | S |
| | | | carboxamide | | | | | | |
| 61 | 8 | 46 | N-{5,12-dithia-3- azatricyclo[7.3.0. 0²,^{⁶}]dodeca- 1,3,6,8,10- pentaen-11-yl}- 3-[2-(morpholin- 4-yl)ethoxy]- [1,1′-biphenyl]- 4-carboxamide | 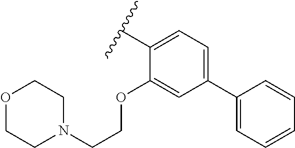 [SMILES: *c1ccc(-c2ccccc2)cc1OCCN1CCOCC1] | H | S | CH | N | S |
| 62 | 8 | 47 | N-{5,12-dithia-3- azatricyclo[7.3.0. 0²,^{⁶}]dodeca- 1,3,6,8,10- pentaen-11-yl}- 3-[4-(morpholin- 4-yl)butoxy]- [1,1′-biphenyl]- 4-carboxamide | 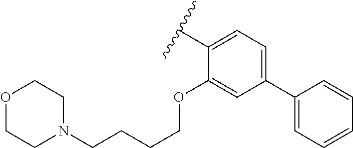 [SMILES: *c1ccc(-c2ccccc2)cc1OCCCCN1CCOCC1] | H | S | CH | N | S |
| 63 | 8 | 48 | N-{5,12-dithia-3- azatricyclo[7.3.0. 0²,^{⁶}]dodeca- 1,3,6,8,10- pentaen-11-yl}- 6-[2-(morpholin- 4-yl)ethoxy]-2H- 1,3- benzodioxole-5- | 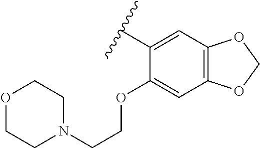 [SMILES: *c1cc2c(cc1OCCN1CCOCC1)OCO2] | H | S | CH | N | S |
| | | | carboxamide | | | | | | |
| 64 | 8 | 49 | N-{5,12-dithia-3- azatricyclo[7.3.0. 0²,^{⁶}]dodeca- 1,3,6,8,10- pentaen-11-yl}- 6-[2-(morpholin- 4-yl)ethoxy]-2H- 1,3- benzodioxole-5- | 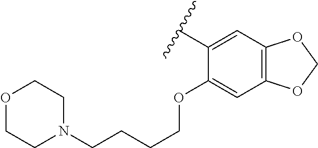 [SMILES: *c1cc2c(cc1OCCCCN1CCOCC1)OCO2] | H | S | CH | N | S |
| | | | carboxamide | | | | | | |
| 65 | 8 | 50 | N-{5,12-dithia-3- azatricyclo[7.3.0. 0²,^{⁶}]dodeca- 1,3,6,8,10- pentaen-11-yl}- 6-[2-(morpholin- 4-yl)ethoxy]-1- benzothiophene- 5-carboxamide | 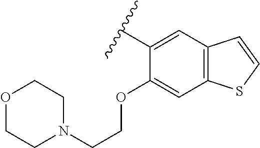 [SMILES: *c1cc2ccsc2cc1OCCN1CCOCC1] | H | S | CH | N | S |
| 66 | 8 | 51 | N-{5,12-dithia-3- azatricyclo[7.3.0. 0²,^{⁶}]dodeca- 1,3,6,8,10- pentaen-11-yl}- 6-[4-(morpholin- 4-yl)butoxy]-1- benzothiophene- 5-carboxamide | 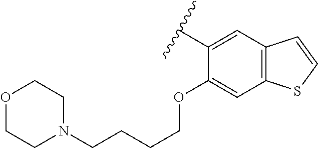 [SMILES: *c1cc2ccsc2cc1OCCCCN1CCOCC1] | H | S | CH | N | S |
| 67 | 8′ | 50 | N-{4-methoxy- 5,12-dithia-3- azatricyclo[7.3.0. 0²,^{⁶}]dodeca- 1,3,6,8,10- pentaen-11-yl}- 6-[2-(morpholin- 4-yl)ethoxy]-1- benzothiophene- | 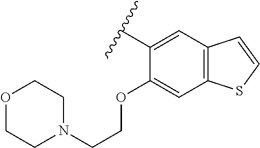 [SMILES: *c1cc2ccsc2cc1OCCN1CCOCC1] | OCH₃ | S | CH | N | S |
| | | | 5-carboxamide | | | | | | |
| 68 | 8′ | 51 | N-{4-methoxy- 5,12-dithia-3- azatricyclo[7.3.0. 0²,^{⁶}]dodcca- 1,3,6,8,10- pentaen-11-yl}- 6-[4-(morpholin- 4-yl)butoxy]-1- benzothiophene- | 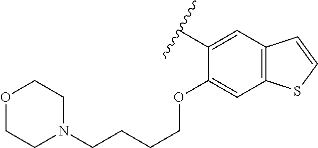 [SMILES: *c1cc2ccsc2cc1OCCCCN1CCOCC1] | OCH₃ | S | CH | N | S |
| | | | 5-carboxamide | | | | | | |
| 69 | 8 | 52 | N-{3,10-dithia- 5,12- diazatricyclo[7.3. 0.0²,]dodeca- 1,4,6,8,11- pentaen-4-yl}-1- methyl-5-[2- (morpholin-4- yl)ethoxy]-1H- | 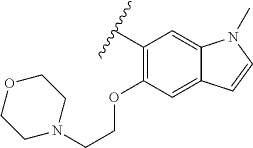 [SMILES: *c1cc2c(ccn2C)cc1OCCN1CCOCC1] | H | S | CH | N | S |
| | | | indole-6- | | | | | | |
| | | | carboxamide | | | | | | |
| 70 | No | 50 | 6-[2-(morpholin- 4-yl)ethoxy]-N- {4-oxo-5,12- dithia-3- azatricyclo[7.3.0. 0²,^{⁶}]dodeca- 1,6,8,10-tetraen- 11-yl}-1- benzothiophene- | 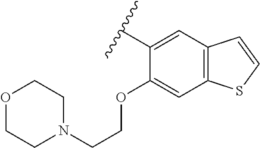 [SMILES: *c1cc2ccsc2cc1OCCN1CCOCC1] | OH | S | CH | N | S |
| | | | 5-carboxamide | | | | | | |
| 71 | 8 | 16 | N-{5,12-dithia-3- azatricyclo[7.3.0. 0²,]dodeca- 1,3,6,8,10- pentaen-11-yl}- 1-methyl-1H- | 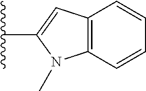 [SMILES: *c1cc2ccccc2n1C] | H | S | CH | N | S |
| | | | indole-2- | | | | | | |
| | | | carboxamide | | | | | | |
| 72 | 5 | 35 | 3-[2-(morpholin- 4-yl)ethoxy]-N- {3-thia-5,10,12- triazatricyclo[7.3. 0.0²,^{⁶}]dodeca- 1,4,6,8,11- | 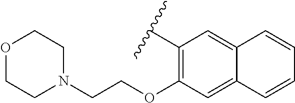 [SMILES: *c1cc2ccccc2cc1OCCN1CCOCC1] | H | S | N | N | NH |
| | | | pentaen-4- | | | | | | |
| | | | yl}naphthalene- | | | | | | |
| | | | 2-carboxamide | | | | | | |

NMR and LC/MS mass spectrometry data for the benzothiazolyl compounds of Table 5 are provided in Table 6 below.

TABLE 6
NMR and LC/MS Data

| | | LC/MS |
|---|---|---|
| Ex. No. | ¹H NMR | (MH⁺) |
| 48 | ¹H NMR (400 MHz, CDCl₃): δ 9.21 (s, 1H), 8.35 (dt, J = 1.69, | 345.4 |
| | 0.81 Hz, 1H), 8.07 (dt, J = 7.32, 1.78 Hz, 1H), 7.96-7.90 (m, | |
| | 1H), 7.89 (t, J = 1.02 Hz, 2H), 7.82 (d, J = 7.51 Hz, 1H), | |
| | 7.67-7.57 (m, 2H), 7.49 (d, J = 7.32 Hz, 1H) | |
| 49 | ¹H NMR (400 MHz, CDCl₃): δ 11.43 (s, 1H), 8.89 (s, 1H), | 518.6 |
| | 7.96 (d, J = 8.0 Hz, 1H), 7.91 (d, J = 8.4 Hz, 1H), 7.77 (d, J = 8.4 Hz, | |
| | 1H), 7.70 (d, J = 8.4 Hz, 1H), 7.64 (d, J = 5.2 Hz, 1H), 7.60 (t, J = 7.4 Hz, | |
| | 1H), 7.50 (d, J = 5.2 Hz, 1H), 7.47 (t, J = 7.4 Hz, 1H), | |
| | 4.45-4.38 (m, 2H), 4.30-4.10 (m, 2H), 4.00-3.80 (m, 2H), | |
| | 3.34-3.24 (m, 2H), 3.07-2.90 (m, 2H), 2.50-2.37 (m, 2H), 2.27-2.18 (m, | |
| | 2H), 1.74-1.59 (m, 2H) | |
| 50 | ¹H NMR (400 MHz, DMSO-d6): δ12.46 (s, 1H), 10.66-10.50 (br, | 502.6 |
| | 1H), 8.35 (s, 1H), 8.17 (d, J = 1.6 Hz, 1H), 8.01 (d, J = 8.4 Hz, | |
| | 1H), 7.90 (d, J = 8.4 Hz, 1H), 7.78-7.71 (m, 2H), 7.60 (t, J = 7.4 Hz, | |
| | 1H), 7.56 (s, 1H), 7.45 (t, J = 7.6 Hz, 1H), 7.33 (d, J = 1.6 Hz, | |
| | 1H), 4.30-4.24 (m, 2H), 3.95-3.55 (m, 4H), 3.29-2.90 (m, 4H), | |
| | 2.45-2.05 (m, 2H), 1.98-1.80 (m, 4H) | |
| 51 | ¹H NMR (400 MHz, DMSO-d6): δ12.66 (br, 1H), 11.57 (br, 1H), | 474.5 |
| | 8.29 (s, 1H), 8.17 (d, J = 2.0 Hz, 1H), 8.01 (d, J = 8.0 Hz, 1H), | |
| | 7.94 (d, J = 8.0 Hz, 1H), 7.77-7.71 (m, 2H), 7.64-7.59 (m, 2H), | |
| | 7.48 (d, J = 7.4 Hz, 1H), 7.34 (d, J = 2.0 Hz, 1H), 4.71-4.65 (m, | |
| | 2H), 3.95-3.82 (m, 4H), 3.66-3.59 (m, 4H), 3.21-3.09 (m, 2H) | |
| 52 | ¹H NMR (400 MHz, DMSO-d6) δ8.95 (s, 1H), 8.58 (s, 1H), | 475.5 |
| | 8.06 (d, J = 8.0 Hz, 1H), 7.93 (d, J = 4.4 Hz, 1H), 7.91 (d, J = 4.0 Hz, | |
| | 1H), 7.85 (d, J = 5.2 Hz, 1H), 7.65 (d, J = 4.0 Hz, 1H), 7.62 (d, J = 7.6 Hz, | |
| | 1H), 7.47 (t, J = 8.0 Hz, 1H), 4.47-4.42 (m, 2H), | |
| | 3.62-3.57 (m, 4H), 2.89-2.84 (m, 2H), 2.59-2.53 (m, 4H) | |
| 53 | ¹H NMR (400 MHz, CDCl₃): δ 11.57 (s, 1H), 8.94 (s, 1H), | 503.5 |
| | 8.24 (s, 1H), 7.96 (d, J = 8.0 Hz, 1H), 7.84 (d, J = 8.8 Hz, 1H), 7.77 (d, | |
| | J = 8.4 Hz, 1H), 7.68 (d, J = 8.8 Hz, 1H), 7.59 (t, J = 7.4 Hz, 1H), | |
| | 7.45 (t, J = 7.4 Hz, 1H), 7.32 (s, 1H), 4.43 (t, J = 6.2 Hz, 2H), | |
| | 3.74-3.65 (m, 4H), 2.58 (t, J = 7.4 Hz, 2H), 2.55-2.48 (m, 4H), | |
| | 2.24-2.14 (m, 2H), 1.98-1.88 (m, 2H) | |
| 54 | ¹H NMR (400 MHz, CDCl₃): δ 10.97 (s, 1H), 9.10 (s, 1H), | 518.6 |
| | 8.93 (s, 1H), 7.94 (d, J = 8.0 Hz, 1H), 7.85 (d, J = 8.4 Hz, 1H), 7.75 (d, | |
| | J = 8.4 Hz, 1H), 7.69 (d, J = 8.4 Hz, 1H), 7.56 (t, J = 7.4 Hz, 1H), | |
| | 7.44 (t, J = 7.4 Hz, 1H), 7.27 (s, 1H), 7.20 (s, 1H), 4.38 (t, J = 6.6 Hz, | |
| | 2H), 3.70 (t, J = 4.6 Hz, 4H), 2.54 (t, J = 7.4 Hz, 2H), | |
| | 2.51-2.45 (m, 4H), 2.20-2.12 (m, 2H), 1.92-1.83 (m, 2H) | |
| 55 | ¹H NMR (400 MHz, DMSO-d6): δ11.74 (s, 1H), 9.51 (s, 1H), | 490.6 |
| | 8.40 (s, 1H), 8.09 (d, J = 8.8 Hz, 1H), 8.02 (d, J = 7.6 Hz, 1H), | |
| | 7.90 (d, J = 8.0 Hz, 1H), 7.85 (d, J = 8.0 Hz, 1H), 7.63-7.56 (m, | |
| | 2H), 7.49-7.42 (m, 1H), 7.28 (br, 1H), 4.42-4.34 (m, 2H), | |
| | 3.53-3.44 (m, 4H), 2.89-2.82 (m, 2H), 2.58-2.53 (m, 4H) | |
| 56 | ¹H NMR (400 MHz, DMSO-d6): δ11.59 (s, 1H), 9.46 (s, 1H), | 502.6 |
| | 8.27 (s, 1H), 7.99 (t, J = 7.8 Hz, 2H), 7.88 (d, J = 8.4 Hz, 1H), | |
| | 7.74 (d, J = 8.0 Hz, 1H), 7.57 (t, J = 7.4 Hz, 1H), 7.52 (s, 1H), | |
| | 7.43 (t, J = 7.4 Hz, 1H), 7.06 (s, 1H), 4.25 (t, J = 5.6 Hz, 2H), | |
| | 3.39-3.30 (m, 4H), 2.36-2.26 (m, 2H), 2.25-2.10 (m, 4H), | |
| | 1.91-1.82 (m, 2H), 1.72-1.60 (m, 2H) | |
| 57 | ¹H NMR (400 MHz, CDCl₃): δ11.38 (s, 1H), 8.99 (s, 1H), 8.89 (s, | 474.5 |
| | 1H), 7.96 (d, J = 8.0 Hz, 1H), 7.77 (t, J = 9.2 Hz, 2H), 7.65 (d, J = 8.0 Hz, | |
| | 1H), 7.56 (t, J = 7.4 Hz, 1H), 7.44 (t, J = 7.6 Hz, 1H), | |
| | 7.29 (s, 1H), 7.20 (s, 1H), 4.52-4.42 (m, 2H), 3.75-3.65 (m, 4H), | |
| | 3.13-3.04 (m, 2H), 2.82-2.68 (m, 4H) | |
| 58 | ¹H NMR (400 MHz, DMSO-d6): δ 9.56 (s, 1H), 8.40 (s, 1H), | 475.5 |
| | 8.14 (d, J = 8.4 Hz, 1H), 8.03 (d, J = 8.0 Hz, 1H), 7.89 (d, J = 8.4 Hz, | |
| | 1H), 7.79 (d, J = 8.8 Hz, 1H), 7.62-7.57 (m, 2H), 7.48-7.43 (m, | |
| | 1H), 4.39-4.32 (m, 2H), 3.49-3.44 (m, 4H), 2.83-2.77 (m, 2H), | |
| | 2.49-2.44 (m, 4H) | |
| 59 | ¹H NMR (400 MHz, DMSO-d6): δ 12.16 (br, 1H), 9.57 (s, 1H), | 503.5 |
| | 8.26 (s, 1H), 8.16 (d, J = 8.4 Hz, 1H), 8.00 (d, J = 8.4 Hz, 1H), | |
| | 7.89 (d, J = 8.4 Hz, 1H), 7.79 (d, J = 8.4 Hz, 1H), 7.61-7.57 (m, | |
| | 1H), 7.52 (s, 1H), 7.47-7.43 (m, 1H), 4.21 (br, 2H), 3.91 (br, 2H), | |
| | 3.60-3.39 (m, 4H), 3.26-2.91 (m, 4H), 1.83 (br, 4H) | |
| 60 | 1H NMR (400 MHz, DMSO-d6) δ 11.53 (s, 1H), 8.34 (s, 1H), | 526.7 |
| | 7.91 (s, 1H), 7.81 (d, J = 8.4 Hz, 1H), 7.68 (d, J = 5.6 Hz, 1H), | |
| | 7.63 (d, J = 8.4 Hz, 1H), 7.51 (d, J = 5.6 Hz, 1H), 7.18 (s, 1H), | |
| | 4.36 (t, J = 5.6 Hz, 2H), 4.25 (s, 3H), 3.54-3.45 (m, 4H), 2.84 (t, J = 5.6 Hz, | |
| | 2H), 2.49-2.45 (m, 4H) | |
| 61 | ¹H NMR (400 MHz, DMSO-d6): δ 11.89 (s, 1H), 11.11 (br, 1H), | 516.6 |
| | 9.50 (s, 1H), 8.08 (d, J = 8.4 Hz, 1H), 7.85 (d, J = 8.4 Hz, 1H), | |
| | 7.81-7.80 (m, 2H), 7.75 (d, J = 8.0 Hz, 1H), 7.55-7.52 (m, 3H), | |
| | 7.48-7.44 (m, 2H), 7.35 (s, 1H), 4.71 (br, 2H), 3.90-3.81 (m, 4H), | |
| | 3.63-3.57 (m, 4H), 3.25-3.17 (m, 2H) | |
| 62 | ¹H NMR (400 MHz, CDCl₃): δ10.82 (s, 1H), 9.08 (s, 1H), 8.40 (d, | 544.7 |
| | J = 8.0 Hz, 1H), 7.85 (d, J = 8.4 Hz, 1H), 7.68 (d, J = 8.4 Hz, 1H), | |
| | 7.62 (d, J = 6.8 Hz, 2H), 7.48 (t, J = 7.2 Hz, 2H), 7.39-7.35 (m, | |
| | 1H), 7.37 (dd, J = 8.4, 1.2 Hz, 1H), 7.22-7.20 (s, 1H), 7.18 (s, | |
| | 1H), 4.36 (t, J = 6.8 Hz, 2H), 3.74-3.63 (m, 4H), 2.55-2.49 (m, | |
| | 2H), 2.50-2.40 (m, 4H), 2.17-2.08 (m, 2H), 1.89-1.80 (m, 2H) | |
| 63 | ¹H NMR (400 MHz, CDCl₃): δ11.34 (s, 1H), 9.49 (s, 1H), 8.07 (d, | 496.6 |
| | J = 8.4 Hz, 1H), 7.81 (d, J = 8.4 Hz, 1H), 7.41 (s, 1H), 7.26 (s, | |
| | 1H), 7.07 (s, 1H), 6.12 (s, 2H), 4.33 (t, J = 5.2 Hz, 2H), | |
| | 3.52-3.45 (m, 4H), 2.82 (t, J = 5.2 Hz, 2H), 2.49-2.46 (m, 4H) | |
| 64 | ¹H NMR (400 MHz, CDCl₃): δ 10.75 (s, 1H), 9.08 (s, 1H), | 524.6 |
| | 7.85 (d, J = 8.4 Hz, 1H), 7.77 (s, 1H), 7.67 (d, J = 8.4 Hz, 1H), 7.13 (s, | |
| | 1H), 6.60 (s, 1H), 6.03 (s, 2H), 4.22 (t, J = 6.4 Hz, 2H), | |
| | 3.71-3.65 (m, 4H), 2.52-2.40 (m, 6H), 2.10-2.02 (m, 2H), 1.83-1.75 (m, 2H) | |
| 65 | ¹H NMR (400 MHz, DMSO-d6): δ 11.58 (s, 1H), 9.50 (s, 1H), | 496.6 |
| | 8.35 (s, 1H), 8.08 (d, J = 8.4 Hz, 1H), 7.92 (s, 1H), 7.84 (d, J = 8.4 Hz, | |
| | 1H), 7.69 (d, J = 5.2 Hz, 1H), 7.51 (d, J = 5.6 Hz, 1H), | |
| | 7.29 (s, 1H), 4.37 (t, J = 5.2 Hz, 2H), 3.54-3.43 (m, 4H), 2.85 (t, J = 5.2 Hz, | |
| | 2H), 2.49 (br, 4H) | |
| 66 | ¹H NMR (400 MHz, CDCl₃): δ 10.89 (s, 1H), 9.09 (s, 1H), | 524.6 |
| | 8.81 (s, 1H), 7.85 (d, J = 8.4 Hz, 1H), 7.68 (d, J = 8.4 Hz, 1H), 7.48 (s, | |
| | 1H), 7.38-7.35 (m, 2H), 7.19 (s, 1H), 4.34 (t, J = 6.4 Hz, 2H), | |
| | 3.75-3.63 (m, 4H), 2.55-2.49 (m, 2H), 2.47 (br, 4H), 2.19-2.08 (m, | |
| | 2H), 1.89-1.79 (m, 2H) | |
| 67 | 1H NMR (400 MHz, DMSO-d6): δ 11.53 (s, 1H), 8.34 (s, 1H), | 526.7 |
| | 7.91 (s, 1H), 7.81 (d, J = 8.4 Hz, 1H), 7.68 (d, J = 5.6 Hz, 1H), | |
| | 7.63 (d, J = 8.4 Hz, 1H), 7.51 (d, J = 5.6 Hz, 1H), 7.18 (s, 1H), | |
| | 4.36 (t, J = 5.6 Hz, 2H), 4.25 (s, 3H), 3.54.3.45 (m, 4H), 2.84 (t, J = 5.6 Hz, | |
| | 2H), 2.49-2.45 (m, 4H) | |
| 68 | 1H NMR (400 MHz, DMSO-d6): δ 11.51 (s, 1H), 8.18 (s, 1H), | 554.7 |
| | 7.84 (s, 1H), 7.80 (d, J = 8.4 Hz, 1H), 7.66 (d, J = 5.6 Hz, 1H), | |
| | 7.62 (d, J = 8.4 Hz, 1H), 7.48 (d, J = 5.6 Hz, 1H), 7.15 (s, 1H), | |
| | 4.24 (s, 3H), 4.19 (t, J = 6.0 Hz, 2H), 3.44-3.35 (m, 4H), 2.27 (t, J = 7.2 Hz, | |
| | 2H), 2.25-2.13 (m, 4H), 1.87-1.77 (m, 2H), | |
| | 1.66-1.57 (m, 2H) | |
| 69 | 1H NMR (400 MHz, CDCl₃): δ 11.39 (s, 1H), 9.09 (s, 1H), | 493.6 |
| | 8.45 (s, 1H), 7.86 (d, J = 8.4 Hz, 1H), 7.68 (d, J = 8.4 Hz, 1H), 7.25 (s, | |
| | 1H), 7.21 (d, J = 2.8 Hz, 1H), 7.19 (s, 1H), 6.44 (dd, J = 3.2, 0.8 Hz, | |
| | 1H), 4.39 (t, J = 5.6 Hz, 2H), 3.87 (s, 3H), 3.75-3.70 (m, 4H), | |
| | 3.01 (t, J = 5.2 Hz, 2H), 2.67-2.60 (m, 4H) | |
| 70 | 1H NMR (400 MHz, DMSO-d6): δ 12.41 (s, 1H), 11.56 (s, 1H), | 512.6 |
| | 8.31 (s, 1H), 7.91 (s, 1H), 7.68 (d, J = 5.6 Hz, 1H), 7.51-7.49 (m, | |
| | 3H), 7.12 (s, 1H), 4.35 (t, J = 5.2 Hz, 2H), 3.53-3.44 (m, 4H), | |
| | 2.83 (t, J = 5.2 Hz, 2H), 2.49-2.45 (m, 4H) | |
| 71 | 1H NMR (400 MHz, DMSO-d6): δ 11.92 (s, 1H), 9.49 (s, 1H), | 364.5 |
| | 8.08 (d, J = 8.4 Hz, 1H), 7.87 (d, J = 8.4 Hz, 1H), 7.76 (d, J = 8.0 Hz, | |
| | 1H), 7.62 (d, J = 8.4 Hz, 1H), 7.48 (s, 1H), 7.38-7.34 (m, 2H), | |
| | 7.17 (t, J = 7.6 Hz, 1H), 4.10 (s, 3H) | |
| 72 | 1H NMR (400 MHz, DMSO-d6): δ 12.80-13.05 (m, 1H), | 474.6 |
| | 12.19-12.27 (m, 1H), 8.56-8.59 (m, 1H), 8.32-8.34 (m, 1H), 8.05 (d, J = 8.0 Hz, | |
| | 1H), 7.91 (d, J = 8.0 Hz, 1H), 7.60-7.76 (m, 4H), 7.47 (t, J = 7.6 Hz, | |
| | 1H), 4.45 (t, J = 5.2 Hz, 2H), 3.60 (t, J = 4.4 Hz, 4H), | |
| | 2.87 (t, J = 4.4 Hz, 2H), 2.56 (br, 4H) | |

### Example A

### Induction of IRF3-Dependent Gene Expression in THP1-Lucia™ ISG Cells

The compounds were evaluated in the THP1-Lucia™ ISG (interferon stimulated genes) reporter assay to determine if the compounds activate the IRF3 signaling pathway. The THP1-Lucia™ cells (InvivoGen) express the secreted luciferase (Lucia) reporter gene under the control of an IRF-inducible promoter. The reporter cell line was developed from human monocytic leukemia THP-1 cells.

The promoter was comprised of five IFN-stimulated response elements (ISRE) fused to an ISG54 minimal promoter which is unresponsive to NF-kB or AP-1 pathways. The secretion of luciferase by the THP1-Lucia™ ISG reporter cell line in response to small molecule RIG-I agonist compounds indicated the activation of the IRF3 pathway, since IRF3-deficient THP1-Lucia™ ISG IRF3−/−cells do not induce the secretion of luciferase in response to compounds. The IRF3-deficient THP1-Lucia™ ISG IRF3−/−reporter cell line was generated by CRISPR technology from the parent THP1-Lucia™ ISG reporter cell line.

THP1-Lucia™ ISG cells and IRF3-deficient THP1-Lucia™ ISG IRF3−/−cells were differentiated with PMA (100 ng/ml) and stimulated with compounds at the indicated concentrations (5 to 20 μM), positive control, or not treated (background). Luciferase secretion was quantified using the QUANTI-Luc luciferase assay system (InvivoGen) 18 h after stimulation. Data are shown as fold increase luciferase activity over background in Table 7 and represent the IRF3-dependent ISG54 promoter activity by the THP1-Lucia™ ISG cells in response to compounds. None of the listed 72 compounds induced luciferase expression in the IRF3 deficient THP1-Lucia™ IRF3−/−cells (less than 0.5 fold above baseline was considered below the level of quantitation). The fold increase of compounds (10 μM, *20 μM, **5 μM) induced IRF3 dependent luciferase activity is indicated as follows: “≠” indicates less than 2.4 fold increase; “+” indicates a 2.4-4.9 fold increase; “++” indicates a 5-9.9 fold increase; “+++” indicates a 10-19 fold increase; “++++” indicates a 20-39 fold increase; “+++++” indicates greater than or equal to 40 fold increase.

TABLE 7
Compound induced fold increase of
IRF3-depedent luciferase activity

| Ex. No. | THP1 ISG | THP1 ISG IRF3−/− |
|---|---|---|
| 1 | + | − |
| 2 | ++ | − |
| 3 | ++* | − |
| 4 | +++ | − |
| 5 | ++ | − |
| 6 | +++ | − |
| 7 | + | − |
| 8 | +++* | − |
| 9 | + | − |
| 10 | + | − |
| 11 | + | − |
| 12 | ++ | − |
| 13 | ++* | − |
| 14 | +++ | − |
| 15 | +* | − |
| 16 | +++ | − |
| 17 | +++ | − |
| 18 | ++++ | − |
| 19 | +++ | − |
| 20 | +++ | − |
| 21 | +++ | − |
| 22 | ++++ | − |
| 23 | ++ | − |
| 24 | ++++ | − |
| 25 | + | − |
| 27 | ++* | − |
| 28 | + | − |
| 29 | +++ | − |
| 30 | +++ | − |
| 31 | ++ | − |
| 32 | +++ | − |
| 33 | +++* | − |
| 34 | + | − |
| 35 | ++ | − |
| 36 | ++ | − |
| 37 | +++ | − |
| 38 | ++ | − |
| 39 | ++ | − |
| 40 | ++++ | − |
| 41 | + | − |
| 42 | ≠ | − |
| 43 | +++ | − |
| 44 | ++** | − |
| 45 | +++ | − |
| 46 | +** | − |
| 47 | + | − |
| 48 | ++* | − |
| 49 | +++ | − |
| 50 | +++ | − |
| 51 | +++ | − |
| 52 | + | − |
| 53 | ++ | − |
| 54 | +++ | − |
| 55 | +++ | − |
| 56 | + | − |
| 57 | +++ | − |
| 58 | + | − |
| 59 | ++ | − |
| 60 | +* | − |
| 61 | ++++ | − |
| 62 | +++ | − |
| 63 | ≠ | − |
| 64 | ≠ | − |
| 65 | + | − |
| 66 | + | − |
| 67 | N/A | − |
| 68 | N/A | − |
| 69 | N/A | − |
| 70 | N/A | − |
| 71 | +* | − |
| 72 | ++ | − |

*= 20 μM,
**= 5 μM compound concentrations. All other compounds were evaluated at 10 μM.
“N/A” indicates that the compound was not evaluated.

### Example B

### Induction of RIG-I Dependent CXCL10 Secretion by Murine CT26 Colon Carcinoma Cells in Response to Compounds

The CT26 murine colon carcinoma cell line (ATCC) was used to evaluate the induction of CXCL10 secretion. CXCL10 is an important chemokine in tumor immune biology that recruits tumor-specific T cells to the tumor. To confirm that compound-mediated CXCL10 production was RIG-I specific, RIG-1 deficient CT2-RIG-I−/−cells were generated by Kineta Inc. using CRISPR technology.

CT26 cells were seeded at a density of 1×10⁴ cells per well on a 96-well tissue culture plate in 100 μL of cell culture and cells were incubated at 37° C. and 5% CO₂ for 24 hr. Next, CT26 cells were treated with compounds at the indicated concentrations. CXCL10 was quantified by ELISA from supernatants taken 24 h after compound stimulation by use of the CXCL10 Duo Set ELISA kit (Cat #DY466, R&D, Minneapolis, Minn., USA) according to the manufacturer's instructions.

CXCL10 secretion by CT26 cells in response to compounds (in an amount of 5 to 20 μM) of the present disclosure is shown in Table 8. The compound-induced CXCL10 production was RIG-I dependent, since none of the compounds mediated CXCL10 secretion in RIG-I deficient CT26 RIG-I−/−cells (about 0 pg/mL of CXCL10, or below the level of quantitation). The compounds (10 μM, *20 μM, **5 μM) are indicated in the table as follows: “≠” indicates less than 100 pg/mL; “+” indicates 100-199 pg/mL; “++” indicates 200-399 pg/mL; “+++” indicates 400-799 pg/mL; “++++” indicates 800 to 1599 pg/mL; “+++++” indicates greater than or equal to 1600 pg/ml.

TABLE 8
RIG-I dependent CXCL10 secretion by murine CT26
colon carcinoma cells in response to compounds

| Ex. | CT26 | CT26 RIG-I−/− |
|---|---|---|
| 1 | ++++ | − |
| 2 | ++++* | − |
| 3 | ++* | − |
| 4 | +++ | − |
| 5 | +* | − |
| 6 | ++ | − |
| 7 | ++++ | − |
| 8 | +* | − |
| 9 | +++ | − |
| 10 | +++ | − |
| 11 | ++ | − |
| 12 | ++++ | − |
| 13 | +++ | − |
| 14 | ++* | − |
| 15 | ++ | − |
| 16 | ++ | − |
| 17 | ++ | − |
| 18 | +* | − |
| 19 | +++* | − |
| 20 | +++ | − |
| 21 | + | − |
| 22 | ++ | − |
| 23 | +++++* | − |
| 24 | +++ | − |
| 25 | ++ | − |
| 27 | ++ | − |
| 28 | ++ | − |
| 29 | + | − |
| 30 | +++ | − |
| 31 | ++ | − |
| 32 | ++ | − |
| 33 | ++++ | − |
| 34 | +++** | − |
| 35 | ++* | − |
| 36 | +++ | − |
| 37 | ++++ | − |
| 38 | +++ | − |
| 39 | ++ | − |
| 40 | ≠ | − |
| 41 | + | − |
| 42 | +* | − |
| 43 | +++ | − |
| 44 | +++ | − |
| 45 | +++ | − |
| 46 | +++ | − |
| 47 | + | − |
| 48 | ++++* | − |
| 49 | ++++ | − |
| 50 | ++ | − |
| 51 | +++ | − |
| 52 | + | − |
| 53 | +++++ | − |
| 54 | ++++ | − |
| 55 | ≠ | − |
| 56 | +++ | − |
| 57 | ++* | − |
| 58 | + | − |
| 59 | +++++ | − |
| 60 | +* | − |
| 61 | ++** | − |
| 62 | ++++ | − |
| 63 | ≠ | − |
| 64 | ≠ | − |
| 65 | ≠ | |
| 66 | +++++ | − |
| 67 | N/A | − |
| 68 | N/A | − |
| 69 | N/A | − |
| 70 | N/A | − |
| 71 | + | − |
| 72 | +++ | − |

*= 20 μM,
**= 5 μM compound concentrations. All other compounds were evaluated at 10 μM.
“N/A” indicates that the compound was not evaluated.

### Example

### Compound-Induced Immunogenic Cell Death in Murine Colon Carcinoma Cells

To determine if the RIG-I agonist compounds induce immunogenic cell death in cancer cells, induction of apoptosis and the translocation of calreticulin (CRT) to the cell surface in murine CT26 colon carcinoma cells were evaluated. The translocation of CRT occurs as part of a specific RIG-I dependent danger-signaling system, and the presence of CRT on the cell membrane promotes tumor antigen uptake by the dendritic cells and leads to the induction of an antigen-specific T cell response

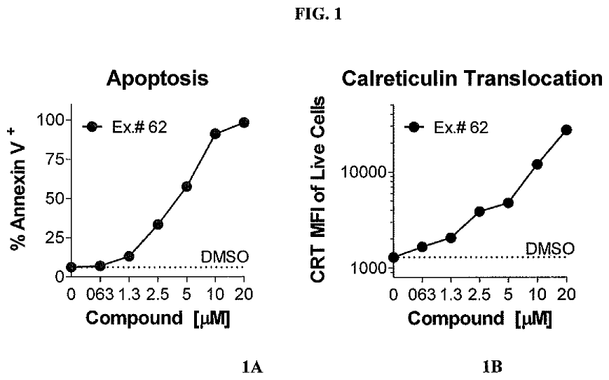

The induction of apoptosis and the CRT translocation were measured by flow cytometry. CT26 cells were seeded at a density of 4×10⁴ cells per well of a 6-well tissue culture plate in 2 mL of cell culture media and cells were incubated for 24 hr. Next, CT26 cells were treated with compounds at the indicated concentrations or treated with DMSO control (FIG. 1). Cells were harvested 18 h after treatment and then prepared for flow cytometry using an Annexin V staining kit (Biolegend) for quantification of apoptosis, an anti-CRT antibody (Abgent) for calreticulin translocation, and the Live/Dead-Violet staining kit (Thermofisher) for cell viability. Induction of apoptosis and translocation of calreticulin (CRT) to cell surface by live cells was determined by tri-color flow cytometry using FITC-labeled Annexin V, Live/Dead-iolet (LDV), and APC-anti-CRT. Apoptotic cells were defined as Annexin V⁺ and calreticulin translocation to cell surface was quantified by mean fluorescent intensity (MFI) of calreticulin⁺ live cells (CRT⁺ LDV⁻). A representative example of the induction of immunogenic cell death is shown in FIG. 1 for the compound of Example 62. The data represent typical dose titrations for induction apoptosis and calreticulin translocation by immunogenic cell death inducing compounds of this invention.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula I 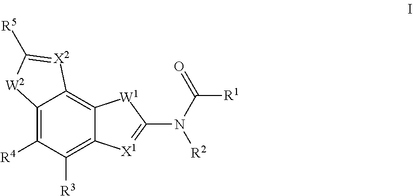
or a pharmaceutically acceptable salt thereof, wherein:
W¹ and W² are each independently selected from O, S, or NH;
X¹ and X² are each independently selected from N or CR^{X};
R^{X} is H or C₁₋₆ alkyl;
R¹ is a group having Formula (i), (ii), or (iii): 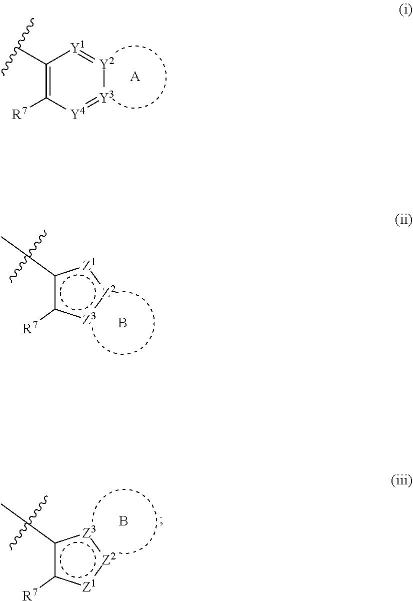
Y¹ is N or CR^{Y1};
Y² is N or CR^{Y2};
Y³ is N or CR^{Y3};
Y⁴ is N or CR^{Y4};
wherein not more than three of Y¹, Y², Y³, and Y⁴ are simultaneously N;
Z¹ is N, CR^{Z1}, O, S, or NR^{Z1};
Z² is N, CR^{Z2}, O, S, or NR^{Z2};
Z³ is N, CR^{Z3}, O, S, or NR^{Z3};
wherein the 5-membered ring containing Z¹, Z², and Z³ is aromatic;
Ring A is optionally present and represents a fused phenyl group, a fused 5-10 membered heteroaryl group, a fused C₅₋₇ cycloalkyl group, or a fused 5-10 membered heterocycloalkyl group, each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein the C₁₋₆ alkyl, C₂₋₆ alkenyl, and C₂₋₆ alkynyl is optionally substituted with 1, 2, or 3 substituents independently selected from Cy¹, Cy¹-C₁₋₄ alkyl, halo, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(S)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
wherein if Ring A is present, then Y² is CR^{Y2} and Y³ is CR^{Y3} wherein the R^{Y2} and R^{Y3} together with the carbon atoms to which they are attached form Ring A;
Ring B is optionally present and represents a fused phenyl group, a fused 5-10 membered heteroaryl group, a fused C₅₋₇ cycloalkyl group, or a fused 5-10 membered heterocycloalkyl group, each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein the C₁₋₆ alkyl, C₂₋₆ alkenyl, and C₂₋₆ alkynyl is optionally substituted with 1, 2, or 3 substituents independently selected from Cy¹, Cy¹-C₁₋₄ alkyl, halo, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
wherein if Ring B is present, then Z² is CR^{Z2} and Z³ is CR^{Z3} wherein the R^{Z2} and R^{Z3} together with the carbon atoms to which they are attached form Ring B;
R^{Y1}, R^{Y2}, R^{Y3}, R^{Y4}, R^{Z1}, R^{Z2}, and R^{Z3} are each independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{Y1}, R^{Y2}, R^{Y3}, R^{Y4}, R^{Z1}, R^{Z2}, and R^{Z3} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R² is H or C₁₋₄ alkyl;
R³ is H, halo, C₁₋₄ alkyl, C₁₋₄ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}C(S)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
R⁴ is H, halo, C₁₋₄ alkyl, C₁₋₄ haloalkyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}C(S)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};
R⁵ is H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}C(S)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R⁵ are each optionally substituted with 1, 2, 3, 4, or 5 substituents independently selected from Cy⁵, Cy⁵-C₁₋₄ alkyl, halo, C₁₋₆ alkyl, C₂₋₆ alkenyl, C₂₋₆ alkynyl, C₁₋₆ haloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}C(S)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
R⁷ is a group having the formula: —(C₁₋₂ alkyl)ₐ-(L¹)_{b}-(C₂₋₆ alkyl)_{c}-(L²)_{d}-Q;
L¹ is —O—, —S—, —NR⁸—, —CO—, —C(O)O—, —CONR⁸—, —SO—, —SO₂—, —SONR⁸—, —S(O)₂NR⁸—, or —NR⁸CONR⁹—;
L² is —O—, —S—, —NR¹⁰—, —CO—, —C(O)O—, —CONR¹⁰—, —SO—, —SO₂—, —SONR¹⁰—, —S(O)₂NR¹⁰—, or —NR¹⁰CONR¹¹—;
R⁸, R⁹, R¹⁰, and R¹¹ are each independently selected from H and C₁₋₄ alkyl;
a is 0 or 1;
b is 0 or 1;
c is 0 or 1;
d is 0 or 1;
wherein the sum of a and c is 1 or 2;
Q is H, halo, C₁₋₆ alkyl, C₂₋₆ alkenyl, C₂₋₆ alkynyl, C₁₋₆ haloalkyl, C₆₋₁₀ aryl, 5-10 membered heteroaryl, C₃₋₁₀ cycloalkyl, 5-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{a}, NR^{c}C(O)NR^{c}R^{d}, NR^{c}C(S)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}; wherein the C₁₋₆ alkyl, C₂₋₆ alkenyl, C₂₋₆ alkynyl, C₆₋₁₀ aryl, 5-10 membered heteroaryl, C₃₋₁₀ cycloalkyl, 5-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of Q are each optionally substituted by 1, 2, 3, 4 or 5 substituents selected from halo, C₁₋₆ alkyl, C₂₋₆ alkenyl, C₂₋₆ alkynyl, C₁₋₆ haloalkyl, phenyl, C₃₋₇ cycloalkyl, 5-10 membered heteroaryl, 4-10 membered heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{a}, NR^{c}C(O)NR^{c}R^{d}, NR^{c}C(S)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy⁵ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}C(S)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
each R^{a}, R^{b}, R^{c}, R^{d}, R^{a1}, R^{b1}, R^{c1}, R^{d1}, R^{a5}, R^{b5}, R^{c5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a5}, R^{b5}, R^{c5}, and R^{d5} is optionally substituted with 1, 2, 3, 4, or 5 substituents independently selected from Cy⁶, Cy⁶-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
each R^{a3}, R^{b3}, R^{c3}, R^{d3}, R^{a4}, R^{b4}, R^{c4}, and R^{d4} is independently selected from H and C₁₋₆ alkyl;
or R^{c} and R^{d} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
or R^{c1} and R^{d1} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
or R^{c5} and R^{d5} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
each Cy⁶ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
each R^{a6}, R^{b6}, R^{c6}, and R^{d6} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆haloalkoxy;
or R^{c6} and R^{d6} together with the N atom to which they are attached form a 3-7 membered heterocycloalkyl group optionally substituted with 1, 2, or 3 substituents independently selected from OH, CN, amino, halo, C₁₋₆ alkyl, C₁₋₆ alkoxy, C₁₋₆haloalkyl, and C₁₋₆haloalkoxy; and
each R^{e}, R^{e1}, R^{e3}, R^{e4}, R^{e5}, and R^{e6} is independently selected from H, C₁₋₄ alkyl, and CN,
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group;
wherein one or more ring-forming S atoms of any aforementioned heterocycloalkyl group is optionally substituted by one or two oxo (═O) groups;
wherein the compound is other than: N-{3,10-dithia-5,12-diazatricyclo[7.3.0.0²,⁶]dodeca-1,4,6,8,11-pentaen-4-yl}-3-[2-(morpholin-4-yl)ethoxy]naphthalene-2-carboxamide, or a pharmaceutically acceptable salt thereof.

2. The compound of claim 1, or a pharmaceutically acceptable salt thereof, wherein:
W¹ and W² are each independently selected from O, S, or NH;
X¹ and X² are each independently selected from N or CR^{X};
R^{X} is H or C₁₋₆ alkyl;
R¹ is a group having Formula (i): 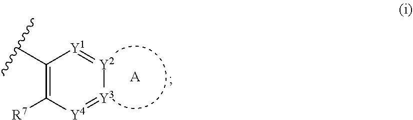
Y¹ is N or CR^{Y1};
Y² is N or CR^{Y2};
Y³ is N or CR^{Y3};
Y⁴ is N or CR^{Y4};
wherein not more than three of Y¹, Y², Y³, and Y⁴ are simultaneously N;
Ring A is a fused phenyl group, a fused 5-10 membered heteroaryl group, a fused C₅₋₇ cycloalkyl group, or a fused 5-10 membered heterocycloalkyl group, each optionally substituted with 1, 2, 3, 4, or 5 substituents independently selected from halo, C₁₋₆ alkyl, C₁₋₆ haloalkyl, CN, NO₂, OR^{a1}, SR^{a1}, C(O)R^{b1}, and NR^{c1}R^{d1};
wherein if Ring A is present, then Y² is CR^{Y2} and Y³ is CR^{Y3} wherein the R^{Y2} and R^{Y3} together with the carbon atoms to which they are attached form Ring A;
R^{Y1}, R^{Y2}, R^{Y3}, and R^{Y4} are each independently selected from H, halo, C₁₋₆ alkyl, C₁₋₆ haloalkyl, C₆₋₁₀ aryl, C₃₋₇ cycloalkyl, 5-10 membered heteroaryl, 4-10 membered heterocycloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, S(O)R^{b1}, S(O)NR^{c1}R^{d1}, S(O)₂R^{b1}, and S(O)₂NR^{c1}R^{d1}, wherein said C₁₋₆ alkyl, C₁₋₆ haloalkyl, C₆₋₁₀ aryl, C₃₋₇ cycloalkyl, 5-10 membered heteroaryl, and 4-10 membered heterocycloalkyl are each optionally substituted with 1, 2, 3, 4, or 5 substituents independently selected from halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, and OC(O)R^{b1};
R² is H;
R³ is H, halo, C₁₋₄ alkyl, C₁₋₄ haloalkyl, CN, NO₂, OR^{a3}, SR^{a3}, C(O)R^{b3}, C(O)NR^{c3}R^{d3}, C(O)OR^{a3}, NR^{c3}R^{d3}, S(O)NR^{c3}R^{d3}, S(O)₂R^{b3}, or S(O)₂NR^{c3}R^{d3};
R⁴ is H, halo, C₁₋₄ alkyl, C₁₋₄ haloalkyl, CN, NO₂, OR^{a4}, SR^{a4}, C(O)R^{b4}, C(O)NR^{c4}R^{d4}, C(O)OR^{a4}, NR^{c4}R^{d4}, S(O)NR^{c4}R^{d4}, S(O)₂R^{b4}, or S(O)₂NR^{c4}R^{d4};
R⁵ is R⁵ is H, halo, C₁₋₆ alkyl, C₁₋₆ haloalkyl, C₆₋₁₀ aryl, C₃₋₇ cycloalkyl, 5-10 membered heteroaryl, 4-10 membered heterocycloalkyl, CN, NO₂, OR^{a5}, SR^{a5}, C(O)R^{b5}, C(O)NR^{c5}R^{d5}, C(O)OR^{a5}, NR^{c5}R^{d5}, S(O)₂R^{b5}, and S(O)₂NR^{c5}R^{d5}; wherein said C₁₋₆ alkyl, C₁₋₆ haloalkyl, C₆₋₁₀ aryl, C₃₋₇ cycloalkyl, 5-10 membered heteroaryl, and 4-10 membered heterocycloalkyl of R⁵ are each optionally substituted with 1, 2, 3, 4, or 5 substituents independently selected from C₁₋₆ alkyl, CN, NO₂, OR^{a5}, SR^{a5}, C(O)R^{b5}, C(O)NR^{c5}R^{d5}, C(O)OR^{a5}, NR^{c5}R^{d5}, S(O)₂R^{b5}, and S(O)₂NR^{c5}R^{d5};
R⁷ is a group having the formula: L¹-(C₂₋₆ alkyl)-Q;
L¹ is —O—, —S—, —NR⁸—, —CO—, —C(O)O—, —CONR⁸—, or —NR⁸CONR⁹—;
Q is H, halo, C₁₋₆ alkyl, C₁₋₆ haloalkyl, C₆₋₁₀ aryl, 5-10 membered heteroaryl, C₃₋₁₀ cycloalkyl, 5-14 membered heterocycloalkyl, CN, NO₂, OR^{a}, SR^{a}, C(O)R^{b}, C(O)NR^{c}R^{d}, C(O)OR^{a}, OC(O)R^{b}, NR^{c}R^{d}, S(O)₂R^{b}, and S(O)₂NR^{c}R^{d}; wherein the C₁₋₆ alkyl, C₁₋₆ haloalkyl, C₆₋₁₀ aryl, 5-10 membered heteroaryl, C₃₋₁₀ cycloalkyl, and 5-14 membered heterocycloalkyl of Q are each optionally substituted by 1, 2, 3, 4 or 5 substituents selected from halo, C₁₋₆ alkyl, C₁₋₆ haloalkyl, phenyl, C₃₋₇ cycloalkyl, 5-10 membered heteroaryl, 4-10 membered heterocycloalkyl, CN, NO₂, OR^{a}, SR^{a}, C(O)R^{b}, NR^{c}R^{d}, S(O)₂R^{b}, and S(O)₂NR^{c}R^{d};
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{a}, R^{b}, R^{c}, R^{d}, R^{a1}, R^{b1}, R^{c1}, R^{d1}, R^{a5}, R^{b5}, R^{c5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a5}, R^{b5}, R^{c5}, and R^{d5} is optionally substituted with 1, 2, 3, 4, or 5 substituents independently selected from Cy⁶, Cy⁶-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
each R^{a3}, R^{b3}, R^{c3}, R^{d3}, R^{a4}, R^{b4}, R^{c4}, and R^{d4} is independently selected from H and C₁₋₆ alkyl;
or R^{c} and R^{d} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
or R^{c1} and R^{d1} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
or R^{c5} and R^{d5} together with the N atom to which they are attached form a 3-7 membered heterocycloalkyl group optionally substituted with 1, 2, or 3 substituents independently selected from halo, C₁₋₄ alkyl, C₁₋₄ haloalkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
each Cy⁶ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, NR^{c6}S(O)₂NR^{c6}R^{d6}, and S(O)₂NR^{c6}R^{d6};
each R^{a6}, R^{b6}, R^{c6}, and R^{d6} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆haloalkoxy;
or R^{c6} and R^{d6} together with the N atom to which they are attached form a 3-7 membered heterocycloalkyl group optionally substituted with 1, 2, or 3 substituents independently selected from OH, CN, amino, halo, C₁₋₆ alkyl, C₁₋₆alkoxy, C₁₋₆haloalkyl, and C₁₋₆haloalkoxy; and
each R^{e}, R^{e1}, R^{e3}, R^{e4}, R^{e5}, and R^{e6} is independently selected from H, C₁₋₄ alkyl, and CN,
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group;
wherein one or more ring-forming S atoms of any aforementioned heterocycloalkyl group is optionally substituted by one or two oxo (═O) groups;
wherein the compound is other than: N-{3,10-dithia-5,12-diazatricyclo[7.3.0.0²,⁶]dodeca-1,4,6,8,11-pentaen-4-yl}-3-[2-(morpholin-4-yl)ethoxy]naphthalene-2-carboxamide, or a pharmaceutically acceptable salt thereof.

3.-8. (canceled)

9. The compound of claim 1, or a pharmaceutically acceptable salt thereof, wherein W¹ and W² are each S.

10. The compound of claim 1, or a pharmaceutically acceptable salt thereof, wherein X¹ is N or CH.

11. (canceled)

12. The compound of claim 1, or a pharmaceutically acceptable salt thereof, wherein X² is N or CH.

13. (canceled)

14. The compound of claim 1, or a pharmaceutically acceptable salt thereof, wherein X¹ and X² are each N.

15. The compound of claim 1, or a pharmaceutically acceptable salt thereof, wherein R¹ is the group having Formula (i):

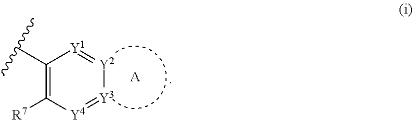

16. The compound of claim 1, or a pharmaceutically acceptable salt thereof, wherein Y¹ is CR^{Y1} or Y² is CR^{Y2} or Y³ is CR^{Y3} or Y⁴ is CR^{Y4}.

17.-23. (canceled)

24. The compound of claim 1, or a pharmaceutically acceptable salt thereof, wherein R^{Y2} is selected from H and C₆₋₁₀ aryl, wherein said C₆₋₁₀ aryl is optionally substituted with 1, 2, 3, 4, or 5 substituents independently selected from halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, and OC(O)R^{b1}.

25.-27. (canceled)

28. The compound of claim 1, or a pharmaceutically acceptable salt thereof, wherein R^{Y3} is selected from H and C₆₋₁₀ aryl, wherein said C₆₋₁₀ aryl is optionally substituted with 1, 2, 3, 4, or 5 substituents independently selected from halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, and OC(O)R^{b1}.

29.-34. (canceled)

35. The compound of claim 1, or a pharmaceutically acceptable salt thereof, wherein Y² is CR^{Y2} and Y³ is CR^{Y3}, and wherein the R^{Y2} and R^{Y3} together with the carbon atoms to which they are attached form Ring A.

36.-38. (canceled)

39. The compound of claim 1, or a pharmaceutically acceptable salt thereof, wherein A is a fused phenyl group, fused 1,3-dioxolanyl group, fused thiophenyl group, or fused pyrrolyl group.

40.-54. (canceled)

55. The compound of claim 1, or a pharmaceutically acceptable salt thereof, wherein R⁷ is a group having the formula: 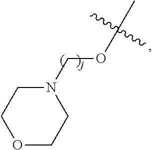
wherein j is 2, 3, 4, 5, or 6.

56. (canceled)

57. The compound of claim 1, or a pharmaceutically acceptable salt thereof, wherein L¹ is —O—, —S—, or —NR⁸—.

58. The compound of claim 1, or a pharmaceutically acceptable salt thereof, wherein L¹ is —O—.

59.-60. (canceled)

61. The compound of claim 1, or a pharmaceutically acceptable salt thereof, wherein Q is 5-14 membered heterocycloalkyl or NR^{c}R^{d}, wherein said 5-14 membered heterocycloalkyl is optionally substituted by 1, 2, 3, 4 or 5 substituents selected from halo, C₁₋₆ alkyl, C₁₋₆ haloalkyl, phenyl, C₃₋₇ cycloalkyl, 5-10 membered heteroaryl, 4-10 membered heterocycloalkyl, CN, NO₂, OR^{a}, SR^{a}, C(O)R^{b}, NR^{c}R^{d}, S(O)₂R^{b}, and S(O)₂NR^{c}R^{d}.

62. (canceled)

63. The compound of claim 1, or a pharmaceutically acceptable salt thereof, wherein Q is morpholinyl, piperidinyl, 2-oxa-6-azaspiro[3.3]heptanyl, 2-oxa-5-azabicyclo[2.2.1]heptanyl, or piperazinyl.

64.-65. (canceled)

66. The compound of claim 1, or a pharmaceutically acceptable salt thereof, wherein R^{d} is H or C₁₋₆ alkyl, wherein said C₁₋₆ alkyl is optionally substituted with OR^{a6}.

67. The compound of claim 1, or a pharmaceutically acceptable salt thereof, wherein R² is H.

68. (canceled)

69. The compound of claim 1, or a pharmaceutically acceptable salt thereof, wherein R³ is H, halo, or C₁₋₄ alkyl.

70.-75. (canceled)

76. The compound of claim 1, or a pharmaceutically acceptable salt thereof, wherein R⁵ is H, OR^{a5} or SR^{a5}.

77.-78. (canceled)

79. The compound of claim 1, having Formula IIa: 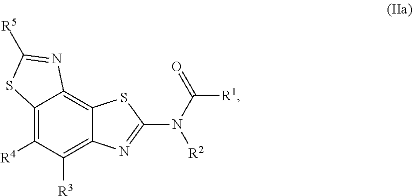
or a pharmaceutically acceptable salt thereof or 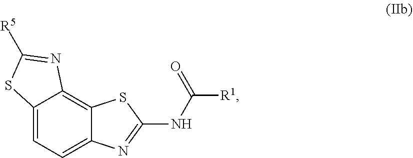
or a pharmaceutically acceptable salt thereof, or 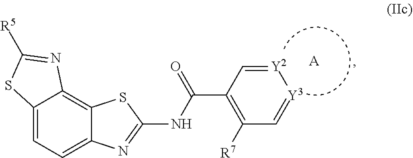
or a pharmaceutically acceptable salt thereof, or 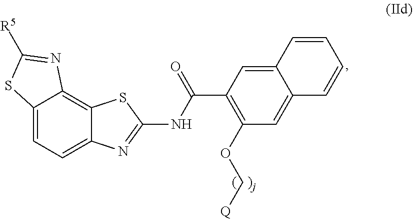
wherein j is 2, 3, 4, 5, or 6;
or a pharmaceutically acceptable salt thereof, or 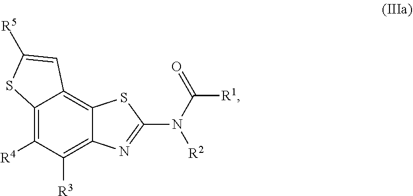
or a pharmaceutically acceptable salt thereof, or 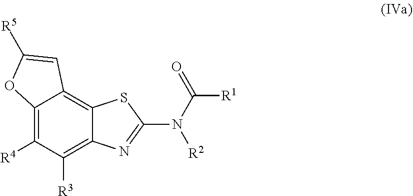
or a pharmaceutically acceptable salt thereof, or 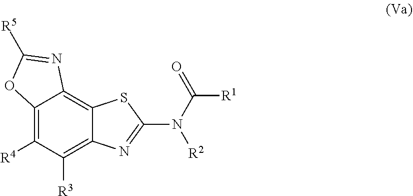
or a pharmaceutically acceptable salt thereof, or 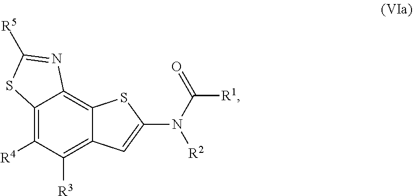
or a pharmaceutically acceptable salt thereof, or 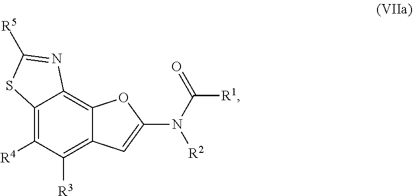
or a pharmaceutically acceptable salt thereof or 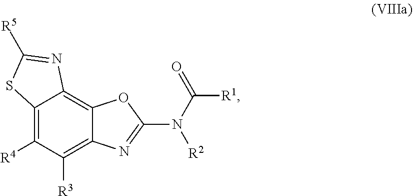
or a pharmaceutically acceptable salt thereof, 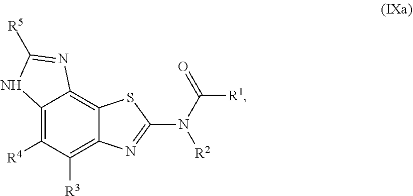
or a pharmaceutically acceptable salt thereof.

80.-89. (canceled)

90. The compound of claim 1, wherein the compound is selected from:
N-{3,10-dithia-5,12-diazatricyclo[7.3.0.0²,⁶]dodeca-1,4,6,8,11-pentaen-4-yl}-3-[2-(piperidin-1-yl)ethoxy]naphthalene-2-carboxamide;
N-{3,10-dithia-5,12-diazatricyclo[7.3.0.0²,⁶]dodeca-1,4,6,8,11-pentaen-4-yl}-3-[3-(morpholin-4-yl)propoxy]naphthalene-2-carboxamide;
N-{3,10-dithia-5,12-diazatricyclo[7.3.0.0²,⁶]dodeca-1,4,6,8,11-pentaen-4-yl}-3-[2-(oxan-4-yl)ethoxy]naphthalene-2-carboxamide;
N-{3,10-dithia-5,12-diazatricyclo[7.3.0.0²,⁶]dodeca-1,4,6,8,11-pentaen-4-yl}-3-[4-(morpholin-4-yl)butoxy]naphthalene-2-carboxamide;
N-{11-methoxy-3,10-dithia-5,12-diazatricyclo[7.3.0.0²,⁶]dodeca-1,4,6,8,11-pentaen-4-yl}-3-[2-(morpholin-4-yl)ethoxy]naphthalene-2-carboxamide;
N-{11-methoxy-3,10-dithia-5,12-diazatricyclo[7.3.0.0²,⁶]dodeca-1,4,6,8,11-pentaen-4-yl}-3-[2-(piperidin-1-yl)ethoxy]naphthalene-2-carboxamide;
N-{11-methoxy-3,10-dithia-5,12-diazatricyclo[7.3.0.0²,⁶]dodeca-1,4,6,8,11-pentaen-4-yl}-3-[3-(morpholin-4-yl)propoxy]naphthalene-2-carboxamide;
N-{11-methoxy-3,10-dithia-5,12-diazatricyclo[7.3.0.0²,⁶]dodeca-1,4,6,8,11-pentaen-4-yl}-3-[4-(morpholin-4-yl)butoxy]naphthalene-2-carboxamide;
N-{3,10-dithia-5,12-diazatricyclo[7.3.0.0²,⁶]dodeca-1,4,6,8,11-pentaen-4-yl}-3-(2-{2-oxa-5-azabicyclo[2.2.1]heptan-5-yl}ethoxy)naphthalene-2-carboxamide;
N-{3,10-dithia-5,12-diazatricyclo[7.3.0.0²^{⁶},]dodeca-1,4,6,8,11-pentaen-4-yl}-3-(2-{2-oxa-6-azaspiro[3.3]heptan-6-yl}ethoxy)naphthalene-2-carboxamide;
N-[11-(methylsulfanyl)-3,10-dithia-5,12-diazatricyclo[7.3.0.0²,⁶]dodeca-1,4,6,8,11-pentaen-4-yl]-3-[2-(morpholin-4-yl)ethoxy]naphthalene-2-carboxamide;
N-[11-(methylsulfanyl)-3,10-dithia-5,12-diazatricyclo[7.3.0.0²,⁶]dodeca-1,4,6,8,11-pentaen-4-yl]-3-[3-(morpholin-4-yl)propoxy]naphthalene-2-carboxamide;
N-[11-(methylsulfanyl)-3,10-dithia-5,12-diazatricyclo[7.3.0.0²,⁶]dodeca-1,4,6,8,11-pentaen-4-yl]-3-[4-(morpholin-4-yl)butoxy]naphthalene-2-carboxamide;
N-{3,10-dithia-5,12-diazatricyclo[7.3.0.0²,⁶]dodeca-1,4,6,8,11-pentaen-4-yl}-3-[4-(morpholin-4-yl)butoxy]-[1,1′-biphenyl]-4-carboxamide;
N-{3,10-dithia-5,12-diazatricyclo[7.3.0.0²,⁶]dodeca-1,4,6,8,11-pentaen-4-yl}-6-[4-(morpholin-4-yl)butoxy]naphthalene-2-carboxamide;
N-{3,10-dithia-5,12-diazatricyclo[7.3.0.0²,⁶]dodeca-1,4,6,8,11-pentaen-4-yl}-6-[2-(morpholin-4-yl)ethoxy]-2H-1,3-benzodioxole-5-carboxamide;
N-{3,10-dithia-5,12-diazatricyclo[7.3.0.0²,⁶]dodeca-1,4,6,8,11-pentaen-4-yl}-6-[4-(morpholin-4-yl)butoxy]-2H-1,3-benzodioxole-5-carboxamide;
N-{3,10-dithia-5,12-diazatricyclo[7.3.0.0²,⁶]dodeca-1,4,6,8,11-pentaen-4-yl}-6-[2-(morpholin-4-yl)ethoxy]-1-benzothiophene-5-carboxamide;
N-{3,10-dithia-5,12-diazatricyclo[7.3.0.0²,⁶]dodeca-1,4,6,8,11-pentaen-4-yl}-6-[4-(morpholin-4-yl)butoxy]-1-benzothiophene-5-carboxamide;
[N-(7-hydroxybenzo[1,2-d:3,4-d′]bis(thiazole)-2-yl)-3-(4-morpholinobutoxy)-2-naphthamide hydrochloride];
N-{3,10-Dithia-5,12-diazatricyclo[7.3.0.0^{2,6}]dodeca-1,4,6,8,11-pentaen-4-yl}-3-{2-[ethyl(2-hydroxyethyl)amino]ethoxy}naphthalene-2-carboxamide carboxamide;
N-{3,10-dithia-5,12-diazatricyclo[7.3.0.0²,⁶]dodeca-1,4,6,8,11-pentaen-4-yl}-3-{2-[(2-hydroxyethyl)amino]ethoxy}naphthalene-2-carboxamide;
N-{3,10-Dithia-5,12-diazatricyclo[7.3.0.0^{2,6}]dodeca-1,4,6,8,11-pentaen-4-yl}-3-[2-(piperazin-1-yl)ethoxy]naphthalene-2-carboxamide;
N-{3,10-dithia-5-azatricyclo[7.3.0.0²,⁶]dodeca-1,4,6,8,11-pentaen-4-yl}-3-[4-(morpholin-4-yl)butoxy]naphthalene-2-carboxamide;
3-[4-(morpholin-4-yl)butoxy]-N-{10-oxa-3-thia-5-azatricyclo[7.3.0.0²,⁶]dodeca-1,4,6,8,11-pentaen-4-yl}naphthalene-2-carboxamide;
3-[2-(morpholin-4-yl)ethoxy]-N-{10-oxa-3-thia-5-azatricyclo[7.3.0.0²,⁶]dodeca-1,4,6,8,11-pentaen-4-yl}naphthalene-2-carboxamide;
3-[2-(morpholin-4-yl)ethoxy]-N-{10-oxa-3-thia-5,12-diazatricyclo[7.3.0.0²,⁶]dodeca-1,4,6,8,11-pentaen-4-yl}naphthalene-2-carboxamide;
3-[4-(morpholin-4-yl)butoxy]-N-{10-oxa-3-thia-5,12-diazatricyclo [7.3.0.0²,⁶]dodeca-1,4,6,8,11-pentaen-4-yl}naphthalene-2-carboxamide;
N-{5,12-dithia-3-azatricyclo[7.3.0.0²,⁶]dodeca-1,3,6,8,10-pentaen-11-yl}-3-[4-(morpholin-4-yl)butoxy]naphthalene-2-carboxamide;
N-{5,12-dithia-3-azatricyclo[7.3.0.0²,⁶]dodeca-1,3,6,8,10-pentaen-11-yl}-3-[2-(morpholin-4-yl)ethoxy]naphthalene-2-carboxamide;
3-[4-(morpholin-4-yl)butoxy]-N-{12-oxa-5-thia-3-azatricyclo[7.3.0.0²,⁶]dodeca-1,3,6,8,10-pentaen-11-yl}naphthalene-2-carboxamide;
3-[2-(morpholin-4-yl)ethoxy]-N-{12-oxa-5-thia-3-azatricyclo[7.3.0.0²,⁶]dodeca-1,3,6,8,10-pentaen-11-yl}naphthalene-2-carboxamide;
3-[2-(morpholin-4-yl)ethoxy]-N-{3-oxa-10-thia-5,12-diazatricyclo [7.3.0.0²,⁶]dodeca-1,4,6,8,11-pentaen-4-yl}naphthalene-2-carboxamide;
3-[4-(morpholin-4-yl)butoxy]-N-{3-oxa-10-thia-5,12-diazatricyclo [7.3.0.0²,⁶]dodeca-1,4,6,8,11-pentaen-4-yl}naphthalene-2-carboxamide;
N-{5,12-dithia-3-azatricyclo[7.3.0.0²,⁶]dodeca-1,3,6,8,10-pentaen-11-yl}-3-[2-(morpholin-4-yl)ethoxy]-[1,1′-biphenyl]-4-carboxamide;
N-{5,12-dithia-3-azatricyclo[7.3.0.0²,⁶]dodeca-1,3,6,8,10-pentaen-11-yl}-3-[4-(morpholin-4-yl)butoxy]-[1,1′-biphenyl]-4-carboxamide;
N-{5,12-dithia-3-azatricyclo[7.3.0.0²,⁶]dodeca-1,3,6,8,10-pentaen-11-yl}-6-[2-(morpholin-4-yl)ethoxy]-2H-1,3-benzodioxole-5-carboxamide;
N-{5,12-dithia-3-azatricyclo[7.3.0.0²,⁶]dodeca-1,3,6,8,10-pentaen-11-yl}-6-[2-(morpholin-4-yl)ethoxy]-2H-1,3-benzodioxole-5-carboxamide;
N-{5,12-dithia-3-azatricyclo[7.3.0.0²,⁶]dodeca-1,3,6,8,10-pentaen-11-yl}-6-[2-(morpholin-4-yl)ethoxy]-1-benzothiophene-5-carboxamide;
N-{5,12-dithia-3-azatricyclo[7.3.0.0²,⁶]dodeca-1,3,6,8,10-pentaen-11-yl}-6-[4-(morpholin-4-yl)butoxy]-1-benzothiophene-5-carboxamide;
N-{4-methoxy-5,12-dithia-3-azatricyclo[7.3.0.0²,⁶]dodeca-1,3,6,8,10-pentaen-11-yl}-6-[2-(morpholin-4-yl)ethoxy]-1-benzothiophene-5-carboxamide;
N-{4-methoxy-5,12-dithia-3-azatricyclo[7.3.0.0²,⁶]dodeca-1,3,6,8,10-pentaen-11-yl}-6-[4-(morpholin-4-yl)butoxy]-1-benzothiophene-5-carboxamide;
N-{3,10-dithia-5,12-diazatricyclo[7.3.0.0²^{⁶},]dodeca-1,4,6,8,11-pentaen-4-yl}-1-methyl-5-[2-(morpholin-4-yl)ethoxy]-1H-indole-6-carboxamide;
6-[2-(morpholin-4-yl)ethoxy]-N-{4-oxo-5,12-dithia-3-azatricyclo[7.3.0.0²,⁶]dodeca-1,6,8,10-tetraen-11-yl}-1-benzothiophene-5-carboxamide; and
3-[2-(morpholin-4-yl)ethoxy]-N-{3-thia-5,10,12-triazatricyclo[7.3.0.0²,⁶]dodeca-1,4,6,8,11-pentaen-4-yl}naphthalene-2-carboxamide;
or a pharmaceutically acceptable salt thereof.

91. A compound selected from:
3,5-dimethoxy-N-{11-methyl-3,10-dithia-5,12-diazatricyclo[7.3.0.0²^{⁶},]dodeca-1,4,6,8,11-pentaen-4-yl}benzamide;
4-(diethyl sulfamoyl)-N-{11-methyl-3,10-dithia-5,12-diazatricyclo[7.3.0.0²,⁶]dodeca-1,4,6,8,11-pentaen-4-yl}benzamide;
N-{3,10-dithia-5,12-diazatricyclo[7.3.0.0²,⁶]dodeca-1,4,6,8,11-pentaen-4-yl}-2H-1,3-benzodioxole-5-carboxamide;
N-{11-methyl-3,10-dithia-5,12-diazatricyclo[7.3.0.0²,⁶]dodeca-1,4,6,8,11-pentaen-4-yl}-4-(pentyloxy)benzamide;
4-(dimethylamino)-N-{11-methyl-3,10-dithia-5,12-diazatricyclo[7.3.0.0²,⁶]dodeca-1,4,6,8,11-pentaen-4-yl}benzamide;
4-chloro-N-{3,10-dithia-5,12-diazatricyclo[7.3.0.0²,⁶]dodeca-1,4,6,8,11-pentaen-4-yl}-3-(trifluoromethyl)benzamide;
N-{3,10-dithia-5,12-diazatricyclo[7.3.0.0²,⁶]dodeca-1,4,6,8,11-pentaen-4-yl}-3-(trifluoromethyl)benzamide;
N-{3,10-dithia-5,12-diazatricyclo[7.3.0.0²,⁶]dodeca-1,4,6,8,11-pentaen-4-yl}-3-nitrobenzamide;
N-(3-bromophenyl)-11-methyl-3,10-dithia-5,12-diazatricyclo[7.3.0.0²,⁶]dodeca-1,4,6,8,11-pentaene-4-carboxamide;
N-{3,10-dithia-5,12-diazatricyclo[7.3.0.0²,⁶]dodeca-1,4,6,8,11-pentaen-4-yl}-1-benzothiophene-2-carboxamide;
N-{3,10-dithia-5,12-diazatricyclo[7.3.0.0²,⁶]dodeca-1,4,6,8,11-pentaen-4-yl}-2,1,3-benzothiadiazole-5-carboxamide;
N-{3,10-dithia-5,12-diazatricyclo[7.3.0.0²,⁶]dodeca-1,4,6,8,11-pentaen-4-yl}-5,6,7,8-tetrahydronaphthalene-2-carboxamide;
N-{3,10-dithia-5,12-diazatricyclo[7.3.0.0²,⁶]dodeca-1,4,6,8,11-pentaen-4-yl}-1-benzothiophene-5-carboxamide;
N-{3,10-dithia-5,12-diazatricyclo[7.3.0.0²^{⁶},]dodeca-1,4,6,8,11-pentaen-4-yl}-1-benzofuran-5-carboxamide;
N-{3,10-dithia-5,12-diazatricyclo[7.3.0.0²,⁶]dodeca-1,4,6,8,11-pentaen-4-yl}-3-methoxynaphthalene-2-carboxamide;
N-{3,10-dithia-5,12-diazatricyclo[7.3.0.0²^{⁶}]dodeca-1,4,6,8,11-pentaen-4-yl}-1-methyl-1H-indole-2-carboxamide;
N-{11-ethyl-3,10-dithia-5,12-diazatricyclo[7.3.0.0²,⁶]dodeca-1,4,6,8,11-pentaen-4-yl}naphthalene-2-carboxamide;
N-[11-(methylsulfanyl)-3,10-dithia-5,12-diazatricyclo[7.3.0.0²,⁶]dodeca-1,4,6,8,11-pentaen-4-yl]naphthalene-2-carboxamide;
N-{3,10-dithia-5,12-diazatricyclo[7.3.0.0²,⁶]dodeca-1,4,6,8,11-pentaen-4-yl}-1-methyl-1H-indole-6-carboxamide;
N-{3,10-dithia-5,12-diazatricyclo[7.3.0.0²,⁶]dodeca-1,4,6,8,11-pentaen-4-yl}-[1,1′-biphenyl]-4-carboxamide;
N-{11-methoxy-3,10-dithia-5,12-diazatricyclo[7.3.0.0²,⁶]dodeca-1,4,6,8,11-pentaen-4-yl}naphthalene-2-carboxamide;
N-{11-methyl-3,10-dithia-5,12-diazatricyclo[7.3.0.0²,⁶]dodeca-1,4,6,8,11-pentaen-4-yl}naphthalene-2-carboxamide;
N-{5-thia-3,10,12-triazatricyclo[7.3.0.0²,⁶]dodeca-1,3,6,8,11-pentaen-11-yl}naphthalene-2-carboxamide;
N-{5,12-dithia-3-azatricyclo[7.3.0.0²^{⁶},]dodeca-1,3,6,8,10-pentaen-11-yl}-1-methyl-1H-indole-2-carboxamide; and
N-{5,12-dithia-3-azatricyclo[7.3.0.0²,⁶]dodeca-1,3,6,8,10-pentaen-11-yl}-1-methyl-1H-indole-2-carboxamide;
or a pharmaceutically acceptable salt thereof.

92. A pharmaceutical composition comprising a compound of claim 1, or a pharmaceutically acceptable salt thereof, and at least one pharmaceutically acceptable carrier.

93.-98. (canceled)

99. A method for treating cancer in a subject, said method comprising administering to the subject a therapeutically effective amount of the compound of claim 1, or a pharmaceutically acceptable salt thereof, wherein the cancer is selected from breast cancer, carcinoid cancer, cervical cancer, colorectal cancer, endometrial cancer, glioma, head and neck cancer, liver cancer, lung cancer, lymphoma, melanoma, ovarian cancer, pancreatic cancer, prostate cancer, renal cancer, skin cancer, stomach cancer, testicular cancer, thyroid cancer, and urethelial cancer.

100.-102. (canceled)

103. A method for treating a cancer in a subject, said method comprising administering to the subject a therapeutically effective amount of the following compound:
N-{3,10-dithia-5,12-diazatricyclo[7.3.0.0²,⁶]dodeca-1,4,6,8,11-pentaen-4-yl}-3-[2-(morpholin-4-yl)ethoxy]naphthalene-2-carboxamide;
or a pharmaceutically acceptable salt thereof, wherein the cancer is selected from breast cancer, carcinoid cancer, cervical cancer, colorectal cancer, endometrial cancer, glioma, head and neck cancer, liver cancer, lung cancer, lymphoma, melanoma, ovarian cancer, pancreatic cancer, prostate cancer, renal cancer, skin cancer, stomach cancer, testicular cancer, thyroid cancer, and urethelial cancer.

104.-105. (canceled)

106. A pharmaceutical composition comprising a compound of claim 91, or a pharmaceutically acceptable salt thereof, and at least one pharmaceutically acceptable carrier.
